(19) United States
(12) Patent Application Publication
(10) Pub. No.: US 2021/0052580 A1
(43) Pub. Date: Feb. 25, 2021

(54) Combination Compositions and Therapies Comprising 4-Methyl-5-(Pyrazin-2-yl)-3H-1,2-Dithiole-3-Thione, and Methods of Making and Using Same

(71) Applicant: ST IP Holding AG, Zug (CH)

(72) Inventors: Bomi FRAMROZE, Menlo Park, CA (US); Jeffrey A. GELFAND, Cambridge, MA (US)

(21) Appl. No.: 17/013,142

(22) Filed: Sep. 4, 2020

Related U.S. Application Data
(63) Continuation of application No. PCT/IB2019/000235, filed on Mar. 6, 2019.

(30) Foreign Application Priority Data
Mar. 7, 2018 (IN) 201811008427

(51) Int. Cl. A61K 31/497; A61K 47/26; A61K 47/32; A61K 31/122; A61K 31/4375; A61K 31/155; A61P 9/10; A01N 1/02; A61K 31/12; A61K 31/05; A61K 31/26; A61K 31/194; A61K 31/015

(52) U.S. Cl. CPC A61K 31/497; A61K 47/26; A61K 47/32; A61K 31/122; A61K 31/4375; A61K 31/155; A61K 31/015; A01N 1/0226; A61K 31/12; A61K 31/05; A61K 31/26; A61K 31/194; A61P 9/10

(57) ABSTRACT

This disclosure provides, among other things, compositions comprising quantities of oltipraz, either in recrystallized or formulated crystal form, and a composition that reduces the rate of cellular oxygen consumption such as atovaquone, as well as methods of making such compositions, and methods of treating patients using such compositions. This disclosure also provides, among other things, compositions comprising quantities of oltipraz, either in recrystallized or formulated crystal form, for use in treating patients who may experience ischemia and/or reperfusion injury.

### CROSS-REFERENCE TO RELATED APPLICATIONS

This application claims the benefit of Indian Patent Application No: 201811008427, filed on 7 Mar. 2018, which is hereby incorporated by reference in its entirety.

### FIELD

The disclosure herein relates to new pharmaceutical formulations, compositions and therapies comprising the compound 4-methyl-5-(pyrazin-2-yl)-3H-1,2-dithiole-3-thione (also known as oltipraz), optionally in combination with at least one additional active pharmaceutical ingredient such as atovaquone that can reduce cellular oxygen consumption rate (OCR-API). Methods of making and using such formulations, compositions and combination therapies are also disclosed.

### BACKGROUND

Mucositis is the painful inflammation and ulceration of mucous membranes often caused by chemo-/radio-therapy for cancer. Mucositis typically occurs in the gastrointestinal (GI) tract. e.g. in the oral (e.g. buccal) cavity. Oral and gastrointestinal (GI) mucositis is a common, painful side-effect of patients undergoing treatments such as high-dose chemotherapy, hematopoietic stem cell transplantation and the like.

Lesions of mucositis are characterized by mucosal breakdown resulting in extensive, deep ulcerations. Among granulocytopenic cancer patients, the loss in mucosal integrity created by ulceration results in the generation of a portal of entry for indigenous oral bacteria that often leads to sepsis or bacteremia. Mucositis occurs to some degree in more than one third of patients receiving anti-neoplastic drug therapy. The frequency and severity are significantly greater among patients who are treated with induction therapy for leukemia or with many of the conditioning regimens for hematopoietic stem cell marrow transplant. Moderate to severe mucositis occurs in virtually all patients who receive radiation therapy for tumors of the head and neck and typically begins with cumulative exposures of 20 Gy and then worsens as total doses of 60 Gy or more are reached.

Clinically mucositis progresses through three stages:

1. Early, painful mucosal erythema, which can be palliated with local anesthetics or non-narcotic analgesics.

2. Painful ulceration with pseudomembrane formation. Pain is often of such intensity as to require parenteral narcotic analgesia.

3. Spontaneous healing, occurring about 2-4 weeks after cessation of anti-neoplastic therapy.

To date, therapy for mucositis is predominantly palliative and focused on pain control and maintenance of nutrition. For example, oral mucositis is in practice often addressed only by palliative measures such as improvements in oral hygiene, alone or in combination with analgesic therapy such as administration of lidocaine. Such approaches have typically low efficacy and are insufficient for addressing severe cases of mucositis. Even opioids are often insufficient to control mucositis pain. Various pharmaceutical therapies for mucositis have been proposed however to date there remains a clear need for improved treatments for mucositis. In this context, oltipraz (4-methyl-5-(pyrazin-2-yl)-3H-1,2-dithiole-3-thione) has been suggested as a potential candidate. See, e.g., Fahl et al. PCT/US2001/014464 (published as WO2001085142) and Prendergast PCT/EP2008/052969 (published as WO 2008/110585).

Oltipraz is known to exist in crystalline form. To date, known crystalline oltipraz formulations, which are prepared by recrystallizing oltipraz (see, e.g., WO2016207914), comprise a mixture of oltipraz crystals of varying sizes up to millimeters in length along the longest axis, which crystals are highly insoluble in water and have poor bioavailability when administered topically or orally. As discussed below, it has been found that administering certain formulations of oltipraz can provide a beneficial, protective effects to mucosal cells. Despite such beneficial benefits, however, damage to mucosal cells can still occur during the course of treatments such as chemotherapy and/or radiation therapy. Thus, there remains a need for new compositions and/or combinations of ingredients that can provide an enhanced protective effect as compared to administering oltipraz alone.

Some active pharmaceutical ingredients are capable of reducing cellular oxygen consumption rate (OCR-APIs). Sec, e.g., Thomas M. Ashton et al., Nature Communications 7:12308 (DOI: 10.1038/ncomms12308 |www.nature.com/naturecommunications) (referred to herein as “Ashton et al.”). Ashton et al. provides an assay (described below) for determining whether a compound is capable of reducing the cellular OCR.

One such OCR-API is Atovaquone, which is a naphthoquinone and analogue of ubiquinone. Atovaquone is approved by the FDA for the prevention and treatment of Pneumocystis jirovecii pneumonia (PCP). It also is prescribed by physicians to treat malaria, toxoplasmosis, and babesiosis. Atovaquone also is available in tablet form, often together with Proguanil hydrochloride for treatment and prophylaxis of malaria and PCP. Atovaquone has no known use or effect for treating mucositis. And as shown below, atovaquone by itself exhibits no protective effect for primary human gingival epithelial cells (HGEPp) cells from oxidative damage induced by hydrogen peroxide (H2O2), and does not change reactive oxygen species (ROS) levels in H2O2 challenged HGEPp cells.

### SUMMARY

As discussed herein, administering formulations of oltipraz can provide a beneficial, protective effect to the mucosal cells of a patient who is undergoing a treatment such as chemotherapy and/or radiation therapy (e.g., for treating head and neck cancer), which can damage the patient's mucosal cells. Administering oltipraz in combination with one or more OCR-APIs such as atovaquone can provide an enhanced benefit to the mucosal cells of such patients as compared to the benefit provided by administering oltipraz alone. As discussed herein, such OCR-APIs can be formulated together or separately from the oltipraz, and can be administered together with the oltipraz, or separately from the oltipraz.

As discussed herein, the properties of crystalline oltipraz can be improved by formulating compositions in which crystal parameters including particle size are controlled. Such compositions are described in PCT Application IB2017-001312, filed Sep. 12, 2017 (“Formulations of 4-Methyl-5-(Pyrazin-2-yl)-3H-1,2-Dithiole-3-Thione, and Methods of Making and Using Same”; Applicant ST IP Holding AG), the disclosure of which is incorporated herein by reference. By controlling the crystal particle size and formulation, crystals of oltipraz are provided that have prolonged size-stability in aqueous suspension and improved aqueous solubility as compared to previously known forms of oltipraz such as recrystallized oltipraz prepared according to the process disclosed in U.S. Pat. No. 4,110,450 or in PCT/IN2016/050197 to Framroze (published as WO 2016/207914 A2). For example, formulations comprising crystals of oltipraz that are of a controlled, much smaller size have beneficial properties such as excellent stability in the form of a dry composition and/or the ability to be readily re-suspended in aqueous compositions to form substantially homogenous dispersions of oltipraz crystals that typically exhibit substantially improved solubility, size-stability and/or efficacy compared to other forms of oltipraz known in the art. Further, unlike recrystallized oltipraz, the oltipraz-containing formulations disclosed herein can increase the gene expression of glutathione peroxidase 4 (GPX4) and/or myeloperoxidase (MPO) in a human or non-human animal patient, as well as decrease the gene expression of Peroxiredoxin 2 (PRDX2) in a human or non-human animal patient. Combining and/or administering such forms and compositions of oltipraz crystals together with OCR-API's thus provides therapeutic compositions that have therapeutic uses for example in the treatment of mucositis, and which provide enhanced protective effect for mucosal cells as compared to administering recrystallized oltipraz alone.

The crystals of oltipraz can have a MHD of from 30 to 2000 nm. As described in more detail below, the term ‘MHD’ is a measure of particle size and refers to the intensity averaged, mean hydrodynamic diameter (Z-average) as determined by the cumulants fitting of dynamic light scattering. Such crystals have improved solubility in aqueous solution compared to previous crystal forms of oltipraz and when comprised in pharmaceutical compositions provide for increased therapeutic efficacy.

As discussed herein, the crystals can have an intensity averaged, mean hydrodynamic diameter (Z-average) as determined by dynamic light scattering (DLS) in a range of from 30 to 2000 nm. (For convenience, in this disclosure the dimension of “intensity averaged, mean hydrodynamic diameter (Z-average) as determined by the cumulants fitting of dynamic light scattering” data is abbreviated as “MHD” and the precise method by which DLS measurements can be made to determine the MHD are provided below.) Usually, the crystals have a MHD of from 30 to 1200 nm; more often from 100 to 700 nm and still more typically from 150 to 450 nm or from 400 nm to 700 nm or from 400 nm to 600 nm. In certain embodiments, the crystals have a MHD within a target range of from 30 to 100, 100 to 1200 nm, 150 to 600 nm, 150 to 450 nm, 400 nm to 700 nm, 400 nm to 600 nm or 450 to 550 nm.

Oltipraz crystal compositions that may be used according to this disclosure typically comprise at least one stabilizing agent that stabilizes the crystals such that they retain a MHD within a target range of from 100 to 2000 nm if left in water at 25° C. for a period of from 1 to 24 hours, such as a period of 1 hour, 6 hours, or 24 hours. Usually, the stabilized crystals retain a MHD in a target range of 30 to 100, 100 to 1200 nm, 150 to 600 nm, 150 to 450 nm, 400 to 700 nm, 400 to 600 nm or 450 to 550 nm if left in water at 25° C. for a period of from about 1 to about 24 hours, such as about 6 hours. Usually, the stabilized crystals will retain a MHD in a target range of 30 to 100, 100 to 1200 nm, 150 to 600 nm, 150 to 450 nm, 400 to 700 nm, 400 to 600 nm, or 450 to 550 nm if left in water at 25° C. for a period of 1 hour, 6 hours, or 24 hours. Typically, the stabilizing agent is one or more of a polymer, a surfactant and/or a bulking agent. In certain embodiments, the crystals are stabilized by a combination of stabilizing agents such as a polymer and surfactant, which together act to stabilize the crystals.

OCR-APIs are known and include. e.g., meclizine, nimorazole. metformin. phenformin, antimycin A, pyrvinium, herberine, niclosamide, acriflavinium, sorafenib, emetine, plicamycin, suloctidil, pentamidine, amsacrine, irinotecan, itraconazole, mitomycin, hydroxyprogesterone, cyclosporine, fenofibrate, and analogues of ubiquinone such as atovaquone. See, e.g., Ashton et al.

This disclosure provides dry and liquid compositions comprising oltipraz and/or other Nrf2 activator and one or more OCR-APIs as described herein. The dry compositions can be mixed with water and/or another liquid to provide a liquid composition of oltipraz and/or other Nrf2 activator and the OCR-API. This disclosure also provides methods of making such dry and liquid suspensions. This disclosure also provides OCR-APIs in combination with dry oltipraz-containing compositions, including, e.g., spray-dried or lyophilized compositions, prepared from aqueous compositions comprising the crystals and a bulking agent. This disclosure also provides pharmaceutical compositions comprising OCR-APIs and such crystals. This disclosure further provides pharmaceutical containers for preparing and administering a dose of a liquid pharmaceutical composition comprising OCR-APIs and crystals as described herein. This disclosure also provides methods of treating human and non-human animal patients with pharmaceutical compositions disclosed herein comprising OCR-APIs and oltipraz. Further provided are compositions comprising OCR-APIs and oltipraz crystal compositions for use in the treatment of a patient such as a human or non-human animal. Such crystals are described herein, as well as compositions comprising OCR-APIs and such crystals for use in the manufacture of a medicament for the treatment of a patient in need thereof, such as a human or non-human animal.

Additionally, this disclosure provides methods of treating human and non-human animal patients with pharmaceutical compositions disclosed herein comprising oltipraz or formulated oltipraz crystal compositions and/or other Nrf2 activator as described herein, either with or without one or more OCR-APIs or other pharmaceutically active ingredients, for use in treating conditions where the patient can benefit from a reduced oxygen consumption rate, e.g., to prevent, treat, lessen the symptoms, and/or decrease the injury associated with ischemia/reperfusion injury. Such injury can occur, for example, during vascular repair procedures, myocardial infarction, a variety of vascular procedures in which a clot is removed, including stroke, and organ transplant surgery. The oltipaz or formulated oltipraz crystal compositions and/or other Nrf2 activators described herein can provide protection for the ischemic cells and/or protect the cells from oxidative damage when reperfusion is established.

### BRIEF DESCRIPTION OF THE FIGURES

FIGS. 1 and 2 are respectively a correlogram and an intensity size distribution for a DLS analysis of a sample of suspended crystals. The relaxation time is 1180 microseconds, Z-ave is 403 nm, and the PdI is 0.364.

FIG. 3a is a scanning electron microscopy (SEM) image at 5000× magnification of a composition comprising oltipraz described in Example 2 prior to stability testing.

FIG. 3b is a SEM image at 5000× magnification of the dry composition described in Example 2 after stability testing for three months at 40° C. and 75% RH.

FIG. 3c is a SEM image at 1500× magnification of the dry composition described in Example 2 after stability testing for three months at 40° C. and 75% RH.

FIG. 4A is a graph of the mean percentage of weight change in the oral mucositis assessment described in Example 3. FIG. 4B is a graph of the mean daily mucositis scores in the oral mucositis assessment described in Example 3.

FIG. 5 is a graph of the chi-square analysis of the percent of animal days with a mucositis score ≥3 in the oral mucositis assessment described in Example 3.

FIG. 6 is an illustration of the five aqueous suspensions described in Example 4 comprising formulated oltipraz compositions. FIG. 6 illustrates the effect of different bulking agents on the stability of the oltipraz crystals in an aqueous suspension.

FIG. 7 is a graph showing the effect of hydrogen peroxide (H2O2) on the viability of primary human gingival epithelial cells (HGEPp).

FIG. 8 is a graph showing the effect of recrystallized oltipraz, formulated oltipraz composition as described herein, and a control powder on H2O2-induced oxidative stress in HGEPp cells.

FIG. 9 is a graph showing the effect of recrystallized oltipraz and formulated oltipraz composition as described herein on the production of reactive oxygen species (ROS) in HGEPp cells.

FIG. 10 is a graph showing the effect of various concentrations of atovaquone alone, various concentrations of a formulated oltipraz crystal composition alone, and various concentrations of a combinations of atovaquone and the formulated oltipraz crystal composition on gene expression of oxidative stressed genes in peroxide-induced oxidatively stressed HGEPp cells.

FIG. 11 is a graph showing the effect of various concentrations of atovaquone alone, various concentrations of a formulated oltipraz crystal composition alone, and various concentrations of a combinations of atovaquone and the formulated oltipraz crystal composition on the production of reactive oxygen species (ROS) in HGEPp cells.

FIG. 12 is a graph showing the cell number by hoechst 33342 nucleic acid stain of positive and negative controls in Example 18. Neurons were incubated with MK801 10 μg/ml for one hour eighteen hours prior to the OGD induction. After treatments, HCS assay was performed. Data points represent the mean±SD for each condition. The results of the compounds were normalized according to the normoxia-treated cells.

FIG. 13 is a graph showing the cell number by hoechst 33342 nucleic acid stain in cells treated under OGD cytotoxicity condition in Example 18. Neurons were incubated with compounds for one hour eighteen hours prior to the OGD induction. After treatments, HCS assay was performed. Data points represent the mean t SD for each condition. The results of the compounds were normalized according to the normoxia-treated cells.

FIG. 14 is a graph showing Caspase 3 activation by CellEvent® Caspase-3n Green Detection Reagent of positive and negative controls in Example 18. Neurons were incubated with MK801 10 μg/ml for one hour eighteen hours prior to the OGD induction. After treatments. HCS assay was performed. Data points represent the mean±SD for each condition of number of apoptotic cells per nuclei.

FIG. 15 is a graph showing Caspase 3 activation by CellEvent® Caspase-3n Green Detection Reagent in cells treated under OGD condition in Example 18. Neurons were incubated with compounds for one hour eighteen hours prior to the OGD induction. After treatments, HCS assay was performed. Data points Data points represent the mean±SD for each condition of number of apoptotic cells per nuclei.

FIG. 16 is a graph showing neurite outgrowth by beta III tubulin staining of positive and negative controls in Example 18. Neurons were incubated with MK801 10 μg/ml for one hour eighteen hours prior to the OGD induction. After treatments, HCS assay was performed. Data points represent the mean±SD for each condition. The results of the compounds were normalized according to the normoxia-treated cells.

FIG. 17 is a graph showing neurite outgrowth by beta III tubulin staining in cells treated under OGD cytotoxicity condition in Example 18. Neurons were incubated with compounds for one hour eighteen hours prior to the OGD induction. After treatments. ICS assay was performed. Data points represent the mean±SD for each condition. The results of the compounds were normalized according to the normoxia-treated cells.

FIG. 18 is a graph showing LDH secretion by LDH Kit determination of positive and negative controls in Example 18. Neurons were incubated with MK801 10 μg/ml for one hour eighteen hours prior to the OGD induction. After treatments, HCS assay was performed. Data points represent the mean±SD for each condition. The results of the compounds were normalized according to the normoxia-treated cells.

FIG. 19 is a graph showing LDH secretion by LDH Kit determination in cells treated under OGD cytotoxicity conditions in Example 18. Neurons were incubated with compounds for one hour eighteen hours prior to the OGD induction. After treatments, HCS assay was performed. Data points represent the mean SD for each condition. The results of the compounds were normalized according to the normoxia-treated cells.

FIG. 20 is a graph showing mitochondrial damage by TMRM dye of positive and negative controls in Example 18. Neurons were incubated with MK801 10 μg/ml for one hour eighteen hours prior to the OGD induction. After treatments, HCS assay was performed. Data points represent the mean±SD for each condition. The results of the compounds were normalized according to the normoxia-treated cells

FIG. 21 is a graph showing mitochondrial damage by TMRM dye in cells treated under OGD cytotoxicity condition in Example 18. Neurons were incubated with compounds for one hour eighteen hours prior to the OGD induction. After treatments, HCS assay was performed. Data points represent the mean t SD for each condition. The results of the compounds were normalized according to the normoxia-treated cells.

FIG. 22 is a graph showing cell viability by WST8 dye of positive and negative controls in Example 18. Neurons were incubated with MK801 10 μg/ml for one hour eighteen hours prior to the OGD induction. After treatments, HCS assay was performed. Data points represent the mean±SD for each condition. The results of the compounds were normalized according to the normoxia-treated cells.

FIG. 23 is a graph showing Cell viability by WST8 dye in cells treated under OGD citotoxicity condition in Example 18. Neurons were incubated with compounds for one hour eighteen hours prior to the OGD induction. After treatments. HCS assay was performed. Data points represent the mean±SD for each condition. The results of the compounds were normalized according to the normoxia-treated cells.

FIG. 24 is a graph showing cell viability by WST8 dye of positive and negative controls for Example 19. Cardiomyocytes were incubated with NAC during 4 hours prior to the OGD induction and was also present during the OGD insult and 24 h-recovery period. After treatments, WST-8 assay was performed. Data points represent the mean t SD for each condition. The results of the compounds were normalized according to the normoxia-treated cells.

FIG. 25 is a graph showing cell viability by WST8 dye in cells treated under OGD cytotoxicity condition in connection with Example 19. Cardiomyocytes were incubated with compounds during 4 hours prior to the OGD induction and were also present during the OGD insult and 24 h-recovery period. After treatments. HCS assay was performed. Data points represent the mean±SD for each condition. The results of the compounds were normalized according to the normoxia-treated cells.

FIG. 26 is a graph showing cell numbers by hoechst 33342 nucleic acid stain of positive and negative controls in connection with Example 19. Cardiomyocytes were incubated with NAC during 4 hours prior to the OGD induction and was also present during the OGD insult and 24 h-recovery period. After treatments, HCS assay was performed. Data points represent the mean±SD for each condition. The results of the compounds were normalized according to the normoxia-treated cells.

FIG. 27 is a graph showing cell number by hoechst 33342 nucleic acid stain in cells treated under OGD cytotoxicity condition in connection with Example 19. Cardiomyocytes were incubated with compounds during 4 hours prior to the OGD induction and were also present during the OGD insult and 24 h-recovery period. After treatments, HCS assay was performed. Data points represent the mean t SD for each condition. The results of the compounds were normalized according to the normoxia-treated cells.

FIG. 28 is a graph showing Caspase 3n activation by CellEvent® Caspase-3n Green Detection Reagent of positive and negative controls in connection with Example 19. Cardiomyocytes were incubated with NAC during 4 hours prior to the OGD induction and was also present during the OGD insult and 24 h-recovery period. After treatments, HCS assay was performed. Data points represent the mean t SD for each condition of number of apoptotic cells per nuclei.

FIG. 29 is a graph showing FIG. 4. Caspase 3n activation by CellEvent® Caspase-3n Green Detection Reagent in cells treated under OGD condition in connection with Example 19. Cardiomyocytes were incubated with compounds during 4 hours prior to the OGD induction and were also present during the OGD insult and 24 h-recovery period. After treatments, HCS assay was performed. Data points Data points represent the mean±SD for each condition of number of apoptotic cells per nuclei.

FIG. 30 is a graph showing LDH secretion by LDH Kit determination of positive and negative controls in connection with Example 19. Cardiomyocytes were incubated with NAC during 4 hours prior to the OGD induction and was also present during the OGD insult and 24 h-recovery period. After treatments, HCS assay was performed. Data points represent the mean±SD for each condition. The results of the compounds were normalized according to the normoxia-treated cells.

FIG. 31 is a graph showing LDH secretion by LDH Kit determination in cells treated under OGD citotoxicity condition in connection with Example 19. Cardiomyocytes were incubated with compounds during 4 hours prior to the OGD induction and were also present during the OGD insult and 24 h-recovery period. After treatments. HCS assay was performed. Data points represent the mean±SD for each condition. The results of the compounds were normalized according to the normoxia-treated cells.

FIG. 32 is a graph showing intracellular ATP by Luminescence ATP Detection Assay Kit of positive and negative controls in connection with Example 19. Cardiomyocytes were incubated with NAC during 4 hours prior to the OGD induction and was also present during the OGD insult and 24 h-recovery period. After treatments, HCS assay was performed. Data points represent the mean±SD for each condition. The results of the compounds were normalized according to the normoxia-treated cells.

FIG. 33 is a graph showing intracellular ATP by Luminescence ATP Detection Assay Kit in cells treated under OGD citotoxicity condition in connection with Example 19. Cardiomyocytes were incubated with compounds during 4 hours prior to the OGD induction and were also present during the OGD insult and 24 h-recovery period. After treatments, HCS assay was performed. Data points represent the mean±SD for each condition. The results of the compounds were normalized according to the normoxia-treated cells.

### DETAILED DESCRIPTION

As discussed above, one aspect of this disclosure relates to the administration of an OCR-API such as atovaquone in combination with oltipraz, e.g., for the prophylactic and/or therapeutic treatment of mucosal cells in a patient who will undergo, or is undergoing, a treatment such as chemotherapy and/or radiation therapy that places stress on the patient's mucosal cells. Another aspect of this disclosure relates to the administration of (i) oltipraz or formulated oltipraz crystal compositions and/or other Nrf2 activator as described herein, either with or without one or more OCR-APIs or other pharmaceutically active ingredients, or (ii) an OCR-API such as atovaquone in combination with oltipraz and/or other Nrf2 activator(s), e.g., for use in treating a patient having a condition where the patient can benefit from a reduced oxygen consumption rate. e.g., to prevent, treat, lessen the symptoms, and/or decrease the injury associated with ischemia/reperfusion injury. Such injury can occur, for example, during vascular repair procedures, myocardial infarction, a variety of vascular procedures in which a clot is removed, including stroke, and organ transplant surgery. The oltipraz can be any form of oltipraz, including recrystallized oltipraz such as that disclosed in U.S. Pat. No. 4,110,450 or Framroze PCT/IN2016/050197, mentioned above. Alternatively, the oltipraz such as that produced by Framroze PCT/IN2016/050197 may be further processed and formulated into oltipraz crystal-containing compositions as described in Section A below for use in the products and processes of this disclosure.

A. Processed and Formulated Compositions Comprising Crystals of 4-Methyl-5-(Pyrazin-2-yl)-3H-1,2-Dithiole-3-Thione

As noted above, the compositions and methods of this disclosure can include the processed and formulated compositions comprising oltipraz crystals described in PCT Application IB2017-001312 (“Formulations of 4-Methyl-5-(Pyrazin-2-yl)-3H-1,2-Dithiole-3-Thione, and Methods of Making and Using Same”; Applicant ST IP Holding AG), which describes oltipraz crystals having an MHD in the range of from 30 to 2000 nm, such as from 30 to 1200 nm, e.g. 100 to 600 nm, 400 to 700 nm, 400 to 600 nm, preferably 150 to 450 nm, 400 to 700 nm, 400 to 600 nm or 450 to 550 nm. Because such processed and formulated compositions comprising oltipraz crystals may be used in accordance with the compositions and methods of this disclosure, they are described herein in this Section A.

Certain embodiments of the compositions and methods described herein comprise a quantity of crystals of 4-methyl-5-(pyrazin-2-yl)-3H-1,2-dithiole-3-thione having an MHD in the range of from 30 to 100, 100 to 200 nm, with embodiments having MHD's within target ranges of from 30 to 100, 100 to 1200 nm, 150 to 600 nm, 150 to 450 nm, 400 to 700 nm, 400 to 600 nm or 450 to 550 nm. The MHD of the crystals may be measured in any number of ways known to skilled artisans, including dynamic light scattering as described herein. As mentioned above, the oltipraz can be prepared in crystalline form. Embodiments of the oltipraz crystal compositions provided herein have been found to provide dry compositions of oltipraz crystals that are stable for extended periods, and which are able to be readily re-suspended in aqueous compositions to form substantially homogeneous dispersions of oltipraz crystals that exhibit substantially improved properties as compared to the previously available crystalline form.

Oltipraz crystal compositions that may be used in the products and processes described herein typically also exhibit substantially increased rate of dissolution and solubility in water e.g., at 20° C. as compared to oltipraz crystals prepared from standard methods (e.g., ranging from 20 μm to 200 μm or greater). For example, the oltipraz crystals in the compositions of this disclosure typically have a solubility in water at 20° C. between 10 and about 250% that of crystals of oltipraz, prepared from recrystallization and having diameters of 20 to 200 μm. More typically, oltipraz crystals useful in the compositions and processes of this disclosure have a solubility of from about 130 to about 220%, such as from about 160% to about 200% e.g. from about 170 to about 190% that of oltipraz crystals of 20 to 200 μm in diameter.

As discussed in Example 5 below, the solubility of oltipraz in water at 20° C. in certain embodiments of compositions disclosed herein is almost double that of the larger oltipraz crystals (e.g., a >80% increase). Solubility values of >3.5 μg/ml, >4.0 μg/ml. >4.5 μg/ml, >5.0 μg/ml and >5.5 μg/ml are all possible, including. e.g., about 5.1 μg/ml, about 5.2 μg/ml, about 5.3 μg/ml, about 5.4 μg/ml, about 5.5 μg/ml, about 5.6 μg/ml, and about 5.7 μg/ml. Hence, solubility values in water at 20° C. in the following exemplary ranges are possible: 3.5 μg/ml to 8.0 μg/ml, 3.5 μg/ml to 7.0 μg/ml, 3.5 μg/ml to 6.0 μg/ml, 3.5 μg/ml to 5.7 μg/ml, 4.0 μg/ml to 8.0 μg/ml, 4.0 μg/ml to 7.0 μg/ml, 4.0 μg/ml to 6 μg/ml, 4.0 μg/ml to 5.7 μg/ml, 4.5 μg/ml to 8.0 μg/ml, 4.5 μg/ml to 7.0 μg/ml, 4.5 μg/ml to 6.0 μg/ml, 4.5 μg/ml to 5.7 μg/ml, 5.0 μg/ml to 8.0 μg/ml, 5.0 μg/ml to 7.0 μg/ml, 5.0 μg/ml to 6.0 μg/ml, 5.0 μg/ml to 5.7 μg/ml, 5.5 μg/ml to 8.0 μg/ml, 5.5 μg/ml to 7.0 μg/ml, 5.5 μg/ml to 6.0 μg/ml, 5.5 μg/ml to 5.7 μg/ml, 6.0 μg/ml to 6.5 μg/ml, 6.0 μg/ml to 7.0 μg/ml, 6.0 μg/ml to 8.0 μg/ml, 6.5 μg/ml to 7 μg/ml, 6.5 μg/ml to 8.0 μg/ml, 7 μg/ml to 8.0 μg/ml, and greater than 8.0 μg/ml.

Typically, therefore, the oltipraz crystal compositions that are described in this Section A. and which can be used in combination with the OCR-API, will have a solubility in water at 20° C. of from about 3.5 to about 8 μg/ml, more typically from about 4 to about 7.5 μg/ml, such as from about 4.5 to about 7 μg/ml e.g. from about 5 to about 6.5 μg/ml such as from about 5.5 to about 6 μg/ml, e.g. about 5.7 μg/ml.

The oltipraz crystals described in this Section A can be prepared by processing oltipraz into crystals having the desired size range using processes as described below. In some circumstances, once the desired size is attained, however, the crystals in aqueous or other liquid solution will tend to grow larger over time. e.g., by agglomerating and/or recrystallizing to form larger crystals. Hence, in instances where it is desired to prevent the crystals from growing larger for a period of time, at least one stabilizing agent may be added to the composition in order to help maintain the crystals in the desired size range in the liquid solution.

Typically, the stabilizing agent is a polymer, which may be used alone or in combination with one or more other stabilizing agents such as surfactants, to stabilize the individual crystals by inhibiting and/or preventing, for at least a period of time, the formation of larger crystals. e.g., through agglomeration, ripening (e.g. Ostwald ripening), and/or recrystallization. In certain embodiments, the polymer can be a polymer that comprises charged moieties. In other embodiments, the polymer may be neutral. Sometimes, one or more surfactants may be employed as stabilizing agents, either alone or together with a polymer. Various polymers and/or surface-active molecules can have an affinity for the oltipraz crystal surface, e.g., such that they can coat, adsorb, adhere or otherwise associate with all or a portion of the crystals and thereby interfere with the crystals agglomerating, ripening, and/or recrystallizing to form larger crystals.

As noted above, the quantity of crystals in the liquid suspension then may be further treated to produce a dry composition, e.g., by mixing a bulking agent with a liquid composition of crystals and then removing the liquid from the composition to form a dry composition. e.g., by spray-drying or lyophilizing an aqueous composition. The bulking agent can also serve as a stabilizing agent, either alone or in combination with other stabilizing agents. When a bulking agent is used, the dry composition thus will comprise both the crystalline oltipraz drug and the bulking agent, as well as any other stabilizing agents or other ingredients that are present in the liquid composition prior to the removal of the water and/or other liquid solvent. When the dry composition is then mixed with liquid (e.g., water), the drug crystals and other ingredients present in the dry composition will then be released into the liquid.

The term “dry composition” as used herein refers to a composition that substantially excludes water or other solvent. As used in this disclosure, the term “substantially” is intended to encompass both “wholly” and “largely but not wholly.” Thus, a dry composition that substantially excludes water is a composition that wholly excludes water (and/or other solvent) or largely excludes water (and/or other solvent). That is, the dry composition either has no water or solvent, or at most only a small or residual amount of water or solvent such that the composition is not moist or wet.

1. Liquid Compositions Comprising Crystals in Suspension

Any suitable method can be used to produce the oltipraz crystals of this Section A. For example, recrystallized oltipraz crystals such as those produced by U.S. Pat. No. 4,110,450 or Framroze PCT/IN2016/050197 can be wet milled in the presence of at least one stabilizing agent that can help to stabilize the drug crystals to reduce or prevent the growth of crystals by agglomeration, ripening and/or recrystallization. The wet milling of oltipraz crystals in the presence of the stabilizing agent thus creates a liquid (e.g., aqueous) composition comprising the oltipraz crystals in suspension in the composition. Combinations of stabilizing agents may be added to the wet milling composition to facilitate stabilization of the crystals.

As an alternative to wet-milling, oltipraz crystals useful for compositions of this Section A may be made by other method of producing nanocrystals, e.g., by antisolvent precipitation, supercritical fluid precipitation, printing techniques adapted from the semiconductor industry, or three-dimensional printing or other known means of producing nanoparticles.

For example, a liquid composition comprising at least a portion of the crystals as described in this Section A and optionally other additives (the crystals having been prepared. e.g., from a wet milling or antisolvent process), can be admixed with a bulking agent to form a liquid composition comprising the bulking agent and crystals in suspension. In certain embodiments, a liquid composition comprising at least a portion of the crystals and other additives. e.g., from a wet milling or antisolvent process, is then admixed with a bulking agent to form a liquid composition comprising the bulking agent and crystals in suspension. That liquid composition then may be processed to remove the liquid, e.g., by spray-drying or lyophilization in the case of aqueous solutions, and additional drying if necessary, to form a dry composition that substantially excludes water. Other processes known to persons skilled in the art also may be used to prepare dry compositions comprising the crystals. For example, the liquid composition can be sprayed onto sugar spheres or beads for drying. When dry, the sugar spheres or beads become a dissolvable carrier for the drug and other additives, e.g., the stabilizing agent(s) and/or bulking agent(s). The dry composition thus comprises the oltipraz crystals and any ingredients other than the liquid solvent (e.g., water) that were present in the liquid composition. The dry composition can be then later admixed with a liquid comprising water, at which time the bulking agent can facilitate release of the crystals to again form an aqueous composition comprising such crystals in suspension. Any additional nonvolatile ingredients present in the liquid composition prior to removal of water or other solvent will be carried along in the dry composition and also released into the re-suspended aqueous composition.

Depending on the amount of water and/or other liquid solvent used in the milling or other nanocrystal production process such as antisolvent precipitation, the oltipraz crystals can be present in an amount ranging from 2% or less to 40% or more by weight of the liquid composition prior to the addition of any bulking agent. Within that range are included the following ranges in percent by weight of 1-20%, 2 to 5%, 5 to 10%, 10 to 15%, 10 to 20%, 15 to 20%, 15 to 25%, 15 to 30%, 20 to 30%, 25 to 35%, 30 to 40%, or more than 40%. In some embodiments, the crystals can be between 6 and 11% by weight of the liquid composition, e.g., between 7 and 10%. In certain such embodiments, the concentration of the crystals in the liquid is about 1% to about 30% by weight, about 4% to about 15% by weight, about 5% to about 10% by weight, about 6% to about 10% by weight, about 6% to about 12% by weight, about 7% to about 10% by weight, about 8% to about 10% by weight, or about 8.6% by weight of the suspension. Accordingly, the liquid composition typically comprises between about 1 to about 40 wt %, such as from about 2 to about 20 wt %. e.g. from about 4 to about 15 wt %, typically from about 6 to about 12 wt % such as from about 7 to about 10 wt % e.g. about 8 to about 9 wt % such as about 8.6 wt % of oltipraz crystals, based on the weight of the liquid composition prior to the addition of any bulking agent.

Alternatively, the amount of crystals can be calculated as a percent of the components other than the water or other liquid solvent in the composition prior to addition of a bulking agent. As a percent of the non-solvent components, the crystals can be present in an amount ranging less than 10% up to more than 60% by weight of the non-solvent components prior to the addition of any bulking agent. Within that range are included the following ranges in percent by weight of 1 to 5%, 5 to 10%, 10 to 15%, 15 to 20%, 20 to 30%, 25 to 40%, 30 to 40%, 40 to 50%, 50 to 60%, 60 to 70%, and over 70%. In certain embodiments, the crystals comprise between 30 and 70%, e.g., between 50 and 65%, or between 55 and 60%, or about 57% by weight of the non-solvent components prior to addition of any bulking agent. Accordingly, the non-solvent components in the composition typically comprise from about 1 to about 70 wt % oltipraz crystals based on the overall weight of the non-solvent components in the composition; more typically the non-solvent components comprise from about 30 to about 65 wt % such as from about 50 to about 60 wt % e.g. from about 55 to about 58 wt % such as about 57 wt % of the composition based on the overall weight of the non-solvent components in the composition.

Once a bulking agent is added, the percentage by weight of the oltipraz crystals typically will decrease. Within the liquid composition before removal of water or other liquid solvent but after addition of the bulking agent, the crystals may comprise from 1% up to 10% or more of the liquid composition. Within such ranges are, e.g., 1 to 2%, 1 to 3%, 2 to 3%, 2 to 4%, 2 to 5%, 2 to 6%, 3 to 5%, 3 to 6%, 3 to 7%, 4 to 7%, 4 to 8%, 5 to 9% and 6 to 10%. In some embodiments, the crystals can comprise between 2 and 6% of the liquid suspension comprising the bulking agent. e.g., between 3 and 5%, or about 4%. In certain such embodiments, the concentration of the crystals in the liquid is about 0.1% to about 4% by weight, about 0.2% to about 3.5% by weight, about 0.5% to about 3.5% by weight, about 1% to about 3.5% by weight, about 1.5% to about 3% by weight, about 2% to about 3% by weight, or about 2.5% by weight of the formulation. Accordingly, the concentration of oltipraz crystals in the liquid is typically from about 0.1 to about 10 wt % (based on the weight of the liquid composition before removal of water or other liquid solvent but after addition of a bulking agent if present), more often from about 0.5 to about 8 wt % e.g. from about 1 to about 6 wt % such as from about 2 wt % to about 5 wt % such as from about 2.5 wt % to about 4 wt %.

Alternatively, the amount of the crystals can be calculated as a percent of the non-solvent (e.g., non-water) components following addition of a bulking agent. This percentage of oltipraz in the non-solvent components also may be referred to as the “drug loading” percentage because it represents the amount of the oltipraz crystals in the dry composition. As a percent of the non-solvent components, i.e., the solids, the oltipraz crystals can be present in an amount ranging from less than 2% up to 25% or more. Within that range are included the following ranges in percent by weight of 0.5 to 1%, 1% to 2%, 2 to 4%, 3 to 5%, 4 to 7%, 5 to 8%, 5 to 10%, 6 to 8%, 6 to 9%, 6 to 10%, 7 to 11%, 7 to 12%, 8 to 12%, 8 to 13%, 9 to 13%, 9 to 14%, 10 to 15%, 11 to 16%, 12 to 17%, 13 to 18%, 14 to 19%, 15 to 20% and 20 to 25%. Accordingly, the oltipraz crystals are typically from about 0.5 to about 25 wt % (based on the weight of the non-solvent components after addition of a bulking agent if present), more often from about 1 to about 25 wt % such as from about 5 to about 20 wt % e.g. from about 6 to about 19 wt %, such as from about 10 to about 18 wt % e.g. about 15 to about 17 wt % such as about 16 wt % (e.g. about 16.7 wt %). The crystals can comprise between about 5% and about 10% by weight of the non-solvent components. e.g., between about 6% and about 9%, such as about 7%. For example, in certain embodiments the crystals can comprise between 5 and 10% by weight of the non-solvent components, e.g., between 6 and 9%, or about 7%. In other embodiments the crystals comprise between 10 and 20% by weight of the powder, e.g., between 13 and 17%, e.g., about 15% by weight of the non-solvent components.

In some embodiments of the oltipraz compositions of this Section A, a dry composition comprising a oltipraz drug loading of about 15% will provide good results when reconstituted with water, i.e., the dry composition quickly forms a dispersion (e.g., less than a minute) with moderate or gentle shaking, with the crystals substantially retaining their MHD from prior to drying. Typically, a dry composition comprising an oltipraz drug loading of about 20% or higher provides less desirable results when reconstituted with water, i.e., the dry composition slowly forms a dispersion (e.g., several minutes) with moderate or vigorous shaking, and the dispersion may comprise larger particles, e.g., up to 2 microns in size. In such cases, it is advantageous to reduce the oltipraz drug loading to a lower level that provides the desired characteristics in terms of rapidly forming a dispersion of crystals that retain their original MHD. Without being bound by any particular theory, it is believed that as the concentration of oltipraz crystals within the dry composition approaches 20%, there is less of the other ingredients in the composition (e.g., stabilizing agents and/or bulking agents) to separate the individual crystals, which in turn leads to more interactions between the crystals, resulting in slower formation of a dispersion in an aqueous or other solvent environment and also the formation of larger particles. e.g., by agglomeration. Hence, compositions of this Section A comprising oltipraz drug loadings of 12 to 20% are contemplated, including loadings of 12 to 13%, 12 to 14%, 12 to 15%, 13 to 14%, 13 to 15%, 13 to 16%, 14 to 15%, 14 to 16%, 14 to 17% 15 to 16%, 15 to 17%, 15 to 18%, 16 to 17%, 16 to 18%, 16 to 19%, 17 to 18%, 17 to 19%, 17 to 20%, 18 to 19%, 18 to 20%, including drug loadings of about 12%, about 13%, about 14%, about 15%, about 16%, about 17%, about 18%, about 19% and about 20% are all contemplated. Accordingly, the dry composition typically has an oltipraz drug loading of about 12 to about 20 wt % such as from about 14 to about 18 wt % e.g. from about 15 to about 17 wt % such as about 16 or about 16.7 wt %.

A. Stabilizing Agents

As mentioned above, liquid compositions comprising the oltipraz crystals of this Section A typically also comprise one or more stabilizing agents to stabilize the crystals. In some circumstances, in the absence of at least one stabilizing agent (or a combination of agents that together act to stabilize), over time oltipraz crystals in liquid suspension can agglomerate, ripen, and/or recrystallize to form larger crystals. It is typically desirable to maintain the crystals in the size range that results from the wet milling, antisolvent precipitation or other crystal-production processes for a period of time, e.g., to permit storage of the materials prior to the next step in processing, or to allow testing or validation of crystal size or some other feature of a batch of oltipraz crystals. In such instances, at least one stabilizing agent may be provided to the liquid composition of crystals, e.g., during and/or after milling, or during and/or after antisolvent precipitation, in order to stabilize the crystals to thereby prevent and/or inhibit the milled crystals from agglomerating, recrystallizing and/or ripening to form larger crystals. Thus, any agent that either alone or in combination with another agent serves to stabilize the crystals to thereby prevent and/or inhibit the milled crystals from agglomerating, recrystallizing and/or ripening to form larger crystals, is deemed a stabilizing agent. If a combination of two or more agents is used to stabilize crystals, then each of the two or more co-stabilizers is deemed to be a stabilizing agent even though an individual agent within the combination may be unable to stabilize the crystals by itself, or unable to stabilize the crystals by itself for the desired length of time.

Alternatively, if the oltipraz crystals of this Section A are to be quickly converted to a dry form, e.g., by mixing with a bulking agent and being spray-dried or lyophilized, a stabilizing agent may be unnecessary. This may be an acceptable alternative if the intended method of administration does not require the oltipraz crystals to later have stability upon resuspension in water, e.g., if the resuspension will occur immediately before administration of the dry composition, e.g., in pill or tablet form. Alternatively, a single agent such as copovidone or PVP-VA64 (polyvinylpyrrolidone vinyl acetate, a copolymer of 1-vinyl-2-pyrrolidone and vinyl acetate in a ratio of 6:4 by mass, commercially available e.g. from BASF as Product No. 95405-2-43), may be able to serve both as a stabilizing agent and as bulking agent, thereby rendering additional stabilizing agents unnecessary and providing a composition that will exhibit stability upon resuspension in water and/or other liquid.

Generally speaking, stabilizing agents are surface active agents that affect the surface of the crystals in some way. While not wishing to be bound to a particular theory by which a stabilizing agent can operate to stabilize the crystals, it is believed that the stabilization typically can take one of two forms. Steric stabilization can be accomplished by mixing the crystals with either an amphiphilic or water-soluble material that interacts with the crystal surface, which keeps crystal faces from interacting by providing a barrier between crystals. This is typically accomplished by addition of polymer, surfactant, or both. Alternatively, electrostatic stabilization can be accomplished by modifying the crystal surface with a charge through addition of a charged compound (polymer, surfactant, or other interacting charged molecule or ion). Because all or at least many of the crystals then carry the same charge, in theory they repel each other, thereby increasing the energy barrier required for two crystal faces to get close enough to fuse together.

Typically, the stabilizing agent maintains the size of the crystals in the liquid composition within a specified size range for a period of time following wet milling. Such a period can be on the order of hours, e.g., at least 1 hour, at least 6 hours, at least 12 hours, at least 24 hours, at least two days, at least three days, at least a week, at least two weeks, at least a month, at least two months, and at least six months, or longer.

Typically, the stabilizing agent comprises a polymer that is either neutral or capable of associating charged moieties with the individual milled oltipraz crystals, e.g., by coating the crystals, or adsorbing or otherwise associating with them. Such polymers thus may be neutral or may include moieties that provide either a positive or negative charge to the polymer, and in that way the charged moieties associated with the crystals may be able to repel other crystals having like charges on their surfaces. Nonionic, cationic or anionic polymers may be used as stabilizing agents, including especially pharmaceutically acceptable nonionic, cationic and anionic polymers. Combinations of such polymers also may be employed. Sometimes, the stabilizing agent may comprise a carbohydrate and/or protein, e.g., albumin.

The polymer may be an acrylate polymer comprised of a plurality of repeat units derived from identical or different monomers. Acrylate polymers comprising different types of repeat units are referred to herein as “copolymers”. Exemplary repeat units of acrylate polymers include repeat units derived from methacrylate, alkyl acrylate (such as methyl acrylate or ethyl acrylate), hydroxyethyl methacrylate, ethylacrylate, butyl methacrylate, acrylonitrile, or alkyl cyanoacrylates. Typically, when the carboxylic acid functionality of acrylate is not protected as an ester, the acid can exist as a protonated carboxylic acid (—COOH) or as an anionic salt (e.g., —COONa).

The polymer also may be an acrylate- and alkenyl ether-based co-polymer (e.g., Carbopol® type polymers such as Carbopol 974P NF), polyvinylpyrrolidine (e.g., PVP K15 or K30), a cellulosic polymer such as a cationic hydroxyethyl cellulose (e.g., in the Polymer JR family), hydroxypropylcellulose (HPC e.g. HPC EF typically having a molecular weight of about 80 kDa), or hydroxypropyl methylcellulose (HPMC e.g. HMPC E3 typically having viscosity of about 3 cP at 2% in water), or hydroxypropyl methylcellulose acetate succinate, HPMCAS. The polymer also may be a copovidone (e.g., PVP-VA64), poly(ethylene oxide), or a poloxamer (e.g., a poly(propylene oxide) and poly(ethylene oxide) copolymer). The polymer also may be an acrylamide polymer. For example, the polymer may be comprised of repeat units derived from acrylamide.

The repeat units can be functionalized by adding groups that can change the permeability, hydrophobicity, or other properties of the formulation. For example, certain repeat units can be functionalized by tertiary amines or by quaternary amines, such as quaternary trialkylammonium substituents.

An acrylate polymer may be comprised of repeat units derived from a methacrylate monomer. In certain embodiments, the acrylate polymer comprises repeat units derived from an acrylate monomer and repeat units derived from a methacrylate monomer. Typically, the acrylate polymer comprises repeat units derived from ethyl acrylate and repeat units derived from methyl methacrylate. Typically, some of the ethyl acrylate monomeric units are functionalized on the ethyl group by a trimethylammonium chloride group. The acrylate polymer of the crystal may be poly(ethyl acrylate-co-methyl methacrylate-co-trimethylammonioethyl methacrylate chloride) 1:2:0.2. Poly(ethyl acrylate-co-methyl methacrylate-co-trimethylammonioethyl methacrylate chloride) 1:2:0.2 may be sold as EUDRAGIT® RL Other polymethacrylate-based copolymers in the Eudragit family may be used, e.g., Eudragit S, L, E or RS.

Typically, the polymer is one or mom of an acrylate- and alkenyl ether-based co-polymer, polyvinylpyrrolidone, hydroxypropylcellulose, hydroxypropyl methylcellulose, a copovidone such as PVP-VA64, and a polymethacrylate-based copolymer such as EUDRAGIT® RL. More often, the polymer is one or more of a copovidone such as PVP-VA64 and a polymethacrylate-based copolymer such as EUDRAGIT® RL.

Alternatively, or in addition to the above polymers, other surface-active ingredients may be added to the liquid compositions that comprise the crystals for the purpose of helping to stabilize the crystals in suspension. In addition to helping stabilize the crystals such surfactants also may aid in the dispersion of crystals and/or other ingredients in a particular liquid composition. Indeed, such surfactants may be added solely for the purpose of aiding in the dispersion of crystals and/or other ingredients in the liquid compositions described herein that are prepared from the dry compositions described herein.

Surfactants suitable for use in the compositions described herein may be ionic or non-ionic. These include, but are not limited to: sodium isostearate, cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol. N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, dicetyl phosphate, ceteth-10 phosphate (ceteth-10 is the polyethylene glycol ether of cetyl alcohol where n has an average value of 10: ceteth-10 phosphate is a mixture of phosphoric acid esters of ceteth-10), ceteth-20, Brij S10 (polyethylene glycol octadecyl ether, average Mₙ˜711), PEG-20 phytosterol, and Poloxamers (including, but not limited to. Poloxamer 188 (HO(C₂H₄O)ₐ(CH(CH₃)CH₂O)_{b}, (C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamer surfactants, also sold under the trade name of Pluronic surfactants, thus may be employed, e.g., Pluronic F-68, which also is known as Poloxamer 188. The surfactants that may be used in the formulation may be non-ionic surfactants such as polyoxyethylene glycol alkyl ethers (e.g., octaethylene glycol monododecyl ether, pentaethylene glycol monododecyl ether, and polyethylene glycol alkyl ethers such as Brij® Detergents), polyoxypropylene glycol alkyl ethers, glucoside alkyl ethers (e.g., decyl glucoside, lauryl glucoside, or octyl glucoside), polyoxyethylene glycol alkylphenol ethers (e.g. Triton X-100. Nonoxyol-9), glycerol alkyl esters, polyoxyethylene glycol sorbitan alkyl esters (e.g., polysorbates), sorbitan alkyl esters, cocamides, and Poloxamers (mentioned above). In certain embodiments, the non-ionic surfactant may be polyoxyethylene (20) sorbitan monooleate (polysorbate 80). Polysorbate 80 is available under the tradename Tween 80.

Typically, the surfactant is one or more of poloxamers such as Pluronic F-68 (i.e., Poloxamer 188), polysorbates such as polysorbate 80 (Tween 80), povidone-based polymers, lecithin. PEG-castor oil derivatives, TPGS, bile acids, tyloxapol, acacia, and sodium lauryl sulfate. More typically, the surfactant is polysorbate 80 (Tween 80).

As described in more detail below, appropriate combinations or mixtures of surfactants such as those above may also be used, either with or without out other stabilizing agents such as the polymers described above. For example, in certain embodiments the stabilizing agents can comprise a combination of a neutral polymer and a neutral surfactant, a cationic polymer and a neutral surfactant, or a neutral polymer and an anionic surfactant. As noted above, however, such stabilizing agents may be unnecessary when the bulking agent also acts as a stabilizing agent or when no stabilizing agent is desired.

When such stabilizing agents are employed, then depending on the amount of water and/or other solvent used in the milling process, the stabilizing agent(s) can be present in an amount ranging from less than 1 percent to 25% or more by weight of the liquid composition of this Section A prior to the addition of any bulking agent. Within that range are included the following ranges in percent by weight of 0.1 to 1%, 1 to 3%, 3 to 7%, 5 to 10%, 5 to 15%, 5 to 20%, 10 to 15%, 10 to 20%, 10 to 25%, 15 to 20%, 15 to 25%, 7.5 to 25%, or more than 25%. In some embodiments, the stabilizing agent(s) can comprise between 2 and 10%, e.g., between 4 and 8% or about 6.4%. Accordingly, the amount of stabilizing agent(s) in the liquid composition of this Section A prior to addition of any bulking agent is typically from about 0.1 to about 25 wt % such as from about 1 to about 20 wt % such as from about 2 to about 10 wt % e.g. from about 4 to about 8 wt % such as from about 5 to about 7 wt % e.g. about 6 wt % such as about 6.4 wt %.

Alternatively, the amount of stabilizing agent can be calculated as a percent of the non-solvent components prior to addition of a bulking agent. As a percent of the non-solvent components, the stabilizing agent can be present in an amount ranging from 10 percent or less to 75% or more by weight of the non-liquid components prior to the addition of any bulking agent. Within that range are included the following ranges in percent by weight of 0.1 to 10%, 10 to 20%, 20 to 30%, 30 to 40%, 40 to 50%, 50 to 60%, 60 to 75% or more. In some embodiments, the stabilizing agent can be between 30 and 55% by weight of the non-solvent components. e.g., between 35 and 50%, or between 40 and 45%, or about 42.7%. Accordingly, the amount of stabilizing agent(s) in the composition prior to addition of any bulking agent is typically from about 1 to about 75 wt %, such as from about 10 to about 60 wt % such as from about 20 to about 55 wt % e.g. from about 30 to about 50 wt % such as from about 40 to about 45 wt % e.g. about 42 wt % such as about 42.7 wt %, based on the weight of non-solvent components.

Once a bulking agent is added, the percentage by weight of the stabilizing agent(s) typically will decrease. Within the liquid composition before removal of water and/or other liquid solvent, following addition of a bulking agent the stabilizing agent(s) may comprise from less than 1% up to 30% or more of the liquid composition, again depending on the amount of water or other solvent in the composition prior to a water removal step. Within such ranges are, e.g., 0.5 to 1%, 1 to 2%, 1 to 3%, 2 to 3%, 2 to 4%, 2 to 5%, 3 to 6%, 3 to 7%, 4 to 7%, 4 to 8%, 5 to 9% and 6 to 10%, 10 to 15%, 15 to 20%, 20 to 25%, 25 to 30% and more than 30%. In some embodiments, the stabilizing agent(s) can comprise between 1 and 5% by weight of the liquid suspension comprising the bulking agent, e.g., about 2 to 4%, or about 3.1%. Accordingly, the amount of stabilizing agent(s) in the liquid composition (based on the weight of the liquid composition before removal of water or other liquid solvent but after addition of a bulking agent if present), is typically from about 0.1 to about 30 wt %, such as from about 1 to about 10 wt % such as from about 2 to about 5 wt % e.g. from about 3 to about 4 wt % such as about 3.1 wt %.

Alternatively, the amount of stabilizing agent(s) can be calculated as a percent of the non-solvent components following addition of a bulking agent. As a percent of the non-liquid components, the stabilizing agent(s) can be present in an amount ranging from less than 2% up to 20% or more. Within that range are included the following ranges in percent by weight of 2 to 4%, 3 to 5%, 4 to 7%, 5 to 8%, 5 to 10%, 6 to 8%, 6 to 9%, 6 to 10%, 7 to 11%, 7 to 12%, 8 to 12%, 8 to 13%, 9 to 13%, 9 to 14%, 10 to 15%, 11 to 16%, 12 to 17%, 13 to 18% 14 to 19% and 15 to 20%, and more than 20%. For example, in certain embodiments the stabilizing agent can comprise between 5 and 15% by weight of the non-solvent components, e.g., between 9 and 13%, or about 11.2%. Such amounts will also correspond to the amounts of the stabilizing agent in the dry composition. Accordingly, the amount of stabilizing agent(s) in the composition (based on the weight of non-solvent components after addition of a bulking agent if present), is typically from about 2 to about 20 wt %, such as from about 4 to about 17 wt % such as from about 8 to about 15 wt % e.g. from about 10 to about 12 wt % such as about 11 wt % e.g. about 11.2 wt %.

b. Combinations of Stabilizing Agents

As noted above, combinations of stabilizing agents may be employed to assist in stabilizing the crystals in a liquid composition comprising oltipraz crystals as described in this Section A and/or assist in dispersing the crystals from a dry composition comprising oltipraz crystals as described in this Section A. For example, as noted above, in certain embodiments. nonionic surfactants may be combined with a cationic polymer or an anionic polymer. In other embodiments, an ionic surfactant (anionic or cationic) is combined with a neutral polymer. Other embodiments can combine a neutral polymer and nonionic surfactant.

Some exemplar combinations include, with or without an anti-foaming agent such as simethicone, (i) Eudragit RL in combination with Tween 80, Pluronic F-68 and/or sodium lauryl sulfate, (ii) Carbopol 974P NF RL in combination with Tween 80, Pluronic F-68 and/or sodium lauryl sulfate, (iii) PVP (K15 or K30) RL in combination with Tween 80, Pluronic F-68 and/or sodium lauryl sulfate. (iv) HPC EF RL in combination with Tween 80, Pluronic F-68 and/or sodium lauryl sulfate, and (v) HPMC E3 RL in combination with Tween 80, Pluronic F-68 and/or sodium lauryl sulfate, Some examples of such combinations are illustrated in Table 1 below.

TABLE 1

| Components | | Formulation Composition (mg/mL) | | | | | |
|---|---|---|---|---|---|---|---|
| Function | Name | 1 | 2 | 3 | 4 | 5 | 6 |
| API | Oltipraz | 50 | 50 | 50 | 50 | 50 | 50 |
| Stabilizing | Eudragit RL | 25 | | | | | |
| Agent | Carbopol 974P | | 5* | | | | |
| (Polymer) | NF | | | | | | |
| | PVP (K15 or | | | 25 | | | |
| | K-30) | | | | | | |
| | HPC EF | | | | 25 | | 25 |
| | HPMC E3 | | | | | 25 | |
| Stabilizing | Tween 80 | 12.5 | | | 12.5 | | |
| Agent | Pluronic F68 | | 12.5 | 12.5 | | 12.5 | |
| (Surfactant) | SLS | | | | | | 12.5 |
| Anti-foam | Simethicone | 0.5 | 0.5 | 0.5 | 0.5 | 0.5 | 0.5 |

*Added after milling

Among the various combinations, Eudragit RL in combination with Tween 80 or HPC EF in combination with Tween 80 have been found to provide acceptable results and typically to be particularly beneficial in terms of forming and keeping small crystals stable for a period of time. As discussed below, other combinations of the foregoing polymers and surfactants may be suitable depending on the particular composition and method of administration. The amounts of the individual components in such combinations are as set forth above for the individual components.

c. Other Surface-Active Agents

As noted above, surface active agents, including those listed above, may be added to the liquid compositions described in this Section A for purposes other than stabilizing oltipraz crystals, e.g., to aid in the dispersion of crystals upon resuspension with water and/or other liquid, or to serve other purposes beyond stabilizing the crystals, e.g., emulsifiers and anti-foam agents. For example, such ingredients can be added for the purpose of improving processes and/or compositions such as the processes for making the crystals or the properties of the composition comprising crystals.

In certain embodiments, e.g., an emulsifier may be added. Suitable emulsifiers include, but are not limited to, glycine soja protein, sodium lauroyl lactylate, polyglyceryl-4 diisostearate-polyhydroxystearate-sebacate, behentrimonium methosulfate-cetearyl alcohol, non-ionic emulsifiers like emulsifying wax, polyoxyethylene oleyl ether, PEG-40 stearate, carbomer, cetostearyl alcohol (cetearyl alcohol), ceteareth-12, ceteareth-20, ceteareth-25, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate. PEG-75 stearate, PEG-100 stearate, and PEG-100 stearate, ceramide 2, ceramide 3, stearic acid, cholesterol, laureth-12, steareth-2, and steareth-20, or combinations/mixtures thereof, as well as cationic emulsifiers like stearamidopropyl dimethylamine and behentrimonium methosulfate, or combinations/mixtures thereof.

In certain embodiments, an anti-foam agent may be added. Anti-foam agents may be used to reduce the formation of foam. e.g., in the process of making the crystals. Anti-foam agents that may be used include, but are not limited to, oil-based anti-foam agents [e.g., a hydrophobic silica or a wax (e.g., paraffin, ester waxes, fatty alcohol waxes, ethylene bis(stearamide)) in mineral or vegetable oil], powder defoamers, water-based defoamers (e.g., long chain fatty alcohols, fatty acid soaps, or esters in a white oil or vegetable oil), silicone-based defoamers [hydrophobic silica in silicone oil], polyethylene glycol- or polypropylene glycol-based defoamers, or alkyl polyacrylates. In certain preferred embodiments, the anti-foam agent is a silicone-based anti-foam agent. In certain embodiments, the anti-foam agent is poly(dimethylsiloxane), or silicon dioxide (simethicone).

Depending on the amount of water and/or other liquid used in the milling process, prior to the addition of a bulking agent, such additional surface-active ingredient(s) can be present in cumulative amounts ranging from less than 1 to more than 10% by weight of the liquid suspension. Within that range are included the following ranges in percent by weight, 0.1 to 1%, 1 to 3%, 1 to 4%, 1 to 5%, 2 to 5%, 2 to 6%, 3 to 6%, 3 to 7%, 4 to 7%, 4 to 8%, 5 to 8%, 5 to 9%, and 6 to 10%, and greater than 10%. For example, an anti-foam agent can be in an amount from about 0.01% to about 2% by weight of the liquid composition comprising the crystals, e.g. from about 0.01% to about 2%, from about 0.05% to about 1.5%, from about 0.1% to about 1%, from about 0.3% to about 0.9%, or from about 0.4% to about 0.8% by weight of the crystal. Typically, an anti-foam agent can be present in an amount from about 0.01% to about 2% by weight of the solid components (excluding bulking agents if present) in the liquid composition comprising the crystals, e.g. from about 0.01% to about 2%, such as from about 0.05% to about 1.5%, e.g. from about 0.1% to about 1%, such as from about 0.3% to about 0.9%. e.g. from about 0.4% to about 0.8% by weight of the crystal.

For example, a composition comprising oltipraz crystals of this Section A may typically comprise a combination of solubilizing agents selected from (i) one or more of acrylate- and alkenyl ether-based co-polymers, polyvinylpyrrolidone, hydroxypropylcellulose, hydroxypropyl methylcellulose, a copovidone such as PVP-VA64, and a polymethacrylate-based copolymer such as EUDRAGIT® RL; and (ii) one or more of sodium lauryl sulfate, a poloxamer such as Pluronic F-68 and polysorbate 80. Another surface-active ingredient may also be present such as an emulsifiers and/or an anti-foam agent. More typically the composition may comprise (i) one or more of a polymethacrylate-based copolymer such as Eudragit RL, an acrylate- and alkenyl ether-based co-polymer such as Carbopol 974P NF; a polyvinylpyrrolidone such as PVP (K15 or K-30); a hydroxypropylcellulose such as HPC EF and a hydroxypropyl methylcellulose such as HPMC E3; (ii) one or more of sodium lauryl sulfate, a poloxamer such as Pluronic F-68 and polysorbate 80; and optionally (iii) an antifoam agent such as poly(dimethylsiloxane) or silicon dioxide (simethicone). Still more typically the composition may comprise (i) one or more of a copovidone such as PVP-VA64 and a polymethacrylate-based copolymer such as EUDRAGIT® RL; (ii) polysorbate 80 (Tween 80); and optionally (iii) simethicone. In such compositions, the amount of component (i) is typically from about 5 to about 40 wt %, preferably from about 20 to about 35 wt % such as from about 25 to about 30 wt % based on the weight of solid components (excluding bulking agents) in the composition. The amount of component (ii) is typically from about 10 to about 20 wt %, preferably from about 12 to about 18 wt % such as from about 14 to about 15 wt % based on the weight of solid components (excluding bulking agents) in the composition. If present, the amount of component (iii) is typically from about 0.1 to about 1 wt %, preferably from about 0.3 to about 0.8 wt % such as from about 0.5 to about 0.7 wt % based on the weight of solid components (excluding bulking agents) in the composition.

d. Other Components

The liquid compositions described in this Section A also can comprise liquids in addition to water. For example, the liquid may be an aqueous buffer solution. Pharmaceutically acceptable buffers include acetate (e.g., sodium acetate), sodium carbonate, citrate (e.g., sodium citrate), tartrate, glycylglycine, histidine, glycine, lysine, arginine, sodium dihydrogen phosphate, disodium hydrogen phosphate, sodium phosphate, and tris(hydroxymethyl)-aminomethane or mixtures thereof. Alternatively, the compositions described herein as aqueous compositions may be instead prepared in a liquid solvent other than one that contains water, e.g., a polar organic solvent, such as methanol and/or ethanol. If liquids other than water are used, then advantageously, the liquid is one in which oltipraz is not more than minimally soluble, e.g., not more than 0.35%, 0.1%, 0.05%, 0.01%, 0.005%, 0.001%, or 0.0008% by weight solvated oltipraz in solvent. Typically, therefore, if liquids other than water are used, then advantageously, the liquid is one in which oltipraz is not more than minimally soluble, e.g., the liquid does not support more than 0.35%, e.g., not more than 0.1%, such as not more than 0.05%, e.g. not more than 0.01%, such as not more than 0.005%, e.g. not more than 0.001% or 0.0008% by weight solvated oltipraz in solvent. Combinations of liquids also may be used, including combinations of water and other liquids such as one or more polar organic solvents. Hence, although it is contemplated that aqueous compositions comprising oltipraz crystals as described in this Section A can be used throughout this disclosure, it is also contemplated that the water component in any of such aqueous compositions could be replaced in whole or in part by a liquid other than water. Where a solvent other than or in addition to water is used, the percentages given above for the stabilizing agents and other ingredients typically remain the same or substantially the same.

Liquid compositions comprising oltipraz crystals as described in this Section A, e.g., aqueous or otherwise, may be useful for milling. Other liquid compositions comprising oltipraz crystals as described in this Section A may be useful for spray-drying or lyophilization-based methods of generating the crystals in a dry composition. The total concentration of ingredients in such liquid formulations may be represented by the percentage by weight of combined solids in the formulation, wherein the combined solids are the non-solvent components. e.g., the crystals and any additives such as a stabilizing agent, surfactant, and/or a bulking agent that remain once the solvent is removed. The appropriate level of solids in a liquid composition as described in this Section A can vary depending on the use of the composition. For example, the total solids in a composition that is undergoing wet milling may be higher or lower than the total solids in a composition that also comprises a bulking agent and is undergoing a process in which liquid is being removed. e.g., spray-drying or lyophilization. In certain embodiments, for example including compositions for milling and/or spray-drying or lyophilization, the concentration of the solids in the liquid described herein can be about 5% to about 35% or more by weight, including ranges of from 5 to 10%, 10 to 15%, 10% to 20%, 15 to 20%, 15 to 25%, 20 to 25%, 20 to 30%, 25 to 30%, 25 to 35%, or more than 35%. In some embodiments, the total solids can be about 12% to about 18% by weight, about 13% to about 18% by weight, about 14% to about 17% by weight, or about 16% by weight of the formulation. Typically, therefore, the total solids can be from about 12% to about 18% by weight, e.g. from about 13% to about 18% by weight, such as from about 14% to about 17% by weight, e.g. about 16% by weight of the formulation. Typically, in some liquid compositions for milling, the total solids can be from about 20 to 30% by weight. e.g., from about 22 to about 27% by weight, such as about 25% by weight. In some embodiments. e.g., in some liquid compositions for milling, the total solids can be from about 20 to 30% by weight, e.g., about 22 to 27% by weight, about 25% by weight. In some embodiments, e.g., spray-drying compositions, the total solids can be from about 25 to 30% by weight. e.g., about 28% by weight. Using the guidance provided herein, one of ordinary skill will be able to determine an acceptable level of solids for compositions described in this Section A.

e. Crystal Sizes and Distribution, Such as Crystal Sizes and Distribution in Liquid Suspensions

Due to the inherent variability of the wet milling or other crystal-forming process such as antisolvent precipitation described in this Section A, the individual crystals of oltipraz formed from such processes will typically vary in size, and thus a quantity of oltipraz crystals produced by such processes can typically be characterized by a distribution of crystals of varying sizes. When in an aqueous suspension, the quantity of crystals described herein generally will have a MHD of between 30 and 2000 nm. Generally speaking, larger crystals will tend to settle faster in aqueous compositions, and so quantities of smaller crystals, e.g., those having an MHD from 30 to 100 nm, or 100 to 600 nm, including from 40 to 80 nm, 40 to 60 nm, or from 150 to 450 nm, 400 to 700 nm, 400 to 600 nm, and 450 to 550 nm, often provide an advantage in terms of better suspension characteristics over time for an aqueous suspension of the crystals, e.g., the crystals will remain substantially completely suspended longer. Generally speaking, production of crystals by wet milling will have an MHD above 100 nm, although MHD values below 100 nm may be obtained with longer milling times and or different milling parameters. Methods such as antisolvent precipitation can produce crystal compositions having MHD values in ranges below 100 nm. e.g., 30-100 nm, 40-80 nm and 40-60 nm. Within the MHD range of 30 to 2000 nm are MHD ranges of from 30 to 100 nm, 40 to 80 nm, 40 to 60 nm, 100 to 250 mu, 100 to 1200 n, 150 to 450 nm, 150 to 600 nm, 200 to 500 nm, 200 to 520, 200 to 600 nm, 300 to 600 nm, 300 to 700 nm, 300 to 800 nm, 400 to 600 nm, 400 to 700 nm, 800 nm, 500 to 750 nm, 750 to 1000 nm, 1000 to 1500 nm, and from 1500 to 2000 nm. Accordingly, the oltipraz crystals of the compositions described in this Section A typically have an intensity averaged (Z-average) MHD of from 30 to 1200 nm, such as from 100 to 600 nm, e.g. from 150 to 450 nm, 400 to 700 nm, 400 to 600 nm or 450 to 550 nm, e.g., from about 300 to 400 nm such as around 350 to 390 nm or from 400 to 600 nm such as around 500 nm, as measured by Dynamic Light Scattering.

It also is noted that the MHD measurements discussed herein also may reflect the presence of any additional ingredients such as the stabilizing agent(s) to the extent that they are present in the composition with the crystals. As used herein, however, MHD measurements obtained for complete aqueous dispersions comprising oltipraz crystals and one or more stabilizing agents, surfactants or other ingredients in the aqueous dispersion are deemed to be MHD measurements of the crystals themselves.

MHD can be determined by DLS using an appropriate instrument, e.g., a Malvern Zetasizer Nano-ZSP, using routine methods know to those skilled in the art. For example, the crystals as described in this Section A can be put into an aqueous suspension with deionized water to a concentration of 0.01-0.1 mg (based on the weight of oltipraz) per mL prior to analysis. The result will be a transparent orange-red suspension. A backscatter (173°) detector can be used. The temperature should be set to 25° C. and samples equilibrated for 90 seconds prior to analysis. The duration, number of runs, attenuator setting, and focal position can be set automatically by the software. MHD values can be recorded with attenuator settings of 4-6 with mean count rates of 180-500 keps.

All calculations of crystal size discussed herein can be performed in the Malvern Zetasizer software. As noted above, average crystal sizes discussed herein are intensity-averaged mean hydrodynamic radius (Z-average), The size is calculated from the mean decay time of the autocorrelation function and the Stokes-Einstein equation. The viscosity of water at 25° C. (0.8872 cP) was used. In cases where a crystal size distribution is given, the Malvern General Purpose (normal resolution) method is used, which uses non-negative least squares (NNLS) fitting of the decay curves. The functioning of the Malvern Zetasizer can be periodically checked using 100 nm polystyrene beads calibration standard. The relaxation time in the DIS experiment is between 600 and 1500 microseconds with the preferred relaxation time between 500 and 1300 microseconds.

The size calculation for the crystal sizes reported herein is based on a cumulant method using the equation: Γq2=D=kBT/3πηd where D is the diffusion coefficient calculated from the measured decay rate (Γ), kBT/3πηd is the Stokes-Einstein equation, d is the particle diameter, and q is the scattering wave vector which is dependent on the specific instrument method parameters as listed above. The magnitude of the scattering wave vector is calculated according to the equation q=4 Pi (refractive index of solvent) Sin(theta/2)/wavelength. The expected delay time will change if a different instrument uses a different value of q. For calculations used herein, theta=173 deg, a refractive index of 1.333 for water is used, a laser wavelength of 633 nm yields a value for q=0.0264 nm\hat{ }(−1).

As discussed above, the inherent variability of the wet milling process means that the size of individual crystals in any given quantity of crystals will vary and thus a quantity of crystals prepared according to this disclosure can be characterized by a distribution of crystals of varying sizes. One measure of the distribution of sizes is the polydispersity index (PdI) of the crystals in the quantity. The formula for determining PdI is:

PdI=(σ/d)²

where σ is the standard deviation and d is the mean hydrodynamic diameter (Z-average) is less than 0.80, wherein PdI=(σ/d)², wherein σ is the standard deviation and d is the mean hydrodynamic diameter (Z-average). Lower values of PdI indicate a more uniform distribution of crystals in a given quantity of crystals. Typically, oltipraz crystals or quantities of such crystals in accordance with this Section A can have a PdI of less than 1, usually less than 0.8, often less than 0.6; for example between 0.10 and 0.60, e.g. between 0.10 and 0.45, such as between 0.1 and 0.35 e.g. 0.1 and 0.25. Certain embodiments of quantities of crystals in accordance with this Section A can have a PdI of less than 1, less than 0.8, less than 0.6. e.g., between 0.10 and 0.60, and between 0.10 and 0.45, 0.1 and 0.35 and 0.1 and 0.25.

Typically, therefore, oltipraz crystals prepared as discussed in this Section A will have an intensity averaged (Z-average) MHD (as measured by Dynamic Light Scattering) of from 30 to 1200 nm, wherein the PdI of the crystals is from 0.1 to 0.6. More typically such oltipraz crystals will have an intensity averaged (Z-average) MHD of from about 100 to about 600 nm, wherein the PdI of the crystals is from 0.1 to 0.45. Still more typically such oltipraz crystals having an intensity averaged (Z-average) MHD of from about 150 to about 450 nm, 400 to 700 nm, 400 to 600 nm, or 450 to 550 nm, wherein the PdI of the crystals is from 0.1 to 0.6, or from 0.1 to 0.35.

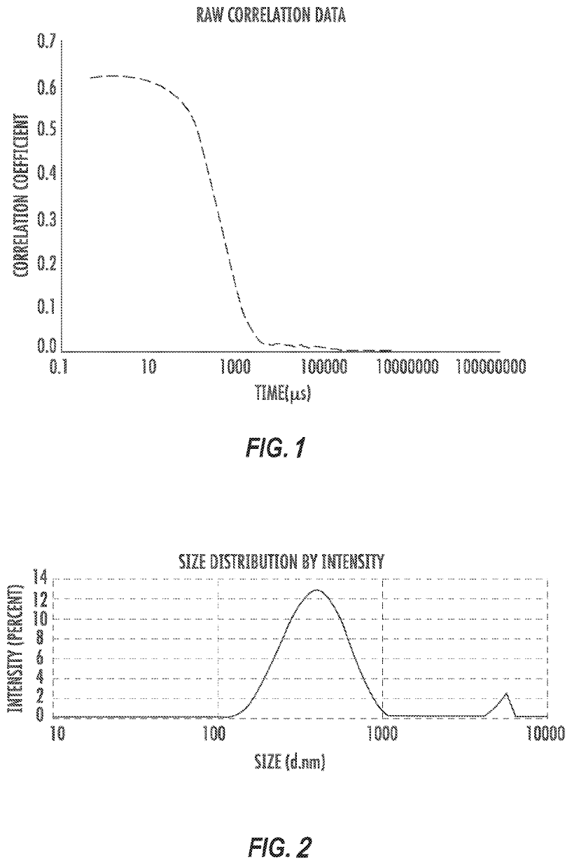

FIGS. 1 and 2 illustrate a correlogram and intensity size distribution for a DLS analysis of a sample of suspended crystals (prepared as described in this Section A) at 25° C. The relaxation time is 1180 microseconds. MHD is 403 nm, and the PdI is 0.364. It is noted that the x axis for both plots is logarithmic. The MHD and PdI are both calculated by the instrument based on the data obtained and are not determined visually from the figures.

2. Dry Compositions Comprising Oltipraz Crystals

As discussed above, the liquid compositions comprising crystals in suspension described in this Section A can be admixed with a bulking agent and then spray dried, lyophilized or otherwise processed to remove the water and/or other liquid solvent to form a dry composition. The resulting dry composition can comprise particles that largely comprise the bulking agent and thus can be much larger than the oltipraz crystals. For example, particles up to 200 microns (200,000 nm) or larger may be obtained. If desired, the size of the particles obtained from processes such as spray drying may be measured by scanning electron microscopy, laser diffraction or light microscopy. Dry compositions of crystals prepared according to this Section A generally will be in the form of an orange-red powder and can be prepared with no discolorations or large particles or chinks visible.

a. Bulking Agents

The presence of a bulking agent reduces the likelihood of crystal-crystal surface contact in a dry composition such as a spray-dried or lyophilized powder, as direct contact can make the crystals harder to re-suspend where the ultimate use of the composition is resuspension in a liquid composition. Bulking agents that are generally very soluble in water may be able to release the crystals as individual crystals upon resuspension. Accordingly, bulking agents that are very soluble in water are typically used in compositions described in this Section A. Those skilled in the art are capable of choosing appropriate bulking agents based on the particular composition and intended route of delivery. Furthermore, because the bulking agent can be such a large fraction of the overall dry composition product of crystals, its properties may affect the rate of resuspension in water as well as potentially influence the taste of the composition if administered orally, possibly significantly.

One factor that can be evaluated to determine if a particular bulking agent is appropriate for a particular embodiment includes whether the bulking agent does not alter the initial size of the oltipraz crystals in suspension prior to removal of water, e.g., through spray dying or lyophilization. Where the intended use of the dry composition is resuspension with water or other liquid to make a liquid composition for oral or other form of administration, then advantageously, a bulking agent is typically chosen that (i) does not yield large particles of precipitate upon resuspension with water. (ii) does not yield a dry powder that dissolves too slowly upon mixing with water, and (iii) yields a dry powder that is relatively stable to handling and storage, e.g., is not hygroscopic such that handling of the dry composition becomes difficult. Surface active agents may be added to the formulation, either in the liquid compositions discussed above or to the dry composition in order to enhance such properties in the dry composition. Such properties may be less important, however, if the dry composition is to be formulated into a pill, tablet, capsule, gel capsule or the like for oral administration. Where the intended use of the dry composition is oral administration such as in a pill, tablet or capsule, then the bulking agent also should be evaluated on its ability to provide the desired pharmacological profile following administration. If the smaller oltipraz crystalline drug particles coated with a stabilizing agent are adsorbed onto the larger particles of the bulking agent during blending or granulation, such as roller compaction, fluid bed, or high shear, then a water soluble bulking agent such as mannitol, or insoluble agent such as microcrystalline cellulose, may act as a carrier for those particles and aid the rate of dissolution from a capsule or a tablet.

As noted above, in principle, a bulking agent also can act as a stabilizing agent. Examples of bulking agents include, but are by no means limited to, the group consisting of polyvinylpyrrolidones (e.g., PVP K30 and PVP-VA64), cellulosic polymers such as HPC, HPMC, HPMC E3. Trehalose, and Dextrans such as Dextran 10 or Dextran 40. Examples of bulking agents such as PVP-VA64 and HPC EF that provide acceptable results for certain embodiments of this disclosure are provided herein. Most typically the bulking agent is PVP-VA64. Sometimes it is preferable that the bulking agent is not Dextran 40. As noted above, appropriate bulking agents or combinations of bulking agents can be determined for a particular composition and route of delivery. Factors such as the intended route of administration of the crystals (e.g., whether the crystals are to be administered in a dry form such as a pill or capsule or resuspended with a liquid such as water), all may be considered in determining one or more acceptable bulking agents for a particular embodiment. Other factors such as the size and amounts of crystals, type and quantity of stabilizing agent used (if any), the surfactants and amounts thereof (if any) that are employed, the amount of bulking agent to be used, the total solids in the liquid composition, the liquids in the composition and any resuspension, and the process for removing water (and/or other liquid), also may be taken into account in determining acceptable bulking agents or combinations of bulking agents.

In some circumstances use of Dextran 10 may provide a dry composition that provides particle sizes that are too large upon resuspension in water. In other embodiments, HPMC may provide a composition that dissolves more slowly than desired upon resuspension with water. In some embodiments, Trehalose can provide a composition that is more hygroscopic than desired for routine handling. Special packaging or the addition of desiccant may be used to maintain the low water content of such hygroscopic pharmaceutical compositions during stability on the shelf. Accordingly, it is sometimes preferable that the bulking agent is not dextran 10 and/or is not HPMC and/or is not trehalose. In different embodiments however, e.g., with different stabilizing agents, surfactants, or for a different intended route of administration, such bulking agents can provide acceptable compositions.

Within the aqueous or liquid compositions described in this Section A, depending on the amount of liquid used, the bulking agent(s) can comprise from about 1 to 40% by weight or more of the composition. Within such ranges are, e.g., 1 to 5%, 5 to 10%, 10 to 15%, 10 to 20%, 15 to 20%, 15 to 25%, 20 to 25%, 20 to 30%, 25 to 30%, 25 to 35%, 30 to 35%, 30 to 40%. Depending on the method chosen for removing water, the total solids in the composition may have to be maintained below a certain level to facilitate processing to a dry composition, e.g., in certain embodiments, below 30%, or about 28%, and thus the amount of bulking agent(s) used may be limited by such considerations. In certain embodiments, therefore, the bulking agent can comprise between 15% and 25%, e.g., about 20 or 21%. Accordingly, the bulking agent(s) typically comprise from about 1 to about 40 wt % of the liquid composition, such as from about 10 to about 30 wt % e.g. from about 15 to about 25 wt % such as from about 20 to about 21 wt %.

Alternatively, as with the other ingredients, the amount of bulking agent can be calculated as a percent of the solids, i.e., the non-solvent components. As a percent of the solids, the bulking agent(s) can be present in amounts by weight ranging from less than 40% up to 98% or more, e.g., 40 to 50%, 50 to 60%, 55 to 65%, 60 to 70%, 60 to 75%, 60 to 80%, 65 to 75%, 65 to 80%, 70 to 80%, 75 to 85%, 75 to 90%, 80 to 90%, 80 to 95%, 85 to 95%, 90 to 98%, and greater than 98% by weight. In certain embodiments, the bulking agent(s) can comprise between 65 and 80% by weight of the total solids. e.g., between about 70 and 78%, e.g., about 74% by weight of the total solids. Accordingly, the bulking agent(s) typically comprise from about 40 to about 90 wt % of the non-solvent (i.e., solid) composition, such as from about 65 to about 80 wt % e.g. from about 70 about 78 wt % such as from about 73 to about 75 wt %. Such amounts will also correspond to the amounts of the bulking agent(s) in the dry composition.

The compositions of crystals of oltipraz described in this Section A thus can have a MHD of from about 30 to about 2000 nm as measured by dynamic light scattering, wherein the crystals typically have a solubility in water at 20° C. of from about 3.5 to about 8 μg/ml. More typically such compositions of crystals of oltipraz have a MHD of from about 100 to about 800 nm as measured by dynamic light scattering, wherein the crystals typically have a solubility in water at 20° C. of from about 4.5 to about 7 μg/ml. Still more typically such compositions of crystals of oltipraz have a MHD of from 150 to about 450 nm, 400 to 700 nm, 400 to 600 nm, or 450 to 550 nm, as measured by dynamic light scattering, wherein the crystals typically have a solubility in water at 20° C. of from about 5 to about 6.5 μg/ml

Thus, for example, a liquid composition is provided according to this Section A, wherein:
the composition comprises between about 1 to about 40 wt % of oltipraz crystals, based on the weight of the liquid composition;
the non-solvent components in the composition typically comprise from about 1 to about 70 wt % oltipraz crystals; and
the composition comprises (i) from about 5 to about 40 wt % (based on the weight of solid components in the composition) of one or more of acrylate- and alkenyl ether-based co-polymers, polyvinylpyrrolidone, hydroxypropylcellulose, hydroxypropyl methylcellulose, a copovidone such as PVP-VA64, and a polymethacrylate-based copolymer such as EUDRAGIT® RL; and/or (ii) from about 10 to about 20 wt % % (based on the weight of solid components in the composition) of one or more of sodium lauryl sulfate, a poloxamer such as Pluronic F-68 and polysorbate 80.

Thus, for example, another liquid composition is provided according to this Section A, wherein:
the composition comprises between about 4 to about 15 wt % of oltipraz crystals, based on the weight of the liquid composition;
the non-solvent components in the composition typically comprise from about 50 to about 60 wt % oltipraz crystals;
the composition comprises (i) from about 20 to about 35 wt % (based on the weight of solid components in the composition) of one or more of a polymethacrylate-based copolymer such as Eudragit RL, an acrylate- and alkenyl ether-based co-polymer such as Carbopol 974P NF; a polyvinylpyrrolidone such as PVP (K15 or K-30); a hydroxypropylcellulose such as HPC EF and a hydroxypropyl methylcellulose such as HPMC E3; and/or (ii) from about 12 to about 18 wt % % (based on the weight of solid components in the composition) of one or more of sodium lauryl sulfate, a poloxamer such as Pluronic F-68 and polysorbate 80;
the liquid solvent is water or an aqueous buffer solution; and
the composition optionally comprises from 0.1 to 1 wt % of poly(dimethylsiloxane) or silicon dioxide (simethicone) based on the non-solvent components in the composition.

Thus, for example, another liquid composition is provided according to this Section A, wherein:
the composition comprises between about 7 to about 10 wt % of oltipraz crystals, based on the weight of the liquid composition;
the non-solvent components in the composition typically comprise from about 55 to about 58 wt % oltipraz crystals;
the composition comprises (i) from about 25 to about 30 wt % (based on the weight of solid components in the composition) of one or more of a copovidone such as PVP-VA64 and a polymethacrylate-based copolymer such as EUDRAGIT® RL; and/or
(ii) from about 14 to about 15 wt % % (based on the weight of solid components in the composition) of polysorbate 80 (Tween 80);
the liquid solvent is water; and
the composition optionally comprises 0.1 to 1 wt % simethicone based on the non-solvent components in the composition.

The liquid composition comprising oltipraz crystals but not comprising a bulking agent is typically suitable for milling.

For example, another liquid composition is provided according to this Section A, wherein:
The concentration of oltipraz crystals in the liquid is from about 0.1 to about 10 wt % based on the weight of the liquid composition;
the non-solvent components in the composition typically comprise from about 0.5 to about 25 wt % oltipraz crystals;
the composition comprises (i) from about 5 to about 40 wt % (based on the weight of solid components excluding bulking agents in the composition) of one or more of acrylate- and alkenyl ether-based co-polymers, polyvinylpyrrolidone, hydroxypropylcellulose, hydroxypropyl methylcellulose, a copovidone such as PVP-VA64, and a polymethacrylate-based copolymer such as EUDRAGIT® RL; and/or (ii) from about 10 to about 20 wt % % (based on the weight of solid components excluding bulking agents in the composition) of one or more of sodium lauryl sulfate, a poloxamer such as Pluronic F-68 and polysorbate 80; and
the composition comprises from about 1 to about 40 wt % (based on the overall weight of the composition) of a bulking agent selected from polyvinylpyrrolidones (e.g., PVP K30 and PVP-VA64), cellulosic polymers such as HPC. HPMC, HPMC E3, Trehalose. Dextrans (such as Dextran 10 or Dextran 40). PVP-VA64 and HPC EF.

For example, another liquid composition is provided according to this Section A, wherein:
The concentration of oltipraz crystals in the liquid is from about 1 to about 6 wt % based on the weight of the liquid composition;
the non-solvent components in the composition typically comprise from about 5 to about 20 wt % oltipraz crystals;
the composition comprises (i) from about 20 to about 35 wt % (based on the weight of solid components excluding bulking agents in the composition) of one or more of a polymethacrylate-based copolymer such as Eudragit RL, an acrylate- and alkenyl ether-based co-polymer such as Carbopol 974P NF; a polyvinylpyrrolidone such as PVP (K15 or K-30); a hydroxypropylcellulose such as HPC EF and a hydroxypropyl methylcellulose such as HPMC E3; and/or (ii) from about 12 to about 18 wt % % (based on the weight of solid components excluding bulking agents in the composition) of one or more of sodium lauryl sulfate, a poloxamer such as Pluronic F-68 and polysorbate 80;
the composition comprises from about 10 to about 30 wt % (based on the overall weight of the composition) of a bulking agent selected from PVP-VA64 and HPC EF;
the liquid solvent is water or an aqueous buffer solution; and
the composition optionally comprises from 0.1 to 1 wt % of poly(dimethylsiloxane) or silicon dioxide (simethicone) based on the non-solvent components (excluding the bulking agent) in the composition.

For example, another liquid composition is provided according to this Section A, wherein:
The concentration of oltipraz crystals in the liquid is from about 2 to about 5 wt % based on the weight of the liquid composition;
the non-solvent components in the composition typically comprise from about 10 to about 18 wt % oltipraz crystals;
the composition comprises (i) from about 25 to about 30 wt % (based on the weight of solid components excluding bulking agents in the composition) of one or more of a copovidone such as PVP-VA64 and a polymethacrylate-based copolymer such as EUDRAGIT® RL; and/or (ii) from about 14 to about 15 wt % % (based on the weight of solid components excluding bulking agents in the composition) of polysorbate 80 (Tween 80);
the composition comprises from about 15 to about 25 wt % (based on the overall weight of the composition) of a bulking agent which is PVP-VA64;
the liquid solvent is water; and
the composition optionally comprises 0.1 to 1 wt % simethicone based on the non-solvent components (excluding the bulking agent) in the composition.

The liquid composition comprising oltipraz crystals and a bulking agent according to this Section A is typically suitable for drying e.g. spray-drying.

Thus, for example, this Section A provides dry compositions comprising oltipraz crystals and a bulking agent, wherein:
The percentage of oltipraz in the composition (i.e. the drug loading) is from about 12 to about 20 wt %;
the composition comprises (i) from about 5 to about 40 wt % (based on the weight of solid components excluding bulking agents in the composition) of one or more of acrylate- and alkenyl ether-based co-polymers, polyvinylpyrrolidone, hydroxypropylcellulose, hydroxypropyl methylcellulose, a copovidone such as PVP-VA64, and a polymethacrylate-based copolymer such as EUDRAGIT® RL; and/or (ii) from about 10 to about 20 wt % % (based on the weight of solid components excluding bulking agents in the composition) of one or more of sodium lauryl sulfate, a poloxamer such as Pluronic F-68 and polysorbate 80; and
the composition comprises from about 40 to about 90 wt % (based on the overall weight of the composition) of a bulking agent selected from polyvinylpyrrolidones (e.g., PVP K30 and PVP-VA64), cellulosic polymers such as HPC. HPMC. HPMC E3. Trehalose, Dextrans (such as Dextran 10 or Dextran 40). PVP-VA64 and HPC EF.

For example, another dry composition comprising oltipraz crystals and a bulking agent is provided by this Section A, wherein:
The percentage of oltipraz in the composition (i.e. the drug loading) is from about 14 to about 18 wt %;
the composition comprises (i) from about 20 to about 35 wt % (based on the weight of solid components excluding bulking agents in the composition) of one or more of a polymethacrylate-based copolymer such as Eudragit RL, an acrylate- and alkenyl ether-based co-polymer such as Carbopol 974P NF; a polyvinylpyrrolidone such as PVP (K15 or K-30); a hydroxypropylcellulose such as HPC EF and a hydroxypropyl methylcellulose such as HPMC E3; and/or (ii) from about 12 to about 18 wt % % (based on the weight of solid components excluding bulking agents in the composition) of one or more of sodium lauryl sulfate, a poloxamer such as Pluronic F-68 and polysorbate 80;
the composition comprises from about 65 to about 80 wt % (based on the overall weight of the composition) of a bulking agent selected from PVP-VA64 and HPC EF; and
the composition optionally comprises from 0.1 to 1 wt % of poly(dimethylsiloxane) or silicon dioxide (simethicone) based on the weight of solid components excluding bulking agents in the composition

For example, another dry composition comprising oltipraz crystals and a bulking agent is provided by this Section A, wherein:
The percentage of oltipraz in the composition (i.e. the drug loading) is from about 15 to about 17 wt %;
the composition comprises (i) from about 25 to about 30 wt % (based on the weight of solid components excluding bulking agents in the composition) of one or more of a copovidone such as PVP-VA64 and a polymethacrylate-based copolymer such as EUDRAGIT® RL; and/or (ii) from about 14 to about 15 wt % % (based on the weight of solid components excluding bulking agents in the composition) of polysorbate 80 (Tween 80); and
the composition comprises from about 70 to about 78 wt % (based on the overall weight of the composition) of a bulking agent which is PVP-VA64; and the composition optionally comprises 0.1 to 1 wt % simethicone based on the weight of solid components excluding bulking agents in the composition.

The dry compositions described above can be suspended in liquid to form a liquid suspension; typically the weight ratio of the solid:liquid is from about 1:10 to 1:200 such as from about 1:20 to 1:150 e.g. 1:30 to 1:100.

The oltipraz crystals in the liquid compositions described in this Section A typically retain a MHD of from 30 to 1200 nm for at least 1 hour; more typically the oltipraz crystals retain a MHD of from 100 to 800 nm for at least 6 hours; still more typically the oltipraz crystals retain a MHD of from 150 to 450 nm, 400 to 700 nm, 400 to 600 nm, or 450 to 550 nm for at least 24 hours.

The oltipraz crystals in the solid compositions described in this Section A typically have a solubility in water at 20° C. of from about 3.5 to about 8 μg/ml, more typically from about 4.5 to about 7 μg/ml, still more typically from about 5 to about 6.5 μg/ml.

3. Methods of Making Compositions Comprising Oltipraz Crystals

Methods of making the oltipraz crystal compositions described in this Section A typically provide advantages due to their scalability. The methods described herein can be used for large, commercial-scale production (e.g., kilogram quantities), of compositions comprising the oltipraz crystals. Moreover, certain embodiments of the methods described herein can provide compositions comprising crystals of oltipraz with a bulking agent made from aqueous composition using water-removal methods such as spray-drying or lyophilization. Hence, such embodiments do not generate a large amount of organic solvent waste.

a. Wet Milling

Oltipraz may be synthesized or may be obtained from commercial vendors, e.g. Sigma-Aldrich® and Santa Cruz Biotechnology®. Inc. Methods for synthesizing oltipraz (4-Methyl-5-(2-pyrazinyl)-1,2-dithiole-3-thione) have been described in the art. (See e.g. U.S. Pat. No. 4,110,450 and Framroze PCT/IN2016/050197).

Wet milling of the oltipraz can be carried out by known processes. For example, the oltipraz can first be suspended in water to form an aqueous composition. A different liquid may be used in addition to, or in place of water. The oltipraz suspension can be milled in a temperature-controlled grinding chamber (such as a Dyno-mill, model KDL) using a grinding media such as 0.5 mm yttrium-stabilized zirconium oxide spheres. The total grinding time is chosen so as to provide a target MHD as measured by DLS, as described above. The time for grinding varies with the type of mill, and whether it is recirculating. While a Dyno-mill may be suitable for smaller batches, other larger mills, such as Netzsch mills, can adapt the process to much larger scales of batches of crystals with the same target MHD. As discussed above, one or more stabilizing agents and/or surfactants may be added to the wet-milling composition. Where at least one stabilizing agent is provided, the crystals may be stable in the liquid composition for a period of time. That is, the MHD of the crystals can remain within a target range for a period of time, e.g., at least 1 hour, 6 hours, 12 hours, 24 hours, 48 hours and 72 hours. The weight percent of oltipraz in the liquid milling composition can vary from 1% up to 20% percent or more (excluding the weight of the milling media). Within such range are the following sub-ranges. i.e., 1 to 5%, 5 to 10%, 5 to 15%, 10 to 15%, 10 to 20%, and more than 20%. In certain embodiments, prior to the addition of bulking agent, the loading of oltipraz during milling is between about 5 and 10% by weight of the aqueous composition, or about 8.6%. In other embodiments, prior to the addition of bulking agent the loading of oltipraz and other non-aqueous components such as the stabilizing agent(s) during milling may be between 13 and 17%, e.g., about 15%, which represents a high loading of solids for wet milling.

During milling, the temperature can be less than 40° C. but above 2° C. to avoid the composition approaching the freezing point. Generally speaking, however, colder is better to minimize both chemical degradation (to avoid drug-degradant impurities) and to lower the solubility of the compound so the milled crystals do not grow due to a dissolution/recrystallization mechanism. Using such conditions can minimize drug-degradant impurities relative to 4-methyl-5-(pyrazin-2-yl)-3H-1,2-dithiole-3-thione in the aqueous composition to less than 1%. e.g. less than 0.5%, e.g. less than 0.1%, and minimize the drug-degradant impurities to less than 2% such as less than 1% or less than 0.5% relative to the 4-methyl-5-(pyrazin-2-yl)-3H-1,2-dithiole-3-thione in the aqueous suspension. Typically, such conditions can minimize drug-degradant impurities relative to 4-methyl-5-(pyrazin-2-yl)-3H-1,2-dithiole-3-thione in the aqueous composition to less than 1%, less than 0.5%, or less than 0.1%, and minimize the drug-degradant impurities to less than 2%, less than 1% or less than 0.5% relative to the 4-methyl-5-(pyrazin-2-yl)-3H-1,2-dithiole-3-thione in the aqueous suspension. In certain embodiments, the temperature may be maintained at about 10° C. The liquid compositions prepared from milling may be used to prepare additional compositions, e.g., pharmaceutical compositions. Alternatively, where a dry composition of the crystals is desired, the liquid compositions may be subjected to further processing as discussed below to effect removal of the water and/or other solvent liquid.

b. Other Crystal-Formation Processes

As noted above, oltipraz crystals may be made by methods other than milling. For example, crystals of oltipraz may be prepared by antisolvent precipitation, supercritical fluid precipitation or other known means of producing compositions comprising particles having an MHD in the size ranges described herein. Stabilizing agents may be added as in the wet milling process and removal of liquids may still be necessary.

c. Liquid Removal

Once the target MHD of the oltipraz crystals is reached, all or a portion of the suspension then may be mixed with one or more hulking agents as described above. The resulting mixture may be further diluted as desired to achieve the desired target solids content prior to further processing to remove the water and/or other liquid from the composition. The final suspension may be stirred prior to the step of removing the liquid.

Where the liquid of the composition is water, known processes such as spray drying or lyophilization may be used to remove the water from the composition. An exemplary spray-drying process is provided below in Example 1. The resulting composition then may be further processed as desired. The powder is preferably stable for a minimum period of time, e.g., at least one month, at least two months, at least three months, or at least six months, one year, two years, or more than two years. Stability of the powder may be measured at room temperature (e.g., 70° F. or 21° C.) or at a temperature below room temperature (e.g., 5° C.) or at a higher temperature and relative humidity, e.g., 40° C. and 75% RH. Stability of the powder may be measured according to a number of parameters, including purity, potency, or ability to re-suspend and remain substantially re-suspended in a liquid composition (see Example 4).

When milling and spray drying are employed in combination, the following parameters may need to be considered and adjusted to achieve acceptable or optimal results.

Throughput: This can be an important process consideration as it can dictate how high the solids loading will be during the wet milling step and liquid removal. That is, the higher the desired throughput, the higher the solids loading required during milling and spray-drying. A high-solids loading in the milling step is about 15%, although higher amounts such as about 20% may be achieved. Further, one can mill at a high-solids loading (e.g., 15 wt %) and not dilute the aqueous composition with water (i.e., avoid a washing step to recover more product) at any point. Then this high solids-loaded composition can be fed into spray drying and sprayed at a high-solids loading. e.g., about 28% solids. The desire to push throughput can be dictated by the fact that the spraying is done out of water where the high dew point of water relative to organic solvents at similar vapor composition limits the rate at which one can spray dry.

Nozzle and drying gas flow rate: In certain embodiments, spray drying such solutions at high throughput can be facilitated by using a two-fluid nozzle for atomization and adjusting the atomization gas flow rate to get the desired particle size distribution. By maintaining a sufficient drying gas flow rate, the process can be relatively insensitive to fluctuations in solution/suspension flow rate. If the atomization gas rate is too low, however, then particle size can become very sensitive to suspension flow fluctuations. Running in the more robust regime can be important because the highly viscous spray suspension can be difficult to run at the necessary flow rate without significant fluctuations.

Time: Total residence time of the oltipraz crystals in the grinding environment is a parameter for milling. For a given set of milling conditions, e.g., oltipraz loading, the wet-milling machinery and milling media used, milling temperature, and target particle size ae among the parameters that will dictate the total residence time for milling. Compositions of crystals having smaller MHD values typically will require longer milling times, and one of ordinary skill will be able to determine the milling time necessary for a desired MHD through routine experimentation.

Milling machinery and parameters: For a given target crystal size, one of ordinary skill can find a combination of wet-milling machinery and wet-milling media that can achieve the target crystal size. For example, a target range of MHD between 150 and 600 nm, e.g., 150 to 450 nm, can be achieved with either DynoMill or LabRAM milling machinery. For such MHD ranges, a combination of a rotor speed for the DynoMill of about 3000 rpm and 0.5 mm grinding spheres can be used. For LabRAM, acceleration of 50 g and using a combination of 0.2 mm and 0.6 mm grinding spheres can provide acceptable results. The two systems will require different times however.

As noted above, in other embodiments, the crystals may be made by precipitation, antisolvent precipitation, super critical liquid precipitation, fluid bed granulation, wet-impregnation, evaporation (e.g., rotary evaporation, vacuum drying) and other methods known to persons of ordinary skill in the art.

B. Active Pharmaceutical Ingredients that can Reduce Cellular Oxygen Consumption Rate (OCR-APIs)

As discussed above, the oltipraz, whether simply recrystallized (e.g., U.S. Pat. No. 4,110,450 and Framroze PCT/1N2016/050197), or further processed and formulated as described in Section A above, can be co-administered with a second API that can reduce the rate of cellular oxygen consumption and thereby enhance the prophylactic and/or therapeutic benefit of the oltipraz (OCR-APIs). Known OCR-APIs include meclizine, nimorazole, metformin, phenformin. antimycin A, pyrvinium, berberine, niclosamide, acriflavinium, sorafenib, emetine, plicamycin, suloctidil, pentamidine, amsacrine, irinotecan, itraconazole, mitomycin, hydroxyprogesterone, cyclosporine, fenofibrate, and analogues of ubiquinone such as atovaquone. See. e.g., Ashton et al.

As used in this disclosure, the term OCR-API is intended solely to denote compounds that qualify under the assay described below, which assay is the OCR primary screen described in Ashton et al. Such compounds thus have the potential to exert the pharmaceutical effect of reducing the cellular oxygen consumption rate. Such compounds can include compounds that have been approved for some pharmaceutical use in humans, as well as compounds that have not yet been approved for use in humans and/or which for one or more reasons (e.g., toxicity, patient tolerability and/or side effects), ultimately may not be approved for use in humans.

One of the OCR-APIs described above is atovaquone, which is the name for trans-2-[4-(4-chlorophenyl)cyclohexyl]-3-hydroxy-1,4-naphthalenedione. Atovaquone is a USFDA-approved drug that is marketed in a liquid form, or oral suspension, by GlaxoSmithkline LLC under the trade name Mepron. Atovaquone is available from Sigma as a powder (yellow in color), and like oltipraz, atovaquone has a very low solubility in water. Atovaquone is FDA approved for the prevention and treatment of Pneumocystis jirovecii pneumonia (PCP), but also is used to treat malaria, toxoplasmosis, and babesiosis (which can be caused by deer tick bites). Atovaquone also is available in tablet form, often together with Proguanil hydrochloride for treatment and prophylaxis of malaria and PCP. As discussed below, atovaquone has been found to enhance the protective/therapeutic effect of oltipraz for mucosal cells.

1. Assay for Determining an OCR-API

The following assay, which is the OCR primary screen described in Ashton et al., is the assay to determine if a compound qualifies for purposes of this disclosure as an OCR-APT.

To identify compounds that reduce the OCR, FaDu hypopharyngeal carcinoma cells are incubated with compounds at 2 or 10 mM for 24 h. The growth medium is replaced with an assay medium containing 5 mM galactose, 5 mM pyruvate and 4 mM glutamine. Growth in galactose promotes oxidative phosphorylation. The mitochondrial-specific OCR is determined by measuring basal OCR and subtracting the OCR measured following injection of 2 mM antimycin A to inhibit mitochondrial respiration. The OCR measurements are corrected for cell number using the relative Hoechst fluorescence of the cells fixed immediately after the assay. Compounds that cause a reduction in cell number of 66% or less compared with the DMSO control wells for each plate are included as OCR-APIs. Compounds that cause a reduction in cell number of more than 66% compared with the DMSO control wells for each plate are excluded as OCR-APIs. Further details of the assay for determining OCR are as follows.

FaDu cells are incubated with an API at 2 and 10 mM for 24 h in DMEM containing 5 mM glucose and 4 mM L-glutamine. The mitochondrial-specific OCR is determined by taking a basal OCR measurement in XF assay medium (Seahorse Biosciences) containing 5 mM galactose, 5 mM sodium pyruvate and 4 mM L-glutamine using an XF96 Analyzer (Seahorse Biosciences) and subtracting the OCR measured following injection of 2 mM antimycin A. The cells are then fixed in ice-cold methanol for 5 min, and incubated in 1×PBS, 4 mgml-1 Hoechst 33258 (Sigma) for 30 min before measuring fluorescence using a POLARstar Omega plate reader (BMG Labtech) to obtain the relative cell number per well. The mitochondrial-specific OCR is corrected using the relative cell numbers, and compounds that cause a reduction in cell number of 66% or less compared with the DMSO control wells for each plate are included as an OCR-API. Compounds that cause a reduction in cell number of more than 66% compared with the DMSO control wells for each plate are excluded as an OCR-API. Two repetitions of the screen are conducted. Ashton et al. should be consulted if any further details of the above OCR primary screen are required.

C. Pharmaceutical Compositions and Administration of Oltipraz and an OCR-API

The oltipraz (e.g., either recrystallized or formulated as described above in Section A) and/or other Nrf2 activator(s), either with or without one or more OCR-APIs such as atovaquone, can be administered at the same time in the same dry or liquid composition or in separate dosages that are administered substantially together or at different times. Alternatively, they can be administered separately, with the dosage of oltipraz and/or other Nrf2 activator preceding the dosage of OCR-API. e.g., by one hour or less, two hours or less, six hours or less, twelve hours or less, twenty-four hours or less, or by more than twenty-four hours. Alternatively, the dosage of OCR-API can precede the dosage of oltipraz and/or other Nrf2 activator(s), e.g., by one hour or less, two hours or less, six hours or less, twelve hours or less, twenty-four hours or less, or by more than twenty-four hours. For example, a patient can administer a daily dose (or daily doses) of atovaquone at or below the FDA-recommended dosaging for prevention or treatment of PCP (discussed below) for one, two, or more days in advance of the beginning of radiation therapy, and then separately dose the oltipraz and/or other Nrf2 activator prior to radiation therapy. The atovaquone and/or other OCR-API and the oltipraz and/or other Nrf2 activator then may continue to be administered throughout the duration of the radiation therapy and then discontinued following the end of radiation therapy.

The composition comprising oltipraz (either recrystallized or formulated as described above in Section A) and/or other Nrf2 activator(s) and an OCR-API may be a fixed combination. In a fixed combination, oltipraz and/or other Nrt2 activator(s) and the OCR-API are present in the same composition. A fixed combination can be used for simultaneous administration of oltipraz and/or other Nrf2 activator(s) and the OCR-API. The two components in a fixed combination are typically intermixed. In such a composition comprising a mixture, the ratio of oltipraz (whether recrystallized or formulated as described in Section A) to OCR-API such as atovaquone can be within a range of from less than 100:1, 100:1 to 50:1, 50:1 to 25:1, 25:1 to 10:1, 10:1 to 5:1, 5:1 to 1:1, 1:1 to 1:5, 1:5 to 1:10, 1:10 to 1:25, 1:25 to 1:50 and 1:50 to 1:100, or more than 1:100.

Dry pharmaceutical compositions provided by this disclosure thus can comprise a mixture of oltipraz (either recrystallized or formulated as described above in Section A) and/or other Nrf2 activator(s) and optionally at least one OCR-API such as atovaquone. As noted above, the oltipraz can be recrystallized oltipraz such as that disclosed in U.S. Pat. No. 4,110,450 or Framroze PCT/IN2016/050197. Alternatively, recrystallized oltipraz such as that produced by U.S. Pat. No. 4,110,450 or Framroze PCT/IN2016/050197 may be further processed and formulated into oltipraz crystal-containing compositions as described in Section A above.

The oltipraz crystal compositions described in Section A above may be used to formulate various kinds of pharmaceutical preparations that may be used alone or in conjunction with an OCR-API. The preparations typically comprise a dry composition as described above. Practically speaking, pharmaceutical compositions comprising the dry composition can comprise any amount of the oltipraz crystals. The amount of the composition will depend on the desired dosage of the oltipraz and the concentration of the oltipraz in the dry composition. In certain embodiments, for example, the dry composition comprises a single dose of up to 5000 mg. e.g., 100 to 500 mg, 500 to 1000 mg, 1000 to 1500 mg, and 1500 to 2000 mg, 2000 to 2500 mg, 2500 mg to 3000 mg, 3000 mg to 4000 mg and 4000 mg to 5000 mg. The dose may thus be from 100 to 5000 mg such as from 500 to 4000 mg, such as from 1000 to 3000 mg e.g. from 1500 to 2000 mg. Single dosage amounts over 5000 mg also may be employed. Within such ranges are exemplary amounts of up to 600 mg of a dry composition as described above, up to 500 mg of a dry pharmaceutical composition, up to 400 mg of a dry pharmaceutical composition, up to 350 mg of a dry composition, or up to 300 mg of a dry composition as described herein. Exemplary amounts within such ranges also include 250 mg, 300 mg, 350, mg, 400 mg, 450 mg, 500 mg, 550 mg, 600 mg, 650 mg, 700 mg, 750 mg, 800 mg, 850 mg, 900 mg, 950 mg and 1000 mg, 1100 mg, 1200 mg, 1300 mg, 1400 mg, 1500 mg, 1600 mg, 1700 mg, 1800 mg, 1900 mg and 2000 mg. As described above, such dry pharmaceutical compositions can comprise from 5% to over 25% of oltipraz crystals. For example, if the dry composition comprises 5% oltipraz crystals, then the foregoing dosages comprise up to 250 mg of oltipraz. If the dry composition comprises 15% oltipraz crystals, then the foregoing dosages comprise up to 750 mg of oltipraz, and if the dry composition comprises 25% oltipraz crystals, then the foregoing dosages comprise up to 1250 mg of oltipraz.

Dry pharmaceutical compositions also may tend to be fairly electrostatic and so including a small amount of one or more pharmaceutically acceptable lubricants. e.g., magnesium stearate or silica oxide, can assist in the process of metering out quantities of the dry composition. Other processing techniques such as granulation, for example, roller compaction, high shear or fluid bed, may also be used to produce larger particles with binders or other pharmaceutical excipients that are more easily processed and still have rapid dissolution and greater solubility.

The dosaging of the OCR-API will at typically follow or be less than the approved dosaging for the particular OCR-API. For example, the USFDA-approved dosaging for atovaquone is either 1,500 mg once daily with food for prevention of PCP and 750 mg twice daily with food for the treatment of PCP. Hence. e.g., a patient could take either a daily or twice-daily dose of atovaquone with food.

In certain embodiments, a dry composition of oltipraz crystals prepared according to Section A above may be re-suspended in water and/or other liquid for oral administration as a liquid composition in a weight:weight ratio, of 1 part of dry composition and an amount of water of from less than 10 parts of water (or other liquid) up to 200 parts or more of water (or other liquid). Within such ranges include. e.g., 1-10, 10-20, 20-30, 30-40, 40-50, 50-60, 60-7, 70-80, 80-90, 90-100, 100-125, 125-150, 150-175, 175-200, or more than 200 parts of water (or other liquid) per part of dry composition. The ratio of dry composition to liquid can therefore be from 1:10 to 1:200 such as from 1:20 to 1:150 e.g. 1:30 to 1:100 such as 1:40 to 1:70 e.g. about 1:50 to 1:60. As noted above, where the composition is prepared using at least one stabilizing agent, the MHD of the crystals in the composition may remain within the target range for a period of time. e.g., at least 1 hour, at least 3 hours, at least 6 hours, at least 12 hours or at least 24 hours, or longer. Further, depending on the combinations of stabilizing agent(s), if any, and bulking agent (if any) and crystal size, the re-suspended composition also may readily dissolve, e.g., with vigorous shaking for less than 15 minutes, less than 10 minutes, less than 5 minutes, less than three minutes, less than 2 minutes less than one minute, or less than 30 seconds, and also may remain substantially homogeneously suspended for a period of time. e.g., for at least 1 hour, at least 3 hours, at least 6 hours, at least 12 hours, or at least 24 hours. A suspension of oltipraz crystals may be deemed to be substantially homogeneous if the concentration of oltipraz in a test sample taken from the top of the liquid composition after a defined period of time (e.g., less than 1 minute, 1 minute, 2 minutes, 5 minutes, 10 minutes, or 15 minutes) comprises a desired minimum target percentage of the original concentration, e.g., at least 85%, 90%, 95% or 98% of the concentration of oltipraz in a sample taken from the liquid composition immediately after the composition is resuspended to form a substantially homogeneous composition. Depending on the particular OCR-API(s) to be co-administered with the oltipraz, a dry composition of the OCR-API such as atovaquone can be included in the dry formulation together with the dry composition of oltipraz crystals described above, and then can be resuspended with water and/or other liquid prior to administration.

Formulations of the pharmaceutical compositions for oral administration also may be presented as a mouthwash, or a carbonated liquid, or an oral spray or aerosol, or an oral ointment, gel, or cream.

In certain embodiments, liquids suitable for formulating compositions comprising oltipraz (e.g., either recrystallized or formulated as described above in Section A) and/or other Nrf2 activator(s), either with or without one or more OCR-APIs such as atovaquone, for oral administration, e.g., buccal administration, may include buccal vehicle components known to the art.

In other embodiments, the oral formulations of compositions comprising oltipraz (e.g., either recrystallized or formulated as described above in Section A) and/or other Nrf2 activator(s), either with or without one or more OCR-APIs such as atovaquone, may be emulsions or suspensions.

Liquid dosage forms useful for oral administration of compositions comprising oltipraz (e.g., either recrystallized or formulated as described above in Section A) and/or other Nrf2 activator(s), either with or without one or more OCR-APIs such as atovaquone, include pharmaceutically acceptable emulsions, microemulsions, suspensions, syrups and elixirs. In addition to the active ingredients, the liquid dosage forms may contain inert diluents commonly used in the art.

Besides inert diluents, the oral compositions can also include adjuvants such as wetting agents, emulsifying and suspending agents, sweetening, flavoring, coloring, perfuming and preservative agents.

Examples of suitable aqueous and nonaqueous carriers that may be employed in the pharmaceutical compositions comprising oltipraz (e.g., either recrystallized or formulated as described above in Section A) and/or other Nrf2 activator(s), either with or without one or more OCR-APIs such as atovaquone, include water, ethanol, polyols.

Compositions for oral administration may include additional components, such as coloring agents, flavoring agents, fragrances, antimicrobial agents, or sweetening agents as further described.

Alternative embodiments of pharmaceutical compositions suitable for oral administration of compositions comprising oltipraz (e.g., either recrystallized or formulated as described above in Section A) and/or other Nrf2 activator(s), either with or without one or more OCR-APIs such as atovaquone include but are not limited to compositions in the form of capsules (including sprinkle capsules and gelatin capsules), sachets, stickpacks, pills, tablets, and lozenges.

One or more additional agents that are generally recognized as safe for administration to humans and can be co-administered together with the oltipraz or oltipraz-OCR-API compositions composition, or co-administered separately as part of a dosing regimen with the oltipraz composition, include N acetylcysteine and/or other antioxidants, BHT, pantothenic acid (vitamin B5) or other agents that enhance glutathione synthesis, glutathione, e.g., for topical administration, Medihoney (for topical administration), curcumin (for topical administration) or other NF-kappaB inhibitors, Mesalamine and/or other anti-inflammatory agents, e.g., for oral or rectal administration compositions, and superoxide dismutase or other compounds that prevent damage from reactive Of (superoxide).

D. Devices for Oral Administration

In certain embodiments, liquid formulations of the compositions comprising oltipraz (e.g., either recrystallized or formulated as described above in Section A) and/or other Nrf2 activator(s), either with or without one or more OCR-APIs such as atovaquone may be prepared and administered using a device that facilitates administration of a single dose of the pharmaceutical composition. Such devices, which are known in the art, can include a cavity or reservoir where a dry composition and a liquid such as water and/or a non-aqueous solvent may be mixed and then administered to the patient via an opening in the device. Typically, such devices comprise a cavity and a compartment that is separate from the cavity, in which compartment a dry powder can reside. At the time of administration, the powder is released from the compartment into the cavity or reservoir. In some devices, this is accomplished by breaking a barrier that separates the compartment from the cavity or reservoir. Thereafter, the powder may be mixed, typically by shaking, with a liquid in the cavity that may have been added earlier or at the time. The cavity is of sufficient size to hold both the dry pharmaceutical composition and a quantity of liquid comprising an amount of water and/or non-aqueous solvent sufficient to permit mixing of the dry pharmaceutical composition to form a liquid composition. The liquid may be added to the container at the time of packaging to create a self-contained product comprising both dry composition and liquid that may be mixed together at the time of administration. Alternatively, the container can contain only a dry pharmaceutical composition and the liquid is then added prior to administration. The liquid may contain flavoring additives as discussed below. Alternatively, other types of packaging that separate the dry and liquid ingredients may be used. For example, the powder and the liquid can be sealed in 2 form-fill-and-seal pouches, either side by side or one on top of the other and separated by a rupturable seal. The person administering the drug would then rupture the seal and mix the contents back and forth between the 2 compartments until dissolved or suspended.

Once the composition is substantially homogeneous (e.g., from the shaking), it is then administered to the patient via an opening in the device created, e.g., by uncoupling a portion of the device to expose the cavity containing the liquid mixture. For example, a portion of the device, e.g., the top, can be removed by unscrewing a threaded portion from another threaded portion of the container to expose the cavity containing the liquid mixture, which then may be administered to the patient or by the patient. Examples of such devices are provided in U.S. Pat. No. 6,148,996. U.S. application 20080202949, and U.S. Pat. No. 3,156,369. Such single-use devices can be employed for orally administering liquid compositions described herein, especially for prophylaxis or treatment of oral mucositis or its symptoms as described below.

The disclosure thus also provides a kit comprising (i) compositions comprising oltipraz (e.g., either recrystallized or formulated as described above) and/or other Nrf2 activator(s), either with or without one or more OCR-APIs such as atovaquone (ii) a device for oral administration of such compositions. The kit optionally further contains instructions for use. The kit optionally could comprise (i) an OCR-API (e.g., atovaquone powder) and a device for administration of the OCR-API, and (ii) an oltipraz-containing composition and a device for administration of the oltipraz-containing composition in the event that the OCR-API and oltipraz-containing compositions are intended to be administered separately.

For such devices, the oltipraz crystal composition may be in a dry form admixed with a dry form of the OCR-API(s), e.g., a dry form of atovaquone. In such instances, the dry composition which can be present together, e.g., in a compartment as described above, is admixed with water and/or other liquid solvent prior to administration (e.g., by exposing the dry composition to the liquid and shaking) as discussed above. Additionally (in the case of more than one OCR-API), or alternatively (in the case of one OCR-API), the OCR-API(s) also can be contained in the liquid portion of the container prior to mixing of the oltipraz or oltipraz-OCR-API composition. Thus, for example, an oltipraz-containing composition such as described above in Section A (with or without an OCR-API mixed in) could be stored in a dry form in a compartment of the device, and a different OCR-API (e.g., atovaquone) could be in the liquid portion of the container.

As another example, a patient could be given multiple of such devices, some of which contain only atovaquone powder, and some of which contain either an oltipraz composition or OCR-API-oltipraz composition as described above in Section C. As discussed below, the atovaquone can be administered either together with, or separately from, the oltipraz composition. Such would be the case if the administration of the atovaquone is to begin 1, 2 3 or more days before the administration of the oltipraz composition. The kit could further provide labeling to indicate which composition is to be taken on which day.

E. Compositions for Topical Administration

In some embodiments, the oltipraz or oltipraz-OCR-API formulations may be suitable for topical administration, and may include any of the constituents outlined below.

Suitable moisturizers for use in the formulations include, but are not limited to, lactic acid and other hydroxy acids and their salts, glycerol, propylene glycol, butylene glycol, sodium PCA, sodium hyaluronate. Carbowax 200, Carbowax 400, and Carbowax 800.

Suitable humectants include, but are not limited to, panthenol, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, sunflower seed oil, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopheryl-succinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, polyquaternium-10 (quaternized hydroxyethyl cellulose), camellia oleifera leaf extract, phytosteryl canola glycerides, shea butter, caprylic/capric triglycerides, punica granatum sterols, ethylhexyl stearate, betaine, behenyl alcohol (docosanol), stearyl alcohol (1-octadecanol). laminaria ochroleuca extract, behenic acid, caproyl sphingosine, caproyl phytosphingosine, dimethicone-divinyldimethicone-silsesquioxane crosspolymer, potassium lactate, sodium hyaluronate crosspolymer, hydrolyzed hyaluronic acid, sodium butyroyl-formoyl hyaluronate, polyglutamic acid, tetradecyl aminobutyroylvalylaminobutyric urea trifluoroacetate, micrococcus lysate, hydrolyzed rice bran protein, glycine soja protein, and 1,3-bis(N-2-(hydroxyethyl)palmitoylamino)-2-hydroxypropane.

The topical compositions also may be delivered transdermally via a patch that is applied over the skin, and such patches are well known in the art.

Persons of skill in the art will recognize other topical delivery compositions and vehicles that may be used.

F. Compositions for Rectal/Colonic Delivery

In certain embodiments, the pharmaceutical compositions described in Section C above, whether including only oltipraz (whether or not formulated as described in Section A above) or including both oltipraz and the OCR-API(s), can be formulated for rectal administration to provide colon-specific delivery using known methods and compositions. Generally speaking, delivery of pharmaceutical composition via rectal administration route can be achieved by using suppositories, enemas, ointments, creams or foams. Suppositories are among the most common rectal dosage forms, and bases are generally fatty in nature, but water-soluble or water-miscible bases can also be utilized. In order to achieve a desirable bioavailability the active ingredient should come in contact with the rectal or colonic mucosa.

Suitable excipients for preparing compositions for rectal administration such as, but not limited to, vehicle, preservatives, surfactants, emulsifiers, mineral oils, propellants, thickening agents, lubricants, preservatives, pH adjusting agents, chelating agents, emollients and/or humectants, permeation enhancers, suspension-forming agents or mucoadhesive agents or combinations thereof. The vehicle may include an aqueous, non-aqueous or a hydro-alcoholic vehicle. Suitable aqueous vehicles which are compatible with the rectal and colonic mucosa, may comprise water soluble alkanols selected from, but not limited to, ethanol, polyalcohols such as a propylene glycol, glycerol, polyethyleneglycol, polypropylene glycol, propylene glycol glyceryl esters and combinations thereof. Non-aqueous vehicles which may be employed in pharmaceutical rectal foam compositions, including but not limited to vegetable oils, such as olive oil; injectable organic esters, such as ethyl oleate and combinations thereof.

Suitable surfactants that may be employed in pharmaceutical compositions for rectal administration, e.g. anionic surfactants, non-ionic surfactants, cationic surfactants, and amphoteric (zwitterionic) surfactants. Anionic surfactants may include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinate, disodium lauryl sulfosuccinate, dianunonium lauryl sulfosuccinate, tetrasodium N-(1,2-dicarboxyethyl)-N-octadecylsulfosuccinate, diamyl ester of sodium sulfosuccinic acid, dihexyl ester of sodium sulfosuccinic acid, dioctyl esters of sodium sulfosuccinic acid, docusate sodium, and combinations thereof.

Nonionic surfactants may include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA. PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethylene oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof.

Amphoteric surfactants may include, but are not limited to, sodium N-dodecyl-beta-alanine, sodium N-lauryl-beta-iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, oleamidopropyl belaine, coco dimethyl sulfopropyl belaine, stearyl dimethyl sulfopropyl betaine, lauryl dimethyl sulfoethyl betaine, lauryl bis-(2-hydroxyethyl)sulfopropyl betaine, and combinations thereof.

Cationic surfactants may include, but am not limited to, behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowedimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquaternium, stearalkonium chloride, stearyl dimethylbenzyl ammonium chloride, stearyl trimethyl ammonium chloride, trimethylglycine, and combinations thereof.

Suitable thickening agents or viscosity modifying agents which may be employed in the pharmaceutical composition for rectal administration include, but are not limited to, carboxymethyl cellulose, polyoxyethylene-polyoxypropylene copolymers, xanthan gum, agar, guar gum, hydroxyethyl cellulose, hydroxypropyl cellulose, methyl cellulose and combinations thereof.

Alternatively, colonic absorption can be accomplished through oral administration of compositions including only oltipraz (whether or not formulated as described in Section A above) or including both oltipraz and the OCR-API(s), which compositions are designed to release the active oltipraz in the colon. Such compositions can be in an oral dosage form. e.g., a pill, capsule or liquid, that provides delayed release until the dosage form is in the colon

G. Compositions and Devices for Inhalation Administration

In other embodiments, pharmaceutical compositions described above in Section C including only oltipraz (whether or not formulated as described in Section A above) or including both oltipraz and the OCR-API(s), may be delivered via the respiratory tract by providing the composition in inhalable form, e.g., in an inhaler device, either in dry powder form or in a liquid carrier. For example, inhalable compositions can comprise the active ingredient in dry powder compositions provided in dry powder inhalers. See, e.g., WO2014177519 and US20140065219. Alternatively, inhalable compositions can comprise the active ingredient in a liquid carrier such as ethanol. See, e.g., EP2536412 A2.

The disclosure thus also provides a kit comprising (i) a pharmaceutical composition as described in Section C above, and (ii) a device for administering such composition by inhalation. The kit optionally further contains instructions for use.

H. Methods of Treating

In certain embodiments, the pharmaceutical compositions and devices for oral administration described in Sections C through G above comprising an OCR-API such as atovaquone in combination with oltipraz or formulated oltipraz crystal compositions as described herein may be used for treating a human or non-human animal patient in need. The patient typically will be a human patient, although the pharmaceutical compositions of this disclosure can be used for treating non-human animals, e.g., for veterinary uses. The compositions of this disclosure may be used for preventing or treating a wide variety of diseases and conditions, including diseases and conditions for which treatment with oltipraz is known. Examples of such diseases and conditions include mucositis, HIV, cancers, hepatitis (including HBV and HCV), keratin-based skin diseases, including skin blistering and epidermolysis bullosa simplex and related diseases, inflammatory disorder or disease (including endothelial dysfunction and cardiovascular disease), cachexia, weight loss, sepsis, contrast-induced nephropathy, diabetes, obesity. PCOS, steatosis, hyperlipidemia, and hypertension, chronic kidney disease, pulmonary fibrosis, hypoxic conditions, chemical-induced lung injury, respiratory distress disorder, anon gap acidosis, nephritis, lupus, interstitial lung disease, graft dysfunction, hepatitis, acute kidney injury, noise-induced hearing injuries, poison ingestion, retinopathy, neurotoxicity, cancer-induced injury such as ototoxicity, respiratory infections, autism, conditions involving vasospasm, and conditions considered treatable by provision of n-acetylcysteine, injectable reduced glutathione, or a known intracellular glutathione enhancing agent.

The pharmaceutical compositions and devices for administration comprising either (i) an OCR-API such as atovaquone in combination with oltipraz or formulated oltipraz crystal compositions as described in Sections C through G, or (ii) oltipraz or formulated oltipraz crystal compositions as described herein, and/or other Nrf2 activator(s) either with or without one or more OCR-APIs and/or other pharmaceutically active ingredients, also can be used to prevent, treat, lessen the symptoms, and/or decrease the injury associated with ischemia/reperfusion injury. Such injury occurs, for example when cross-clamping the aorta for vascular repairs, myocardial infarction, or a variety of vascular procedures in which a clot is removed, including stroke. Such injury also can occur during organ transplant surgery. One or more atovaquone or other OCR-APT can provide some protection for the ischemic cells, while the oltipraz and/or other Nrf2 activator(s) can protect the cells from oxidative damage when reperfusion is established. Where time is of the essence, e.g., in the case of stroke or myocardial infarction, co-administration of the OCR-API and oltipraz or other Nrf2 activator(s) may comprise giving both at substantially the same time. Where there is an opportunity to prepare the patient for the procedure, then the co-administration of the OCR-API and oltipraz or other Nrf2 activator(s) may comprise giving the drugs at different times. OCR-APIs that may be useful in combination with oltipraz and/or other Nrf2 activator(s) to prevent, treat, lessen the symptoms, and/or decrease the injury associated with ischemia/reperfusion injury include but are not limited to meclizine, nimorazole, metformin, phenformin, antimycin A, pyrvinium, berberine, niclosamide, acriflavinium, sorafenib, emetine, plicamycin, suloctidil, pentamidine, amsacrine, irinotecan, itraconazole, mitomycin, hydroxyprogesterone, cyclosporine, fenofibrate, analogues of ubiquinone such as atovaquone, and combinations thereof. Nrf2 activators that may be used include but are not limited to sulphoraphane, phenethyl isothiocyanate, oltipraz, curcumin, resveratrol, fumaric acid and its esters, and synthetic oleanane triterpenoids.

One or more OCR-APIs, either alone or with oltipraz and/or other Nrf2 activator(s), or oltipraz or formulated oltipraz crystal compositions as described herein, either with or without one or more OCR-APIs and/or other pharmaceutically active ingredients, also can be useful in a composition for storage, transport and/or perfusion of organs in preparation for transplantation in order to lessen the degradation of organ tissue following removal from the organ donor, and/or to prevent, treat, lessen the symptoms, and/or decrease reperfusion injury during or following transplantation. OCR-APIs that may be useful for such compositions include but are not limited to meclizine, nimorazole, metformin, phenformin, antimycin A, pyrvinium, berberine, niclosamide, acriflavinium, sorafenib, emetine, plicamycin, suloctidil, pentamidine, amsacrine, irinotecan, itraconazole, mitomycin, hydroxyprogesterone, cyclosporine, fenofibrate, analogues of ubiquinone such as atovaquone, and combinations thereof. Nrf2 activators that may be used include but are not limited to sulphoraphane, phenethyl isothiocyanate, oltipraz, curcumin, resveratrol, fumaric acid and its esters, and synthetic oleanane triterpenoids. Alternatively, the composition for storage, transport and/or perfusion of organs may contain one or Nrf2 activators such oltipraz (e.g., either recrystallized or formulated crystal compositions as described herein) and/or the others described above, but not an OCR-API.

Typically, the pharmaceutical composition(s) is/are provided to the patient in an effective amount. The term “effective amount” is used herein to refer to an amount of the therapeutic composition sufficient to produce a significant biological response (e.g., a significant decrease in inflammation). Actual dosage levels of the Nrf2 activators such as oltipraz, or oltipraz and OCR-API(s) such as atovaquone in a therapeutic composition can be varied so as to administer an amount that is effective to achieve the desired therapeutic response for a particular subject and/or application. Of course, the effective amount in any particular case will depend upon a variety of factors including formulation, route of administration, combination with other drugs or treatments, severity of the condition being treated, and the physical condition and prior medical history of the subject being treated.

As used herein, the term “subject” includes both human and animal subjects, and thus veterinary therapeutic uses are provided in accordance with this disclosure. The terms “treatment” or “treating” relate to any treatment of a condition of interest (e.g., mucositis, an inflammatory disorder or a cancer), including but not limited to prophylactic treatment and therapeutic treatment. As such, the terms “treatment” or “treating” include, but are not limited to: preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest.

The compositions and devices described in Sections C through G are suitable for treating patients who are suffering from mucositis or who will undergo a treatment such as radiation treatment or chemotherapy that can lead to mucositis, e.g., in the oral cavity (including in the buccal cavity), in the alimentary canal, in the colon and/or rectum, and/or on the skin. Where the mucositis is oral mucositis, the compositions and devices of Sections C and D may be preferred. Such patients, e.g., may be undergoing, or about to undergo chemotherapy and/or radiation therapy. e.g., radiation treatment in the head and neck area, or to another area of the body.

The compositions and devices of an OCR-API such as atovaquone in combination with one or more Nrf2 activators such as oltipraz or formulated oltipraz crystal compositions as described in Sections C through G may be used to accomplish one, more than one, or all of the following beneficial effects on human or non-human animal patients, i.e., (i) prophylactically prevent or delay the onset of mucositis, including oral mucositis (e.g., inflammation of the mucosa), (ii) treat existing mucositis, including oral mucositis (iii) alleviate symptoms associated with mucositis, including oral mucositis (iv) reduce or lessen the severity of existing mucositis, including oral mucositis (v) hasten the cure or healing of mucositis, including oral mucositis (vi) reduce the incidence and/or duration of mucositis, including oral mucositis, e.g., mild, moderate and severe oral mucositis, (vii) prophylactically prevent or delay the onset of weight loss or cachexia by a patient with oral mucositis. (viii) reduce the amount of weight loss or cachexia experienced by a patient with oral mucositis, and/or (ix) increase the ability of a patient with oral mucositis to take food by mouth. Such compositions also may be used for the prevention and/or treatment of patients with dysphagia (difficulty swallowing). e.g., cancer patients, or to delay the onset of dysphagia or lessen the severity of dysphagia, e.g., in cancer patients. Such compositions also may be used for the prevention and/or treatment of patients with xerostomia (the subjective feeling of oral dryness), or to delay the onset of xerostomia, lessen the severity of xerostomia, and/or reduce the incidence of moderate-to-severe xerostomia. In certain embodiments, the single-use devices described above may be used for administration of liquid compositions for accomplishing one, more than one, or all of the above relating to oral mucositis, dysphagia and xerostomia. Advantageously, formulations are also non-irritating, well-tolerated, palatable (if orally administered), non-cytotoxic, weakly or non-sensitizing, non-sensitizing.

This disclosure thus provides methods for treating mucositis, comprising administering (in the case of a combined composition), or co-administering (in the case of separate compositions), to a patient in need thereof a therapeutically effective amount of a pharmaceutical composition or compositions as described in Sections C through G herein. The disclosure also provides pharmaceutical compositions as described in Section C herein for use in the treatment of mucositis. The disclosure also provides the use of a pharmaceutical composition as described herein in the manufacture of a medicament for the treatment of mucositis. The administration of the pharmaceutical composition to a patient may be an oral administration, including buccal administration.

The term “co-administer” or “co-administration” simply means administering two (or more) compositions to a patient in order to provide the patient with a therapeutic effect that is the result of having administered both compositions. Co-administration denoted administering the medications in combination, but not necessarily simultaneously. The methods of administration described herein can represent a treatment regimen of a predetermined duration. e.g., 1 week, 2 weeks, 3 weeks, 1 month, 2 months, 3 months, 4 months, 5 months, 6 months, or longer. Compositions according to this disclosure can be applied or administered once daily, twice daily, three times daily, or as needed. In situations where the patient is undergoing chemotherapy and or radiation therapy, the composition(s) may be administered or co-administered prior to a treatment once or multiple times in order to prepare the patient for the treatment. For example, one or both of the composition(s) may be administered or co-administered within 1 hour, within 3 hours, within 6 hours, within 12 hours, within 24 hours, within 24 to 48 hours, within 48 to 72 hours, or more than 72 hours before the treatment. In embodiments, one of the compositions, e.g., the OCR-API may be administered further ahead of the treatment in order to achieve the systemic OCR affect, as compared to the oltipraz, which may be administered closer to the time treatment. The length of time that each will be administered of treatment ahead will generally depend on when each member of the combination will provide the patient with the greatest therapeutic effect at, near and/or following the time of treatment. The determination of such interval(s) will be made by the physician based on the circumstances and the known dosing frequency of the OCR-API. Additionally, or alternatively, the dosage may be administered after a treatment, e.g., within 1 hour, within 3 hours, within 6 hours, within 12 hours, within 24 hours after the treatment, or more than 24 hours after the treatment. Again, the determination of such interval(s) will be made by the physician based on the circumstances and the known dosing frequency of the OCR-API. Where the OCR-API is atovaquone, the atovaquone can be administered prior to the start of the administration of the oltipraz composition. For example, atovaquone can be administered one time or multiple times to the patient 1, 2, 3 or more days before administering the oltipraz composition. Co-administration of the atovaquone and oltipraz then can be continued through the duration of the treatments, e.g., on a daily basis. As noted above, the term co-administration does not require that the different compositions need to be taken together or even near each other in time.

For example, as discussed above, a patient could be administered a package or kit comprising multiple devices as described above in Section D. A first group of the devices can contain only atovaquone and/or other OCR-API and would be administered to the patient, with food, one or more days in advance of beginning radiation therapy. For example, the first group could comprise two devices, one of which is to be taken on each of the two days preceding the start of radiation therapy. A second group of the devices could contain compositions described above in Section C comprising both oltipraz and/or other Nrf2 activators and atovaquone, which then would be administered daily beginning on the first day of radiation therapy or administered periodically during the radiation therapy, for the duration of the treatments. Alternatively, if the atovaquone and oltipraz compositions are to be administered separately, then the kit would contain some devices that contain only atovaquone and some devices that contain only oltipraz including in the compositions described in Section C above. Alternatively, for example, the patient could administer the OCR-API using the standard packaging for such drug, e.g., for Mepron (atovaquone), and use a device as discussed herein for administering the oltipraz-containing composition.

Where liquid compositions are administered, the composition may be administered orally or parenterally, e.g., by subcutaneous, intramuscular, intrasternal, or intravenous injection. Where oral administration is employed, the liquid composition simply may be swallowed, or it may be administered by a “swish and swallow” regimen or a “swish and spit” regimen. By administering the composition orally in a liquid form to a patient with oral mucositis, the compositions may provide a therapeutic dosage of oltipraz at the site of administration, which can provide a therapeutic benefit in terms of the mucositis as described above, i.e., it may prophylactically prevent the onset of mucositis, treat existing mucositis, alleviate symptoms associated with mucositis (e.g., inflammation of the mucosa), reduce or lessen the severity of existing mucositis, and/or hasten the cure or healing of mucositis. In such cases, liquid compositions comprising an ingredient with a negative charge. e.g., a cationic surfactant or polymer such as Eudragit RL, may provide a further advantage by virtue of providing an adherence or association with the mucosa of the mouth, which tends to have a positive charge. The physical and chemical properties of embodiments of the compositions described herein can impart characteristics to the formulation such as stability, delivery of the active agent to the mucosal membrane, and ease of administration.

As noted above, oltipraz compositions as described herein may be co-administered with other therapeutic agents, either together or separately as part of a therapeutic regimen. Such agents include N acetylcysteine and/or other antioxidants, pantothenic acid (vitamin B5) or other agents that enhance glutathione synthesis, glutathione, e.g., for topical administration, Medihoney (for topical administration), curcumin (for topical administration) or other NF-kappaB inhibitors, Mesalamine and/or other anti-inflammatory agents, e.g., for oral or rectal administration compositions, and superoxide dismutase or other compounds that prevent damage from reactive O₁⁻ (superoxide).

### EXAMPLES

Certain embodiments of this disclosure are further illustrated by the following examples, which should not be construed as limiting in any way.

### Example 1: Method for Manufacturing an Oltipraz Composition

A pharmaceutical composition comprising oltipraz, stabilizing agents polysorbate 80 and Eudragit RL, and a bulking polymer, polyvinylpyrrolidone vinylacetate (PVP-VA64), was manufactured by the following steps.

In an appropriate sized container with agitator, formulation components were added in the following order: stabilizing polymer, purified water, polysorbate 80, then oltipraz. The mixture was stirred to create a homogeneous suspension vehicle. The composition of the suspension vehicle prior to milling is shown in Table 2. The suspension vehicle was milled in a temperature-controlled grinding chamber (such as a Dyno-mill, model KDL) with 0.5 mm yttrium-stabilized zirconium oxide spheres as a grinding media. A list of additional mill parameters is shown in Table 3. Total milling time of the suspension was 270 minutes, determined based on a target mean residence time of 7 minutes in the grinding chamber (see Equation 1). The MHD of the crystals/particles in the milled suspension was measured by dynamic light scattering (DLS) performed as described above and was 330 nm.

The milled suspension was transferred to a new, appropriate sized solution tank, bulking polymer PVP-VA64 was added, and then additional purified water to dilute the suspension to 28% total solids. The final suspension composition shown in Table 4 was then stirred for at least 30 minutes. The suspension was spray dried with a Niro PSD-1 spray dryer using parameters shown in Table 5. Spray dried powder was collected in a cyclone.

TABLE 2
Composition of Suspension Vehicle

| | | Composition (weight |
|---|---|---|
| Component | Function | percent of suspension) |
| Eudragit RL | Stabilizing agent | 4.3 |
| Polysorbate 80 | Stabilizing agent | 2.1 |
| Oltipraz | Active | 8.6 |
| Water, USP Purified | Solvent | 85.0 |

TABLE 3
Parameters Used with the Dyno-mill KDL

| Parameter | Value | |
|---|---|---|
| Chamber size | 0.6 | L |
| Agitator Paddles | 64 | mm |
| Gap size | 0.2 | mm |
| Rotor Speed | Approximately 3000 rpm (belt | |
| | position 3) | |
| Mill mode | Continuous | |
| Grinding media volume | 2000 | g |
| Suspension Temperature (Reservoir) | 2.0-40.0° | C. |
| Suspension Temperature (Mill outlet) | 2.0-40.0° | C. |
| Suspension Flow Rate | 500 | mL/min |
| Mill Run Time | 270-300 | minutes |

### Example Calculation for Total Required Milling Time of Suspension Vehicle.

$$\frac{Working chamber volume}{F} * total mill time = 7 minutes$$

$$where F = \frac{total suspension mass}{suspension density}$$

Working chamber volume was defined as the empty chamber volume minus the volume of the grinding media.

TABLE 4
Composition of Spray Suspension

| | | Composition |
|---|---|---|
| Component | Function | (% of suspension) |
| Milled Suspension (from Table 1) | — | 48.7 |
| PVP-VA 64 | Bulking polymer | 20.7 |
| Water, USP Purified | Solvent | 30.6 |

TABLE 5
Spray Drying Process Conditions on a PSD-1 Scale Spray Dryer

| Process Condition | Value |
|---|---|
| Atomizer | Spray Systems 2-fluid 2850/120 |
| Atomization gas pressure (psig) | 20 |
| Drying-gas inlet temperature (° C.) | 105 |
| Drying-gas outlet temperature (° C.) | 50 |
| Solution flow rate (g/min) | 35 |
| Drying-gas flow rate (g/min) | 1850 |

The spray dried powder was analyzed to confirm the powder re-suspended in water within 2 minutes, and the resulting oltipraz milled crystal size was similar to the original crystal size achieved during the milling step. Two tests were performed: first, the powder was re-suspended in water at an oltipraz concentration of 5 mg/mL and the time to uniform suspension by visual observation was recorded. Second, the resulting crystal size of the suspension was measured by DLS. The spray dried powder re-suspended in water with vigorous shaking within 2 minutes, and the resulting suspension crystal size was 370 nm which was similar to the original milled suspension crystal size.

### Example, 2: Stability Testing of an Oltipraz Composition

Samples of a lot of a dry oltipraz composition similar to that prepared in Example 1 were subjected to stability testing for three months at 5° C., 25° C. and 60% relative humidity (RH), and 40° C. and 75% RH. The samples (10 g) were contained inside an LDPE (low density polyethylene) pouch, which was subsequently placed inside of a foil bag. Desiccant (1 g) was put in the foil bag, and then the foil bag was hermetically heat sealed. Results were as follows:
Powder is still same intense orange.
Flowability was poor due to static, as expected. There is no clumping at any condition.
Re-suspension was performed at 5 mg/mL. It was very fast in that the powder re-suspended fully in less than 15 seconds of shaking.
DLS performed immediately after re-suspension showed particle size of re-suspended crystals remained less than 600 nm (see Table 6 below).
At 3-month time point, 24-hour stability of suspension was tested. After initial test, suspension was left on counter 24 hours. Minor settling occurred during that time, but a 15 second shake re-suspended all. DLS showed particle size had not changed.
Potency results were as expected, within error.
Glass transition temperature and crystalline melt temperature of the spray dried crystals were unchanged at 3-months.

TABLE 6
Z-Average Particle Size (nm) by Intensity of Oltipraz
Crystal Suspension in Water (average of n = 2)

| | | | | 3 Month + 1 day |
|---|---|---|---|---|
| Condition | t = 0 | 1 Month | 3 Month | suspended |
| 5° C. | 370 | 422 | 377 | 299 |
| 25° C. and 60% RH | 370 | 412 | 325 | 302 |
| 40° C. and 75% RH | 370 | 393 | 324 | 314 |

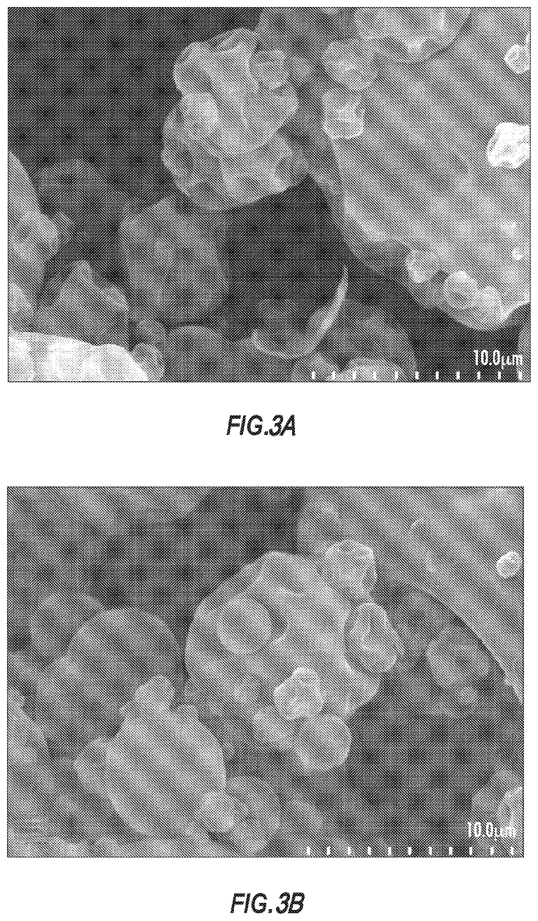

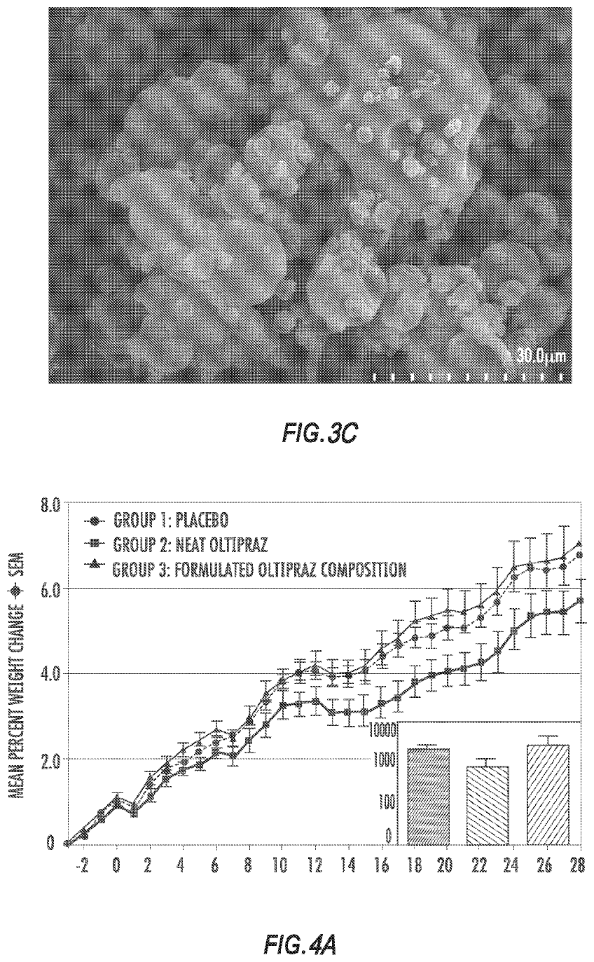

FIG. 3a is a SEM image at 5000× magnification of the dry composition comprising oltipraz at t=0. FIG. 3b is a SEM image at 5000× magnification of the dry composition after stability testing for three months at 40° C. and 75% RH. FIG. 3c is a SEM image at 1500× magnification of the dry composition after stability testing for three months at 40° C. and 75% RH. As can be seen from the figures, particle morphology did not change over time under the test conditions. The particles are still raisin-like to spherical particles with no evidence of crystal growth or particle fusing.

### Example 3: Study for the Assessment of Oltipraz Composition for the Treatment of Oral Mucositis Induced by Acute Radiation in Hamsters

Twenty-four (24) male Syrian Golden Hamsters were used in the study. Mucositis was induced by giving an acute radiation dose of 40 Gy directed to the left buccal cheek pouch on Day 0. Mucositis was evaluated clinically starting on Day 6 and continuing on alternate days until Day 28. Placebo, recrystallized (neat) oltipraz, or a formulated oltipraz composition (described below) at a concentration of 5 mg/mL (based on the amount of crystals in the suspension) was administered by topical application of 0.2 mL directed to the left cheek pouch, twice daily (BID; 1 mg/dose; 2 mg/day) from Days −3 (first dose prior to irradiation) to Day 28.

The formulated oltipraz composition was prepared generally according to the process described in Example 1 and contained 16.7% wt/wt of nanomilled oltipraz crystals (MHD<350 nm) that has been formulated with Eudragit RL, Tween 80 and PVP-VA64 and spray-dried. The neat oltipraz was recrystallized oltipraz prepared according to the process disclosed in WO2016207914.

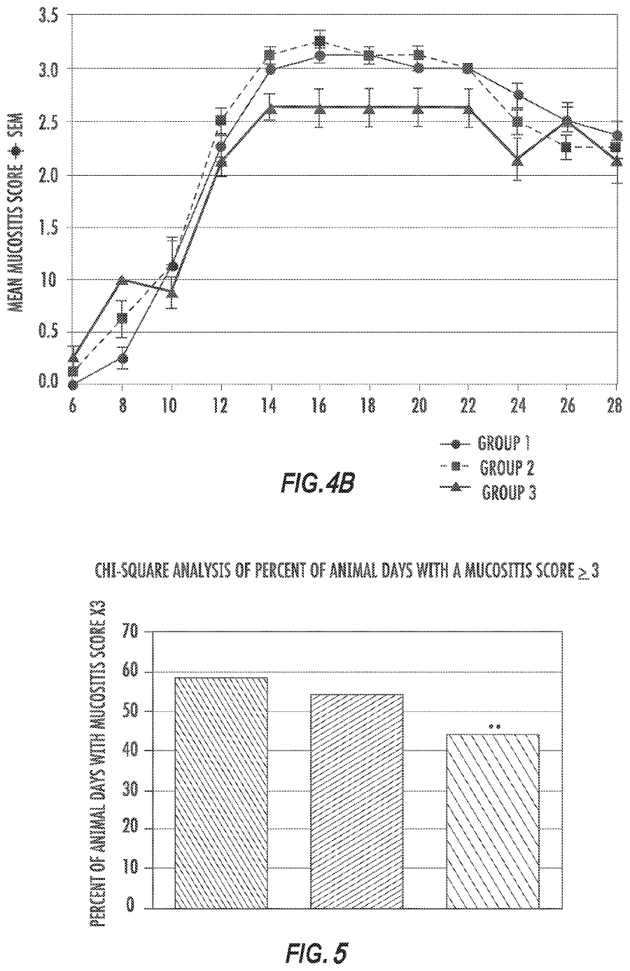

Results and Conclusions
There were no animal deaths at any time during this study.
There were no significant differences in overall mean percent weight change between the placebo control group and the treatment groups from Day −3 to 28, although animals dosed with the formulated oltipraz composition gained substantially more weight and at a significantly faster rate than the animals that were administered neat oltipraz (FIG. 4A), indicating a biological difference in the level of activity.
The maximum mean mucositis score observed in the placebo group was 3.13±0.09 and occurred on Day 16. Animals dosed with neat oltipraz (Group 2) experienced peak mean mucositis score on Day 16 at 3.25±0.11. Animals dosed with the formulated oltipraz composition (Group 3) experienced peak mean mucositis score of 2.63±0.13 and first occurred on Day 14.
Mean daily blind mucositis scores are shown in FIG. 4B. Animals administered Placebo (Group 1) and animals administered neat oltipraz tracked closely together. The maximum mean mucositis score observed in the vehicle group was 3.13±0.09 and occurred on Day 16. Animals dosed with neat oltipraz (Group 2) experienced peak mean mucositis score on Day 16 at 3.25±0.11. In contrast, animals administered the formulated oltipraz composition (Group 3) displayed a substantially and observably reduced mucositis compared to animals administered Placebo (Group 1) or neat oltipraz (Group 2). Supporting this observation, animals receiving the formulated oltipraz composition (Group 3) displayed a peak mean mucositis score of only 2.63±0.13 on Day 16.
Over the course of the study, the percentage of animal days with an ulcerative mucositis (score of ≥3) in the placebo Group was 58.33%. In contrast, the percentage of animal-days with a score of ≥3 was dramatically lower for animals in administered the formulated oltipraz composition (43.75%; p=0.006).

Weight Change

The mean daily percent body weight change data are shown in FIG. 4A for animals in all groups. All animals gained weight steadily over the course of the study (Days −3 to 28), however, animals administered neat oltipraz (Group 2) gained weight at a slower rate than those administered placebo (Group 1) or those administered formulated oltipraz composition (Group 3), suggesting that administration of neat oltipraz may negatively impact weight gain. There were no significant differences in cumulative mean percent weight change between groups in comparing the area under the body weight versus time curve (AUC) analysis followed by evaluation with one-way ANOVA and Holm-Sidak's multiple comparisons test (inset), although as shown in in FIG. 4A, the overall percentage weight change for the animals administered neat oltipraz (Group 2) was substantially less than rate than those administered placebo (Group 1) and lower still as compared against those administered formulated oltipraz composition (Group 3). The percentage rate of weight change for animals administered neat oltipraz was substantially less than the rate for those administered placebo or formulated oltipraz.

Mucositis Scoring

Mucositis was scored visually by comparison to a validated photographic scale, ranging from 0 for normal, to 5 for severe ulceration (clinical scoring). In descriptive terms, this scale is defined as described in Table 7 below:

TABLE 7

| Score | Description |
|---|---|
| 0 | Pouch completely healthy. No erythema or vasodilation. |
| 1 | Light to severe erythema and vasodilation. No erosion of mucosa. |
| 2 | Severe erythema and vasodilation. Erosion of superficial aspects |
| | of mucosa leaving denuded areas. Decreased stippling of mucosa. |
| 3 | Formation of off-white ulcers in one or more places. Ulcers may |
| | have a yellow/gray color due to pseudomembrane. Cumulative size |
| | of ulcers should equal less than or equal to ¼ of the pouch. |
| | Severe erythema and vasodilation. |
| 4 | Cumulative seize of ulcers should equal about ½ of the pouch. |
| | Loss of pliability. Severe erythema and vasodilation. |
| 5 | Virtually all of pouch is ulcerated. Loss of pliability (pouch can |
| | only partially be extracted from mouth). |

### Duration of Ulcerative Mucositis

A mucositis score of 3 or greater indicates ulcerative mucositis, a clinically significant threshold. To quantify the clinical significance of differences observed between the control and treatment groups animal-days with mucositis scores ≥3 and <3 were compared between groups using chi-square analysis. The results of this analysis are shown in Table 8 and FIG. 5 for the entire study duration (through Day 28). Over the course of the study (Table 8, FIG. 5), the percentage of animal days with a score of ≥3 in the vehicle Group was 58.33%. The percentage of days with a score of ≥3 was dramatically and statistically lower for animals in Group 3 in comparison to the vehicle Group (Group 1; p<0.01).

Table 8 below provides a chi-square analysis of percent of animal days with a mucositis score ≥3. To examine the levels of clinically significant mucositis, as defined by presentation with open ulcers (score ≥3), the total number of days in which an animal exhibited an elevated score was summed and expressed as a percentage of the total number of days scored for each group. Statistical significance of observed differences was calculated using chi-squared analysis.

TABLE 8
Chi-Square Analysis of Percent of Animal Days with a Mucositis Score ≥3

| | | | Total | | Chi Sq vs. | |
|---|---|---|---|---|---|---|
| Treatment | Days ≥3 | Days <3 | Animal Days | % Days ≥3 | Vehicle | P Value |
| Group 1: Placebo | 112 | 80 | 192 | 58.33% | — | — |
| Group 2: Neat oltipraz | 104 | 88 | 192 | 54.17% | 0.519 | 0.471 |
| Group 3: Formulated | 84 | 108 | 192 | 43.75% | 7.597 | 0.006 |
| oltipraz composition | | | | | | |

FIG. 5 provides a graph of the percent of animal days with mucositis scores 3 for the entire study duration. To examine the levels of clinically significant mucositis, as defined by presentation with open ulcers (a score of ≥3), the total number of days in which an animal exhibited an elevated score was summed and expressed as a percentage of the total number of days scored for the entire study duration (Day 6-28). Statistical significance was evaluated using the Chi-square test in comparison to Vehicle Control; The statistical significance for the Group 3 results was p<0.01.

Mucositis Severity

An analysis of the severity of mucositis was performed using the Mann-Whitney rank sum analysis to compare the visual mucositis scores for Groups 2 and 3 to the vehicle control group (Group 1) on each day of the analysis. The results of this analysis are shown in Table 9 below. In this analysis, 2 consecutive days of significant reduction in the mucositis score are generally required before it is regarded as clinically meaningful. Animals dosed with the formulated oltipraz composition (Group 3) demonstrated four instances of significant improvement in mucositis scores compared to the vehicle control group including a stretch of four consecutive days of statistically significant improvement (Days 14-18) compared to animals administered Placebo (Group 1).

TABLE 9
Comparison of Daily Mucositis Scores.

| | Rank Sum Analysis by Day | | | | | | | | | | | |
|---|---|---|---|---|---|---|---|---|---|---|---|---|
| Group | 6 | 8 | 10 | 12 | 14 | 16 | 18 | 20 | 22 | 24 | 26 | 28 |
| Placebo vs. | 0.4839 | 0.1539 | 0.9181 | 0.2734 | 0.4839 | 0.6539 | 1.0 | 0.4839 | 1.0 | 0.2734 | 0.2734 | 0.7043 |
| Neat Oltipraz | | | | | | | | | | | | |
| Placebo vs. Formulated | 0.1012 | <0.0001 | 0.6774 | 0.7430 | 0.0177 | 0.0321 | 0.0321 | 0.1012 | 0.1012 | 0.0242 | 0.7224 | 0.4320 |
| Oltipraz Composition | | x, y | | | x, z | x, z | x, z | | | x, z | | |

The significance of group differences observed in daily mucositis scores was determined using the Mann-Whitney rank sum test. This nonparametric statistic is appropriate for the visual mucositis scoring scale. The p-values for each calculation are shown. “x” denotes significant difference in mucositis scores. “y” denotes increase in comparison to vehicle Group (improvement). “z” denotes decrease.

Percent of Animals with Ulcerative Mucositis by Day

The percentage of animals in each group with ulcerative mucositis at each day of evaluation is shown in Table 10. This evaluation was intended to clarify which days of treatment had its maximal impact on the course of ulcerative mucositis. Fewer animals displayed ulcerative mucositis when administered the formulated oltipraz composition (Group 3) over ten consecutive day (Days 14-24) in comparison to the animals receiving Placebo (Group 1).

TABLE 10
Percent of Animals with Ulceration by Day with Mucositis Score ≥3.

| | Percent Ulceration by Day (Score ≥3) | | | | | | | | | | | |
|---|---|---|---|---|---|---|---|---|---|---|---|---|
| Group | 6 | 8 | 10 | 12 | 14 | 16 | 18 | 20 | 22 | 24 | 26 | 28 |
| Group 1: Placebo | 0.0 | 0.0 | 12.5 | 25.0 | 100.0 | 100.0 | 100.0 | 100.0 | 100.0 | 75.0 | 50.0 | 37.5 |
| Group 2: Neat Oltipraz | 0.0 | 0.0 | 0.0 | 50.0 | 100.0 | 100.0 | 100.0 | 100.0 | 100.0 | 50.0 | 25.0 | 25.0 |
| | | | z | y | | | | | | z | z | z |
| Group 3: Formulated | 0.0 | 0.0 | 0.0 | 25.0 | 62.5 | 75.0 | 75.0 | 75.0 | 75.0 | 37.5 | 62.5 | 37.5 |
| Oltipraz Composition | | | z | | z | Z | z | z | z | z | y | |

To examine the levels of clinically significant mucositis, as defined by presentation with open ulcers (score ≥3), the percentage of animals from each treatment group that exhibited an open ulcer on each day of the study was determined. “y” denotes an increase in comparison to vehicle Group, “z” denotes decrease (improvement). The results show an improvement in the ulceration scores for the formulated oltipraz composition as compared to either the neat oltipraz or placebo. The results of day 26 appears to have been due to one animal flare from day 24 and the result of day 28 is likely due to the difference in a single animal score.

Conclusions
There were no animal deaths at any time during this study.
There were no significant differences in overall mean percent weight change between the placebo control group and the treatment groups front Day −3 to 28, although as shown in in FIG. 4A, the overall percentage weight change for the animals administered neat oltipraz (Group 2) was substantially less than rate than those administered placebo (Group 1) and lower still as compared against those administered formulated oltipraz composition (Group 3).
The maximum mean mucositis score observed in the placebo group was 3.13±0.09 and occurred on Day 16. Animals dosed with neat oltipraz (Group 2) experienced peak mean mucositis score on Day 16 at 3.25±10.11. Animals dosed with the formulated oltipraz composition (Group 3) experienced peak mean mucositis score of 2.63±0.13 and first occurred on Day 14.
Over the course of the study, the percentage of animal days with an ulcerative mucositis (score of ≥3) in the placebo Group was 58.33%. In contrast, the percentage of animal-days with a score of ≥3 was dramatically lower for animals administered the formulated oltipraz composition (43.75%; p=0.006)

### Example 4: Qualitative Visual Assessment of Oltipraz Compositions

As noted above, the stability of the oltipraz crystals in an aqueous suspension can be assessed in 3 ways. First, they can be assessed by DLS to determine whether there is an increase in the MHD. Second, the potency (and thus the stability) of the suspension can be can be determined by sampling the top of the suspension, making sure not to mix any precipitate back into the suspension. The concentration of drug in the suspension should not decrease by a predetermined amount in a given period. e.g., by more than a predetermined amount e.g., 1%, 2%, 5%, 10%, 15% or 20% in a period selected from 1 minute, 5 minutes, 15 minutes, 30 minutes, 45 minutes. 1 hour, 2 hours, 6 hours, 12 hours and 24 hours.

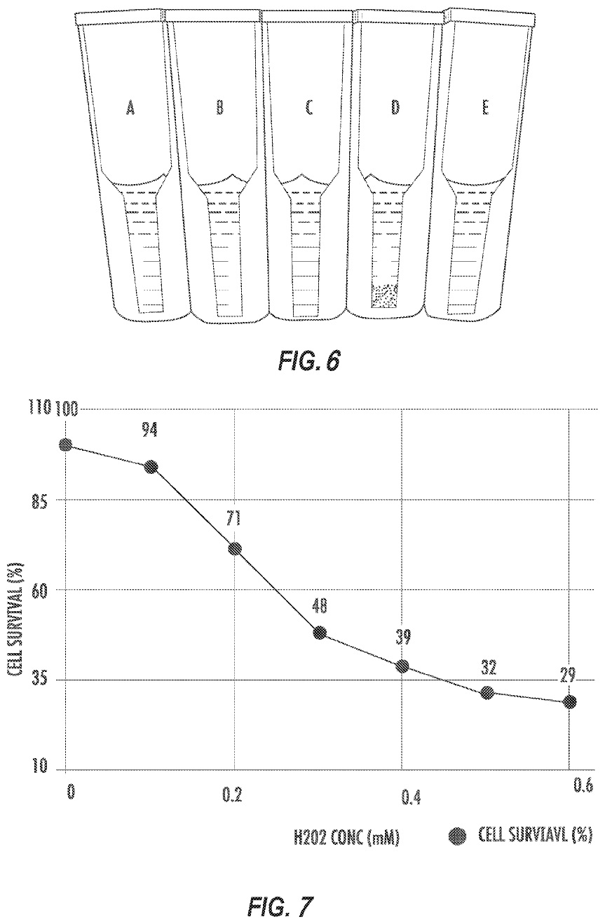

The third way is by a qualitative visual assessment. With a substantially stable suspension, after 24 hours of the suspension sitting un-agitated at ambient temperature (e.g., 25° C.), only a minimal amount of solids will form at the bottom of the container and the remaining suspension should not qualitatively change in either color or appearance. Suspensions that are not stable for predetermined periods will exhibit significant settling, a shift to more reddish color of the suspension, or a change in the opacity of the suspension. FIG. 6 illustrates a comparison of various suspensions prepared from spray dried compositions comprising oltipraz crystals prepared generally according to the method described in Example 1. The spray dried compositions were diluted in preparation for analysis by DLS and then allowed to stand without agitation. As can be seen. Sample D, which was prepared using Dextran 40 as the bulking agent, evidenced significant settling and an increase in the transparency of the suspension, indicating that this particular composition was not stable for a prolonged period. The compositions of the five samples is shown in Table 11 below:

TABLE 11

| Component | Sample A | Sample B | Sample C | Sample D | Sample E |
|---|---|---|---|---|---|
| Oltipraz API | 14.8% | 14.8% | 14.8% | 14.8% | 14.8% |
| Eudragit RL | 7.5% | 7.5% | 7.5% | 7.5% | 7.5% |
| Tween 80 | 3.6% | 3.6% | 3.6% | 3.6% | 3.6% |
| PVP VA64 | 74.0% | | | | |
| Kollidon 30 | | 74.0% | | | |
| Geismar | | | | | |
| Trehalose | | | 74.0% | | |
| Dextran 40 | | | | 74.0% | |
| HPMC-E3 | | | | | 74.0% |

### Example 5: Solubility Analysis of Oltipraz Compositions

The solubility of oltipraz crystals in a spray-dried composition prepared generally according to the method described in Example 1 was measured and compared against the solubility of neat crystalline oltipraz prepared according to the process disclosed in WO2016207914. The MHD of the oltipraz crystals in the spray-dried composition was 369.5 nm, with a polydispersity of 0.324 as measured by DLS after reconstituting the powder in water. The crystals in the neat crystalline oltipraz ranged in size from 20 μm to 200 μm. The solubility was determined at 20° C. both in water and in standard 2% simulated intestinal fluid (Fisher Scientific, USA. Catalog No. 7109-16). The results are reported in Table 12 below.

TABLE 12

| | Measured Solubility |
|---|---|
| Sample Condition | μg/mL (mean n = 2) |
| Neat oltipraz in water | 3.1 |
| Solubility of active ingredient (oltipraz crystals) | 5.7 |
| in spray-dried composition in water | |
| Neat oltipraz in 2% simulated intestinal fluid | 15.8 |
| Solubility of active ingredient (oltipraz crystals) | 22.6 |
| in spray-dried composition in in 2% simulated | |
| intestinal fluid | |

As can be seen, the solubility of the oltipraz crystals in water almost doubled as compared to the neat oltipraz crystals, showing an increase of 83%. The increase in the simulated intestinal fluid was greater than 40%. i.e., approximately 43%.

### Example 6: Lyophilization of Oltipraz Compositions

An 87.5 mg sample containing 50 mg of oltipraz crystals that were nano-milled to less than 300 nm particle size, 25 mg of Eudrogit and 12.5 mg of Tween80 was added to a 5 ml aqueous solution containing 495 mg of PVP-VA64. The sample was frozen with dry ice and subjected to standard lyophilization (freeze drying) on a Labconco lyophilizer at 10×10(−4) mbar vacuum for 4 hours. The resultant powder was compared to powder that was formed by spray drying the same suspension and was found to have substantially the same bulk density and physical characteristics as the sample prepared in Example 1.

### Example 7: MTT Cell Viability and Intracellular ROS Assays Using HGEPp Cells

Accumulation of reactive oxygen species (ROS) coupled with an increase in oxidative stress is implicated in the pathogenesis of many diseases, one of which is mucositis. See, Sonis, A biological approach to mucositis, J Support Oncol 2004; 2:21-36; Halliwell & Whiteman, Measuring reactive species and oxidative damage in vivo and in cell culture: how should you do it and what do the results mean?, British J. Pharmacology, Volume 142, Issue 2, May 2004, 231-255; and Iglesias-Bartolome et al., mTOR Inhibition Prevents Epithelial Stem Cell Senescence and Protects from Radiation-Induced Mucositis, Cell Stem Cell 11, 401-414, Sep. 7, 2012). Free radicals and other reactive species are constantly generated in vivo and cause oxidative damage to biomolecules, a process held in check by multiple antioxidant and repair systems. Recrystallized oltipraz (prepared according to the process disclosed in WO2016207914) and a formulated oltipraz composition prepared generally according to the process described in Example 1 were tested to determine their effect on protecting primary human gingival epithelial cells (HGEPp) cells from oxidative damage induced by hydrogen peroxide (H2O2). Both treatments showed a statistically significant decrease in intracellular Reactive Oxygen Species concentrations in HGEPp cells at 95% confidence level. The formulated oltipraz composition showed a higher protective effect compared to the recrystallized oltipraz at an 80% confidence level. The data showed a numerical increase in the level of protective activity for the formulated oltipraz compositions as compared to the recrystallized oltipraz. The data did reveal a statistically significant decrease in intracellular ROS (P<0.2) for the formulated oltipraz composition as compared to the recrystallized oltipraz.

Objectives

1. measure the effect of recrystallized oltipraz, formulated oltipraz composition and control powder on cell proliferation within HGEPp cells treated with H2O2, using the TACS MTT Cell Viability Assay Kit

2. measure the effect of recrystallized oltipraz and formulated oltipraz on hydroxyl, peroxyl and other reactive oxygen species within HGEPp cells, using Cell Biolabs' OxiSelect™ Intracellular ROS Assay Kit. This assay employs the cell-permeable fluorogenic probe 2′,7′-dichlorodihydrofluorescin diacetate (DCFH-DA) which diffuses into cells and is deacetylated by cellular esterases to a nonfluorescent DCFH which is then rapidly oxidized to highly fluorescent 2′,7′-dichlorodihydrofluorescein (DCF) by ROS.

Materials
Recrystallized oltipraz prepared by Supportive Therapeutics LLC (Appearance: Red Powder (98.6% HPLC purity)
Formulated oltipraz crystals (Supportive Therapeutics LLC), prepared as described above in Example 1. The MHD of the crystals, as measured by dynamic light scattering (DLS), was about 300 nm. (Appearance: Red Powder)
Control Powder (Supportive Therapeutics LLC) prepared as described above in Example 1, but with no oltipraz crystals (Appearance: Red Powder)
HGEPp cells were purchased from CellnTec Advanced Cell Systems AG
TACS MTT Cell Viability Kit was purchased from Trevigen Inc., USA
OxiSelect™ Intracellular ROS Assay Kit was purchased from Cell Biolabs Inc USA.

Methods

Cell Culture

Pooled primary HGEPps were propagated in CnT-Prime epithelial culture medium provided by CellnTec on 100 mm petri dishes coated with 30 mg/ml Type I rat tail collagen (BD Biosciences) diluted in Dulbecco's phosphate-buffered saline (DPBS). This cell type was chosen since the formulated oltipraz compositions described herein have the potential to serve as a treatment for oral mucositis in a suspension formulation, thereby putting such compositions in close contact with HGEPp cells. The cells were harvested when they reached 70-90% confluency as observed by light microscopy. For routine cultivation, the medium was changed every 3 days. For both the cell viability and ROS assays, the cells from passages 3-7 were seeded at 5×10 3, 2.5×10 4, 5×10 4 cells/cm 2 density to grow cell monolayers in 24-well flat-bottomed tissue culture plates and acclimated overnight at 37° C.

Preparation of Dosing Solutions
1. Recrystallized oltipraz was received as a powder from Supportive Therapeutics and a 100 mM DMSO stock was prepared. Further dilutions were prepared in DMSO from the 100 mM DMSO stock and each DMSO dilution was then added into 10 mL of Dulbecco's phosphate-buffered saline to arrive at final concentrations of 10, 50, and 100 μM.
2. The Normal (Control) group contained saline with the same percentage of DMSO as the treated group.
3. All dosing solutions contained 0.3% of DMSO which is well below the maximum tolerated DMSO percent of 0.8% for HGEPp cells.
4. Formulated oltipraz crystals and control powder were received as a powder and a 500 mM DMSO stock solution was prepared for each powder.
5. 5× dilutions were prepared in DMSO from the 500 mM DMSO stock and each DMSO dilution was then added into 10 mL of Dulbecco's phosphate-buffered saline to arrive at final concentrations of 10, 50, and 100 μM of formulated oltipraz composition and control powder.

Cell Survival Assay (TACS MTT Kit)
1. Plate cell concentration was selected to be 6.25×10 5/ml to yield an OD absorbance within the linear portion of the control curve.
2. Once the HGEPp cells were cultured and ready on the microplate, the media was removed from all the wells and discarded. The cells were washed gently with DPBS 2-3 times and the last wash removed and discarded.
3. Added 10 ul of MTT reagent to each well.
4. Incubated the plate for 6 hours at 37° C. Viewed the cells to confirm the appearance of intracellular precipitate using an inverted microscope.
5. Added 100 ul of Detergent Reagent to all wells, including the control wells taking care not to shake the plates
6. Left the plate covered in the dark at room temperature overnight.
7. Removed the plate cover and measured the absorbance of the wells, including the blanks at 570 nm.
8. Determined the average values from triplicate readings after subtracting the average value for the blanks.

Oxidative Stress Measurement ROS Assay (OxiSelect Kit)
1. Prepared and mixed all reagents thoroughly before use. (Kit instruction)
2. Once the HGEPp cells were cultured and ready on a microplate, the media was removed from all the wells and discarded. Washed the cells gently with DPBS 2-3 times. Removed the last wash and discarded it.
3. Added 100 μL of 1×DCFH-DA/media solution to the cells. Incubated at 37° C. for 60 minutes. Removed and discarded the solution.
4. Treated the DCFH-DA loaded cells with recrystallized oltipraz, formulated oltipraz composition and control powder at the targeted concentrations and with saline/DMSO control.
5. Fluorescence was read on a Fluorescence Plate Reader after 1 hour. All treatment media was carefully removed from the wells and discard. The cells were washed 3 times gently with DPBS. Added 100 μL of medium to each well. Added 100 μL of the 2× Cell Lysis Buffer, mixed thoroughly and incubated for 5 minutes. Transferred 150 μL of the mixture to a fresh 24-well plate for fluorescence measurements at 530 nm.

Results

H2O2-Induced Cytotoxicity in a Dose-Dependent Manner

HGEPp cells were exposed to different concentrations of H2O2 for 4 h to examine H2O2-induced oxidative stress. The cells were exposed to 0-0.6 mM H2O2 for 4 h and cell viability was evaluated using the TACS MTT Cell Proliferation Assay Kit. The percentage of cell survival was determined using the ratio of the optical density (OD) of the test sample to the OD of the control×100%. The results showed that H2O2 exposure led to oxidative stress in a concentration-dependent manner. There was 48% reduction in cell number when the cells were treated with 0.3 mM H2O2 (FIG. 7). Therefore, this concentration was taken to be IC50 of H2O2 in HGEPp cells and used in the follow-on experiments. The data is mean+/−SD of 3 experiments in 6 replicate wells.

Effect on H2O2-Induced Oxidative Stress in HGEPp

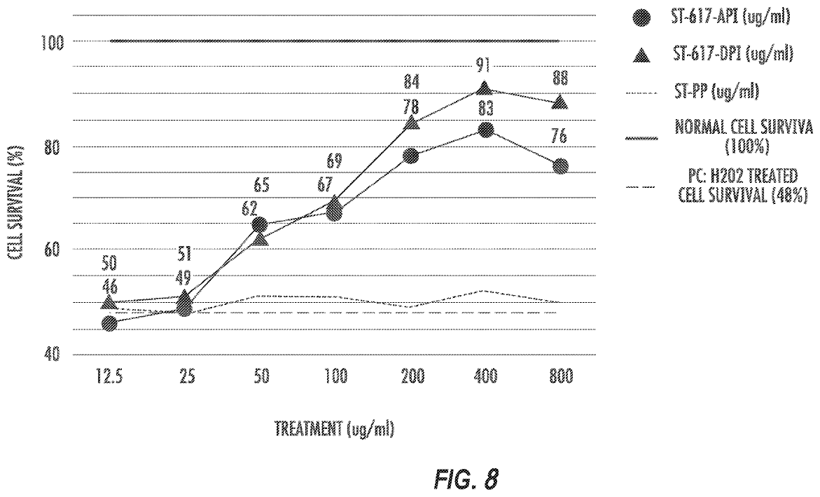

Incubation of HGEPp with H2O2 decreased cell viability significantly (FIG. 7). This viability was modulated by the recrystallized oltipraz and the formulated oltipraz composition, but not by the control powder (FIG. 8). The results indicate that recrystallized oltipraz and formulated oltipraz composition at the concentrations in the range from 50 to 800 μg/ml promoted cell proliferation and reduced H2O2-induced decrease in HGEPp survival.

Normal control cells were cultured in DPBS containing 0.3% DMSO. Positive Control (PC): oxidative stressed group cells after treatment with 0.3 mM H2O2 for 4 h. The remaining groups of cells were pretreated for 24 h with recrystallized oltipraz, formulated oltipraz composition, and the control powder at 12.5, 25, 50, 100, 200, 400.800 μg/mL) prior to treatment with H2O2. The percentage of cell survival was determined by the ratio of the optical density (OD) of the test samples to the OD of the control×100%. The data are presented as the means +/−SD of measurements that were performed in triplicate in six replicate wells, *P<0.05 for recrystallized oltipraz and formulated oltipraz compositions between 50-800 ug/ml versus the PC. The data shows a numerical increase in the level of protective activity for the formulated oltipraz composition as compared to the recrystallized oltipraz.

Effect on ROS Production in HGEPp Cells

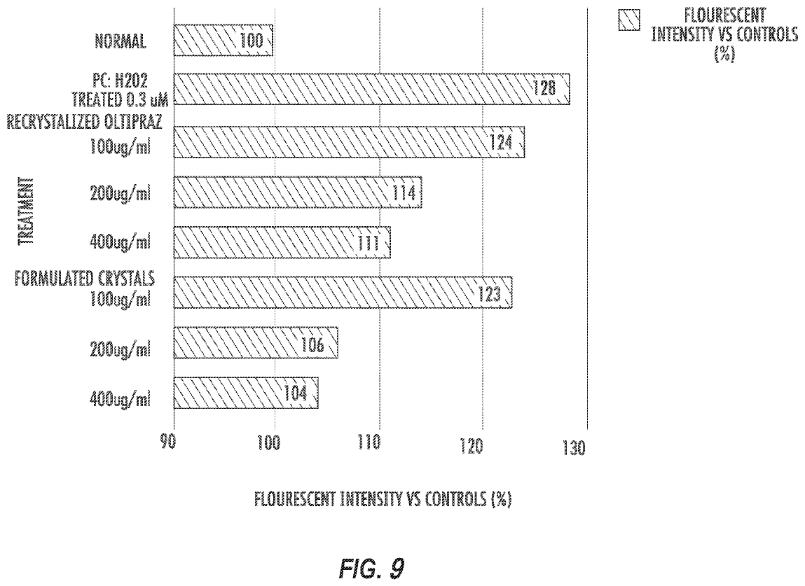

The formation of reactive oxygen species (ROS) is indicative of oxidative stress. There were significantly higher ROS levels (128%) in H2O2-treated hGEP cells compared to normal control cells (100%). The results indicate that recrystallized oltipraz and the formulated oltipraz composition at 100 ug/ml and 200 ug/ml significantly reduced ROS levels in H2O2 treated HGEPp cells. (FIG. 9)

Normal: Normal control cells were cultured in DPBS containing 0.3% DMSO. Positive Control (PC): oxidative stressed group cells after treatment with 0.3 mM H2O2 for 4 h. The remaining groups of cells were pretreated for 24 h with recrystallized oltipraz and formulated oltipraz composition, respectively, at 50, 100, 200 μg/mL prior to treatment with H2O2.

Intracellular ROS was measured using a Spectramax M3 microplate reader. The data are presented as the means +/−SD of measurements that were performed in triplicate in six replicate wells, *P<0.05 for recrystallized oltipraz and formulated oltipraz composition at 100 and 200 ug/ml versus the PC. The data also shows a statistically significant (80%) decrease in intracellular ROS (P<0.2) for the formulated oltipraz composition as compared to the recrystallized oltipraz. That is, the data shows a statistically significant (80% confidence level) superiority for the formulated oltipraz composition as compared to the recrystallized oltipraz.

The pharmaceutical compositions and methods of administering the pharmaceutical compositions of this disclosure thus may be used to treat any human or non-human animal patient to decrease intracellular reactive oxygen species (ROS) and/or decrease oxidative stress, including in patients undergoing treatments that provide oxidative stress such as chemotherapy or radiation therapy. The pharmaceutical compositions and methods of administering the pharmaceutical compositions of this disclosure may be used to treat any human or non-human animal patient to provide an antioxidant effect, including in patients undergoing treatments that provide oxidative stress such as chemotherapy or radiation therapy. The pharmaceutical compositions and methods of administering the pharmaceutical compositions of this disclosure also may be used to slow the onset, and/or reduce the severity, and/or reduce the duration of oxidative damage in patients (e.g., mucositis, including oral mucositis), including in patients undergoing treatments that provide oxidative damage such as chemotherapy or radiation therapy.

### Example 8: Relative Expression of Stress Genes

The Nrf2 system is considered to be a major cellular defense mechanism against oxidative damage by activating genes that encode phase II detoxifying and antioxidant enzymes. The Human oxidative stress PCR array was used to evaluate the relative expression of 84 stress genes after pretreating with 100 uM of recrystallized oltipraz (prepared according to the process disclosed in WO2016207914), formulated oltipraz composition prepared generally in accordance with the process described in Example 1 (MHD less than about 350 nm) and negative control (formulated oltipraz composition without the oltipraz) within HGEPp cells. Total RNA was isolated from treated HGEPp cells, purified and reverse transcription was used to generate cDNA. This was combined with the Qiagen RT2 SYBR Green ROX 96-well array kit and after thermal cycling (BioRad), the gene expressions were recorded (MyiQ detection system) and converted to Fold Change using the Qiagen on-line data analysis tool.

The negative control showed no change in any gene regulation. The recrystallized oltipraz and formulated oltipraz composition both showed up-regulation at Fold Change >2 for ALOX12. GPX1, GCLC. GCLM, NQO1 SOD1 and GAPDH genes and down-regulation at Fold Change >2 for GTF2I, PTGS1 and UCP2 genes.

Only the formulated oltipraz composition additionally showed up-regulation of GPX4 (glutathione peroxidase 4—which is specific to cell membrane antioxidant activity) and MPO (myeloperoxidase) and down-regulation of PRDX2 (Peroxiredoxin 2) at a Fold Change >2.

The pharmaceutical compositions and methods of administering the pharmaceutical compositions of this disclosure thus may be used to treat any human or non-human animal patient to increase the gene expression of GPX4 and/or MPO. The pharmaceutical compositions and methods of administering the pharmaceutical compositions of this disclosure thus also may be used to treat any human or non-human animal patient to decrease the gene expression of PRDX2. The pharmaceutical compositions and methods of administering the pharmaceutical compositions of this disclosure thus also may be used to treat any human or non-human animal patient to increase the gene expression of GPX4 and/or MPO and decrease the gene expression of PRDX2.

### Example 9: Cell Viability and Intracellular ROS Assays Using HGEPP Cells to Determine the Effect of Co-Administering Atovaquone and an Oltipraz Composition

The experiments in this Example were designed to:
1. measure the effect of 12.5-800 μg/mL atovaquone on cell proliferation of HGEPp cells treated with hydrogen peroxide (H2O2), using the TACS MTT Cell Viability Assay Kit.
2. measure any attenuating effect of adding 12.5-800 μg/mL atovaquone with equivalent doses of formulated oltipraz composition prepared generally in accordance with the process described in Example 1 on cell proliferation of HGEPp cells treated with H2O2, using the TACS MTT Cell Viability Assay Kit.
3. measure the effect of 50-200 μg/mL atovaquone on hydroxyl, peroxyl and other reactive oxygen species (ROS) within HGEPp cells, using Cell Biolabs' OxiSelect™ Intracellular ROS Assay Kit.
4. measure any attenuating effect of adding 50-200 μg/mL atovaquone with equivalent doses of formulated oltipraz composition prepared generally in accordance with the process described in Example 1 on hydroxyl, peroxyl and other reactive oxygen species within HGEPp cells, using Cell Biolabs' OxiSelect™ Intracellular ROS Assay Kit. This assay employs the cell-permeable fluorogenic probe 2′,7′-Dichlorodihydrofluorescin diacetate (DCFH-DA) which diffuses into cells and is deacetylated by cellular esterases to a nonfluorescent DCFH which is then rapidly oxidized to highly fluorescent 2′,7′-Dichlorodihydrofluorescein (DCF) by ROS.

Materials
1. Name of Test Article: Formulated oltipraz crystals (Supportive Therapeutics LLC), prepared as described above in Example 1. The MHD of the crystals, as measured by dynamic light scattering (DLS), was about 350 nm. Appearance: Red Powder Batch: 16-00053
2. Name of Test Article: Atovaquone (Sigma Aldrich); Appearance: Yellow powder (>98% purity) Ref. No. A-7986
3. HGEPp cells were purchased from CellnTec Advanced Cell Systems AG
4. TACS MTT Cell Viability Kit was purchased from Trevigen Inc., USA
5. OxiSelect™ Intracellular ROS Assay Kit was purchased from Cell Biolabs Inc. USA.

### Methods

Cell Culture

Pooled primary HGEPp's were propagated in CnT-Prime epithelial culture medium provided by CellnTec on 100 mm petri dishes coated with 30 mg/ml Type I rat tail collagen (BD Biosciences) diluted in Dulbecco's phosphate-buffered saline (DPBS). This cell type was chosen since the formulated oltipraz crystals have the potential to serve as a treatment for oral mucositis in a suspension formulation, thereby putting it in close contact with HGEPp cells. The cells were harvested when they reached 70-90% confluency as observed by light microscopy. For routine cultivation, the medium was changed every 3 days. For both the cell viability and ROS assays, the cells from passages 3-7 were seeded at 5×10 4 cells/cm 2 density to grow cell monolayers in 24-well flat-bottomed tissue culture plates and acclimated overnight at 37° C.

Preparation of Formulated Oltipraz Crystals and Atovaquone Dosing Solutions

Formulated oltipraz crystals were received as a powder from Supportive Therapeutics and a 100 mM DMSO stock solution was prepared from which further dilutions were prepared to arrive at final concentrations of 12.5-800 μg/mL of formulated oltipraz crystals. Atovaquone was received as a powder from Sigma Aldrich and 100 mM DMSO stock solution was prepared from which further dilutions were prepared to arrive at final concentrations of 12.5-800 μg/mL Atovaquone.

Cell Survival Assay (TACS MTT Kit)
1. Plate cell concentration was selected to be 6.25×10 5/ml to yield an OD absorbance within the linear portion of the control curve.
2. Once the HGEPp cells were incubated with test articles for 24 h at 37° C., stressed with 0.3 mM hydrogen peroxide for 4 h at 37° C. the media was removed from all the wells and discarded. The cells were washed gently with DPBS 2-3 times and the last wash removed and discarded.
3. Added 10 μl of MTT reagent to each well.
4. Incubated the plate for 6 hours at 37° C. Viewed the cells to confirm the appearance of intracellular precipitate using an inverted microscope.
5. Added 100 μl of Detergent Reagent to all wells, including the control wells taking care not to shake the plates
6. Left the plate covered in the dark at room temperature for overnight.
7. Removed the plate cover and measured the absorbance of the wells, including the blanks at 570 nm.
8. Determined the average values from triplicate readings after subtracting the average value for the blanks.

Oxidative Stress Measurement ROS Assay (OxiSelect Kit)
1. Prepared and mixed all reagents thoroughly before use. (Kit instruction)
2. Once the HGEPp cells were cultured and ready on a microplate, the media was removed from all the wells and discarded. Washed the cells gently with DPBS 2-3 times. Removed the last wash and discarded it.
3. The cells were treated with saline/DMSO control, formulated oltipraz crystals, and formulated oltipraz crystals+atovaquone at the targeted concentrations and incubated for 24 hrs. The media was removed and cells gently washed with DPBS 2-3 times. Added 0.3 mM H2O2 and incubated at 37° C. for 4 hrs. The media was removed and the cells again washed with DPBS 2-3 times.
4. Added 100 μL of 1×DCFH-DA/media solution to the cells. Incubated at 37° C. for 60 minutes. Removed and discarded the media solution.
5. The cells were washed 3 times gently with DPBS 2-3 times. Added 100 μL of the 2× Cell Lysis Buffer, mixed thoroughly and incubated for 5 minutes. Transferred 150 μL of the mixture to a fresh 24-well plate for fluorescence measurements at 530 nm.

### Results

Effect of Atovaquone, Formulated Oltipraz Crystals and Formulated Oltipraz Crystals+Atovaquone on H2O2-Induced Oxidatively Stressed HGEPp Cells

As seen in Example 7, 0.3 mM H2O2 induces oxidative stress in HGEPp cells within 4 hrs in a dose dependent manner. The same 0.3 mM H2O2 concentration was used in this experiment.

Treatments

Normal: normal control cells were cultured in DPBS containing 0.3% DMSO.

Positive Control (PC): oxidative stressed cells after treatment with 0.3 mM H2O2 for 4 hr.

Atovaquone: group of cells pretreated for 24 hr with atovaquone at 12.5, 25, 50, 100, 200, 400.800 μg/mL prior to treatment with H2O2.

Formulated oltipraz crystals: group of cells pretreated for 24 hr with formulated oltipraz crystals at 12.5, 25, 50, 100, 200, 400, 800 μg/mL prior to treatment with H2O2.

Atovaquone+formulated oltipraz crystals: group of cells pretreated for 24 hr with both atovaquone and formulated oltipraz crystals each at 12.5, 25, 50, 100, 200, 400, 800 μg/mL concentrations prior to treatment with H2O2.

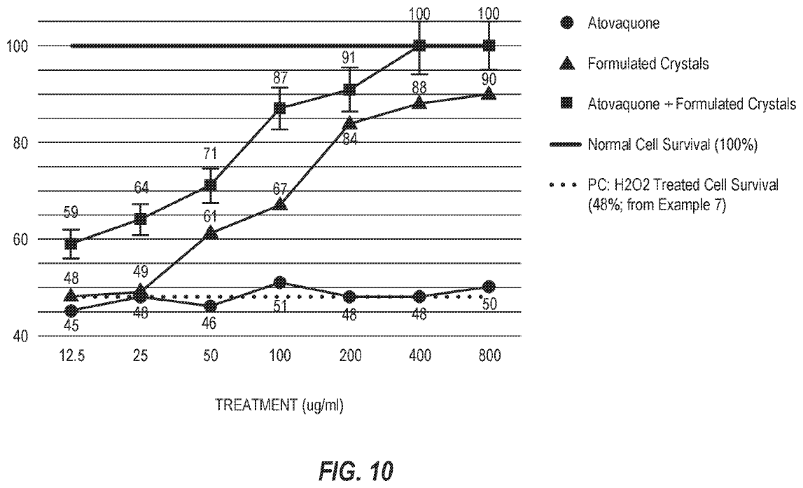

As seen in FIG. 10, atovaquone showed no impact on H22-induced oxidatively stressed HGEPp cell proliferation at any dose tested. (12.5-800 μg/mL). Formulated oltipraz crystals at 50-800 μg/mL doses promoted cell proliferation and increased cell survival in H2O2-induced oxidatively stressed HGEPp cells as seen in Example 7. Atovaquone+formulated oltipraz crystals at all doses tested, 12.5 to 800 μg/mL, significantly increased cell proliferation/survival in H2O2-induced oxidatively stressed HGEPp cells, over treatment with formulated oltipraz crystals alone. The percentage of cell survival was determined by the ratio of the optical density (OD) of the test samples to the OD of the control×100%. The data are presented as the means+/−SD of measurements that were performed in triplicate in six replicate wells. *P<0.05 for atovaquone, formulated oltipraz crystals and atovaquone+formulated oltipraz crystals.

No reduction in the protective effect of formulated oltipraz crystals above 400 μg/ml was observed in this trial.

The Effect of Atovaquone, Formulated Oltipraz Crystals and Formulated Oltipraz Crystals+Atovaquone on ROS Production in H2O2-Induced Oxidatively Stressed HGEPp Cells

The formation of reactive oxygen species (ROS) is indicative of oxidative stress. As seen in Example 7, there were significantly higher ROS levels (133%) in H2O2-treated hGEP cells compared to normal control cells (100%).

Treatments

Normal: normal control cells were cultured in DPBS containing 0.3% DMSO.

Positive Control (PC): oxidatively stressed cells after treatment with 0.3 mM H2O2 for 4 hr.

Atovaquone: cells were pretreated for 24 hr with atovaquone at 50, 100, 200 μg/mL prior to treatment with H2O2.

Formulated oltipraz crystals: group of cells pretreated for 24 hr with formulated oltipraz crystals at 50, 100, 200 μg/mL prior to treatment with H2O2.

Atovaquone+formulated oltipraz crystals: group of cells pretreated for 24 hr with both atovaquone and formulated oltipraz crystals each at 50, 100.200 μg/mL concentrations prior to treatment with H2O2.

Intracellular ROS was measured using a Spectramax M3 microplate reader. The data are presented as the means+/−SD of measurements that were performed in triplicate in six replicate wells.

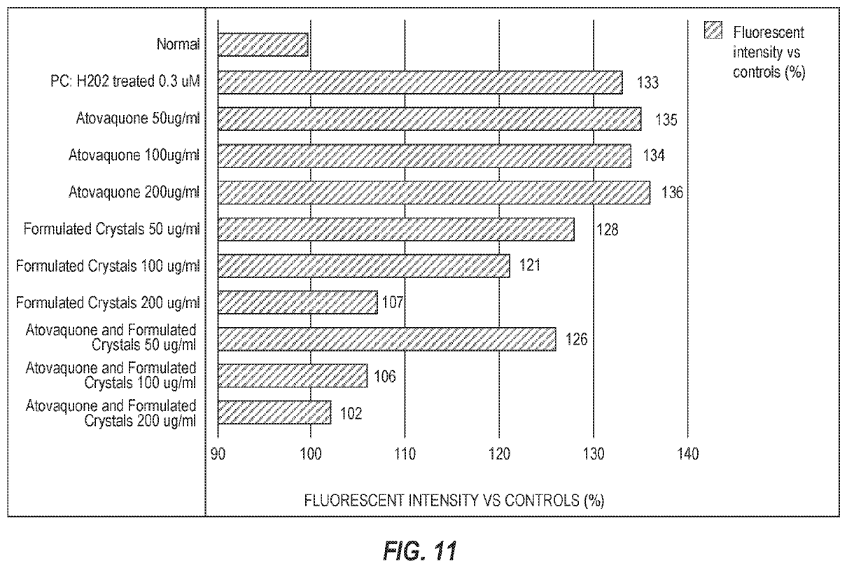

FIG. 11 shows that atovaquone by itself does not change ROS levels in H2O2 challenged HGEPp cells. However, atovaquone at the 100 μg/mL co-administered dose with formulated oltipraz crystals significantly reduced ROS levels (*P<0.05), when compared to the effect of formulated oltipraz crystals alone at the same dose. These results are consistent with those in example 7, where formulated oltipraz crystals alone at 100 and 200 y g/mL dose showed ROS levels of 123% and 106% as compared to 121% and 107% in this assay.

### Example 10: Cell Viability and Intracellular ROS Assays Using HGEPP Cells to Determine the Effect of Co-Administering Metformin and an Oltipraz Composition

The experimental procedure of Example 9 is repeated, but the atovaquone concentrations are replaced with an equivalent concentration of metformin. Metformin is a drug used by patients with type 2 diabetes to, among other things, help restore the patient's proper response to insulin that the patient's body naturally produces, and is available from Sigma Aldrich. The metformin shows little or no impact on H2O2-induced oxidatively stressed HGEPp cell proliferation at any dose tested. (12.5-800 μpg/mL). Formulated oltipraz crystals at 50-800 μg/mL doses promote cell proliferation and increased cell survival in H2O2-induced oxidatively stressed HGEPp cells as seen in Example 7. Metformin+formulated oltipraz crystals at all doses tested, 12.5 to 800 μg/mL, will increase cell proliferation/survival in H22-induced oxidatively stressed HGEPp cells, over treatment with formulated oltipraz crystals alone. The percentage of cell survival may be determined by the ratio of the optical density (OD) of the test samples to the OD of the control×100%.

### Example 11: Cell Viability and Intracellular ROS Assays Using HGEPP Cells to Determine the Effect of Co-Administering Berberine and an Oltipraz Composition

The experimental procedure of Example 9 is repeated, but the atovaquone concentrations are replaced with an equivalent concentration of berberine, which is a supplement that is used by some people to promote healthy blood sugar levels. The berberine shows little or no impact on H2O2-induced oxidatively stressed HGEPp cell proliferation at any dose tested. (12.5-800 μg/mL). Formulated oltipraz crystals at 50-800 μg/mL doses promote cell proliferation and increased cell survival in H2O2-induced oxidatively stressed HGEPp cells as seen in Example 7. Berberine+formulated oltipraz crystals at all doses tested, 12.5 to 800 μg/mL, will increase cell proliferation/survival in 11202-induced oxidatively stressed HGEPp cells, over treatment with formulated oltipraz crystals alone. The percentage of cell survival may be determined by the ratio of the optical density (OD) of the test samples to the OD of the control×100%.

### Example 12: Prevention and Treatment of Reperfusion Injury Following Organ Transplantation Surgery by Co-Administering Atovaquone and an Oltipraz Composition

A patient who is scheduled to undergo organ transplantation receives atovaquone (e.g., 750 mg twice a day with food or 1500 mg once per day with food) for one, two or three days in advance of the surgery. Prior to the surgery, the patient also receives one or more doses comprising oltipraz, either in recrystallized form or in a formulation as described in Example 9, which dose(s) contains an amount of oltipraz that is within the range of 125 mg to 4.5 g or more of oltipraz. Following transplantation surgery, the patient experiences less reperfusion injury than she would have in the absence of the co-administration of atovaquone and oltipraz.

Optionally, a composition that comprises an OCR-API such as atovaquone can be used for storage, transport and/or reperfusion of the organ. Optionally, in addition to or instead of the OCR-API, the composition may comprise oltipraz and/or another Nrf2 activator(s) such as sulphoraphane, phenethyl isothiocyanate, curcumin, resveratrol, fumaric acid and its esters, and synthetic oleanane triterpenoids.

### Example 13: Prevention and Treatment of Reperfusion Injury Following Organ Transplantation Surgery by Co-Administering Metformin and an Oltipraz Composition

A patient who is scheduled to undergo organ transplantation receives Metformin in an amount of between 500 mg and 2550 mg each day for one, two or three days prior to surgery. Prior to the surgery, the patient also receives one or more doses comprising oltipraz, either in recrystallized form or in a formulation as described in Example 9, which dose(s) contain an amount of oltipraz that is within the range of 125 mg to 4.5 g or more of oltipraz. Following transplantation surgery, the patient experiences less reperfusion injury than she would have in the absence of the co-administration of metformin and oltipraz.

Optionally, a composition that comprises an OCR-API such as metformin can be used for storage, transport and/or reperfusion of the organ. Optionally, in addition to or instead of the OCR-API, the composition may comprise oltipraz and/or another Nrf2 activator(s) such as sulphoraphane, phenethyl isothiocyanate, curcumin, resveratrol, fumaric acid and its esters, and synthetic oleanane triterpenoids.

### Example 14: Prevention and Treatment of Reperfusion Injury Following Organ Transplantation Surgery by Co-Administering Berberine and an Oltipraz Composition

A patient who is scheduled to undergo organ transplantation receives berberine in an amount of between 500 mg-1500 mg each day for one, two or three days prior to surgery. Prior to the surgery, the patient also receives one or more doses comprising oltipraz, either in recrystallized form or in a formulation as described in Example 9, which dose(s) contain an amount of oltipraz that is within the range of 125 mg to 4.5 g or more of oltipraz. Following transplantation surgery, the patient experiences less reperfusion injury than she would have in the absence of the co-administration of berberine and oltipraz.

Optionally, a composition that comprises an OCR-API such as berberine can be used for storage, transport and/or reperfusion of the organ. Optionally, in addition to or instead of the OCR-API, the composition may comprise oltipraz or one or more other Nrf2 activator(s) such as sulphoraphane, phenethyl isothiocyanate, curcumin, resveratrol, fumaric acid and its esters, and synthetic oleanane triterpenoids.

### Example 15: Prevention and Treatment of Reperfusion Injury Following Myocardial Infarction by Co-Administering Atovaquone and an Oltipraz Composition

A patient who is experiencing myocardial infarction is administered atovaquone (750 mg-1500 mg). At the same time or shortly before or shortly after, the patient is administered a dose comprising oltipraz, either in recrystallized form or in a formulation as described in Example 9, which dose contains an amount of oltipraz that is within the range of 125 mg to 4.5 g or more of oltipraz. Following a medical procedure that improves the blood flow to the patient's heart, the patient's heart experiences less reperfusion injury than he would have in the absence of the co-administration of atovaquone and oltipraz.

Optionally, the patient is administered one or more additional Nrf2 activators such as sulphoraphane, phenethyl isothiocyanate, curcumin, resveratrol, fumaric acid and its esters, and synthetic oleanane triterpenoids in addition to, or instead of, the oltipraz.

### Example 16: Prevention and Treatment of Reperfusion Injury Following Myocardial Infarction by Co-Administering Metformin and an Oltipraz Composition

A patient who is experiencing myocardial infarction is administered a dose of Metformin in an amount of between 500 mg and 2550 mg. At the same time, or shortly before or shortly after, the patient also is administered a dose comprising oltipraz, either in recrystallized form or in a formulation as described in Example 9, which dose contains an amount of oltipraz that is within the range of 125 mg to 4.5 g or more of oltipraz. Following a medical procedure that improves the blood flow to the patient's heart, the patient's heart experiences less reperfusion injury than he would have in the absence of the co-administration of atovaquone and oltipraz.

Optionally, the patient is administered one or more additional Nrf2 activators such as sulphoraphane, phenethyl isothiocyanate. curcumin. resveratrol, fumaric acid and its esters, and synthetic oleanane triterpenoids in addition to, or instead of, the oltipraz.

### Example 17: Prevention and Treatment of Reperfusion Injury Following Myocardial Infarction by Co-Administering Berberine and an Oltipraz Composition

A patient who is experiencing myocardial infarction is administered a dose of herberine (500 mg-1500 mg). At the same time, or shortly before or shortly after, the patient is administered a dose comprising oltipraz, either in recrystallized form or in a formulation as described in Example 9, which dose contains an amount of oltipraz that is within the range of 125 mg to 4.5 g or more of oltipraz. Following a medical procedure that improves the blood flow to the patient's heart, the patient's heart experiences less reperfusion injury than he would have in the absence of the co-administration of atovaquone and oltipraz.

Optionally, the patient is administered one or more additional Nrf2 activators such as sulphoraphane, phenethyl isothiocyanate, curcumin, resveratrol, fumaric acid and its esters, and synthetic oleanane triterpenoids in addition to, or instead of, the oltipraz.

### Example 18: Protective Effects of Oltipraz and Oltipraz and Atovaquone in Preventing Neurotoxicity Induced by Oxygen Glucose Deprivation

Stroke is a severe and devastating neurological disease globally and is the leading cause of permanent disability in adults. Ischemic stroke is characterized by apoptotic and necrotic cell death leading to neuronal loss which lead to both rapid and delayed injury to brain parenchyma. To date, there is no effective neuroprotective drug in clinical use, therefore there is an urgent need to develop new therapies of neuroprotection against stroke injury. This Example explored the neuroprotective signaling effect of four compounds in an established in vitro model of CNS injury induced by oxygen-glucose depletion.

The objective of the experiments in this Example was to explore the protective effects of three compounds in the neurotoxicity induced by oxygen glucose deprivation. A multiparametric cell-based protocol is used in this Example in order to determine the mechanisms of action. The assay was applied to “freshly isolated” rat cortical neurons cultured in 96-well-plates and exposed to the compounds at 100 μg/ml. Moreover, some cells were treated with the compounds in addition with atovaquone (100 μg/ml).

### Overall Methods

In vitro ischemic injury was induced in rat primary neuronal culture by oxygen-glucose deprivation for 2 hours. Neurons were incubated with the compounds during 1 hour eighteen hours prior to the OGD induction. MK801 10 μM was used as positive neuroprotection control. After the treatments, supernatants were collected for measuring extracellular LDH levels (plasma membrane integrity determination) and cells were simultaneously incubated with three optically compatible fluorescent dyes, and subsequently analyzed with BD Pathway 855 (Becton Dickinson). Cell parameters associated with nuclear morphology, mitochondrial damage and caspase 3/7 activation-indicative of prelethal cytotoxic effects and representative of different mechanism of toxicity-were measured by HCS at single cell level. Finally, cells were fixed and stained with beta III tubulin antibody for measuring the neurite outgrowth.

Attending to the data obtained, the compounds were classified in four groups according to their degree of neuroprotection: high, moderate, low and no neuroprotection.

The protocol, which utilizes cortical neurons from embryonic 18 days rats, was introduced to screen the neuroprotective action of peptides by measuring lactato deshidrogenase, caspase 3/7 activation, mitochondrial damage, cell counting, neurite outgrowth, lactato dehidrogenase release of each compounds:
Formulated oltipraz crystals (“DPI”) (100 μg/ml),
Formulated oltipraz crystals (“DPI”)+atovaquone (100 μg/ml+100 μg/ml).
Recrystallized oltipraz (“API”)+atovaquone (100 μg/ml+100 μg/ml)
MK801—a neuroprotective positive control (dizocipine)

TABLE 13
Table of Abbreviations

| ABBRE- | DESCRIP- | ABBRE- | DESCRIP- |
|---|---|---|---|
| VIATION | TION | VIATION | TION |
| nm | Nanometers | FBS | Fetal Bovine Seram |
| nM | Nanomolar | DMSO | Dimethyl sulfoxide |
| mM | Millimolar | OGD | Oxygen glucose |
| | | | deprivation-hypoxia |
| SD | Standard | TMRM | tetramethyl rhodamine |
| | Deviation | | methyl ester |
| Ato | Atovaquone | CNS | Central Nervous system |

The following abbreviations are used in this Example:

### Materials and Methods

TABLE 14
Reagents and Equipment

| Reagent/Equipment and Catalogue | |
|---|---|
| and lot Numbers | Supplier |
| Neurobasal (# Cat, 21103-049) | Invitrogen |
| B-27 (# Cat. 17504-044) | Invitrogen |
| FBS (# Cat. F7524) | Sigma Aldrich |
| Trypsin-EDTA 0.5% (w/v) (# Cat. 25300) | Gibco |
| DMSO (# Cat. D2650) | Sigma Aldrich |
| Penicillin/Streptomycin (# Cat. 15240) | Gibco |
| Centrifuge (# Allegra X-22R) | Beckman Coulter |
| Incubator (# Model 381 S/N 314342) | Thermo Scientific |
| PBS (# Cat. D8537) | Sigma Aldrich |
| Laminar flow cabinet (# ESCO class | Lab Culture |
| II BSC) | |
| Trypan blue 0.4% (# Cat. TI0282) | Invitrogen |
| Poli-L-lysine (# Cat. P1524) | Sigma Aldrich |
| Dnase I (# Cat. D5025) | Sigma Aldrich |
| Trypsin (# CatT4799) | Sigma Aldrich |
| Pathway 855 (no A107) | Becton Dickinson |
| LDH kit (# Cat 11644793001) | Roche |
| TMRM dye (# Cat. 88065) | Anaspec Inc. |
| Cell event Caspase 3/7 green detection | Invitrogen |
| (# Cat. C10423) | |
| Anti-beta III tubulin (ab7751) | Abcam |
| Anti goat-antimouse alexa 488 (# Cat. | Abcam |
| A21050) | |
| HOECHST (# Cat. H1399) | Invitrogen |
| Hypoxia chamber (# Cat. 5352414) | Billups-Rothenberg |
| Synergy II microplate reader | Biotek Instruments |
| Cell counting Kit-8 (# Cat. 96992) | Sigma-Aldrich |
| NGF (#Cat. 13257-019) | Invitrogen |
| Neurobasal without glucose (# Cat. | Invitrogen |
| A24775) | |

Test Compounds
“API”—Recrystallized oltipraz (Appearance: Red Powder (98.6% HPLC purity; weight 1 g)
“DPI”—Formulated oltipraz crystals prepared as described above in Example 1. The MHD of the crystals, as measured by dynamic light scattering (DLS), was about 300 nm. (Appearance: Red Powder; weight 1 g)
Atovaquone—Sigma Aldrich, weight 10 mg (solubilized in DMSO)

### Isolation and Culture of Neurons.

Primary cultures of cortical neurons were isolated from cerebral cortices of Sprague-Dawley rat foetuses at embryonic day 18. Brains were removed and after removing the meninges, the tissues were dissected under a binocular microscope. Neurons were enzymatically dispersed by trypsin 0.2% and DNAse 10.04% digestion for 10 min at 37° C., plated in poly-L-lysine wells and incubated with B27 supplemented neurobasal medium.

### Experimental Assay of Toxicity Induced by Oxygen Glucose Deprivation.

Experimental Procedure.

Cortical neurons of 18 days old embryonic rats were plated in poly-l-lysine coated 96-well plates (30.000 cells per well). Cells were maintained in neurobasal medium supplemented with B-27 component for 5 days at 37° C. in a humidified 5% CO2 atmosphere. At day 5, cells were subjected to oxygen glucose deprivation (OGD) for 2 hours at 37° C. The cultures were placed in an aerobic chamber (Billops-Rothenberg) and incubated in neurobasal medium lacking glucose and B27 factor, and aerated with an anaerobic gas mixture (94.7% N2, 5% CO2, 0.3% O2) to remove residual oxygen. Control cultures were kept in the original neurobasal medium but were submitted to the anaerobic conditions. At the end of the OGD conditions, the cells were removed from the anaerobic chamber, the OGD medium was replaced with neurobasal medium containing glucose, and the cells were incubated for an additional 24 h. The test compounds were added to the neurons eighteen hours prior to the OGD induction and maintained for 1 h. Replicate cultures were treated as described above with MK801 as positive control of neuroprotection.

HCS Assay: Incubation and Imaging of Fluorescent Probes and Beta III Tubulin Immunostaining.

Compound neuroprotective potential was determined by HCS analysis, which included the following endpoints: plasma membrane integrity, cell viability, alterations of mitochondrial membrane potential, caspase 3/7 activation and neurite outgrowth.

LDH Assay:

To determine the integrity of the plasma membrane, supernatants were collected 24 h after treatments and LDH assay was performed following manufacturer's instructions.

Cell Number:

Cell number was determined by hoechst 33342 nucleic acid staining. This dye allows a sensitive cell number determination by fluorescence microscopy. Cells were stained with 5 μg/ml, washed 3 times and measured at 380 nm/460 nm Ex/Em.

Mitochondrial Damage:

Mitochondrial activity was determined using TMRM, a lipophilic cationic fluorescent probe that freely crosses the plasma membrane and accumulates within mitochondria, depending on their membrane potential. Cells were stained with 50 nM tetramethyl rhodamine methyl ester-TMRM, washed 3 times and measured at 555 nm/645 nm Ex/Em. TMRM fluorescence intensity in cytosolic regions around the nucleus was registered.

Caspase 3/7 Activation:

Caspase 3/7 activation was determined using The CellEvent® Caspase-3/7 Green Detection Reagent which is intrinsically a non-fluorescent peptide that inhibits the ability of the dye to bind to DNA. However, after caspase-3/7 activation in apoptotic cells, the peptide is cleaved allowing the dye to bind to DNA producing a bright, fluorogenic response. Cell were stained with 5 μM reagent, washed 3 times and measured at 488 nm/530 nm Ex/Em. This dye permits the direct quantification of apoptotic cells.

Cell Viability

For detection of viable cells, 10 μl of CCK-8 reagent (WST-8) were added to each well and the plate was incubated at 37° C. After 1 hour, absorbance was measured at 450 nm using the Synergy II microplate reader.

Beta-III Tubulin Staining:

Beta-III tubulin staining was determined by IHQ. Once the cells were stained and imaged with fluorescent dyes, cells were washed with PBS and fixed with 4% paraformaldehyde for 15 minutes. After the fixation step, the samples were washed three times with PBS and permeabilized with PBS+0.3% triton for 10 minutes. The samples were then blocked with PBS+Bovine Serum Albumine (BSA) for 30 minutes and finally anti-tubulin III antibody was added at 1/1000 in PBS+0.5% BSA for 60 minutes at room temperature. After three washing steps, the secondary antibody Alexa 633 was added at 1/100 for 60 minutes. The samples were then washed three times and measured in the Pathway 855 automated fluorescent microscope. To investigate the role of neurite extension, a geometric pattern was mainly employed in this study: average length per neuron.

At the end of the assay, supernatants were collected for performing LDH assay and cells were simultaneously loaded with fluorescent dyes. After 1 h of incubation at 37° C. with the culture media containing the probes, cells were imaged using the 20× objective in the BD Pathway 855, and the Attovision software. Then, cells were fixed for beta-M tubulin staining and imaged again. To acquire enough data for the analysis, nine fields per well were imaged. During the set-up of the procedure, the exposure time was adjusted to avoid overlapping emission between the different probes. The collected images were further analyzed using a module that allows simultaneous quantification of subcellular structures, which are stained by different molecular probes and that measures the fluorescence intensity associated with predefined nuclear and cytoplasmic compartments.

Analysis of HCS Data.

For all the compounds and parameters studied, a variation of at least 20% in fluorescence intensity or in the corresponding morphological parameter in relation to untreated cultures was considered as significant. In order to compare their neuroprotective potential, variations for each parameter after 24 h were studied at each concentration. The following neuroprotection criteria was established according to the level of variation when compared with control cells: 0 (no neuroprotection or variation lower than 20%), 1 (variation 20-40%), 2 (variation 40-60%), 3 (variation 60-100%) and 4 (variation >100%). In some cases, the incubation of OGD plus compounds caused toxicity (extra LDH increase) and an injury scale was also stablished: −1 (variation 20-40%), −2 (variation 40-60%). The sum of each individual score resulted in the total level of neuroprotection for each compound and was defined as its degree of neuroprotection. From this calculation, a neuroprotective scale was established: high (>8), moderate (5-7), low (1-4) and no neuroprotection (0).

### Results

Neuroprotection of Compounds Against OGD Toxicity

Cell Number.

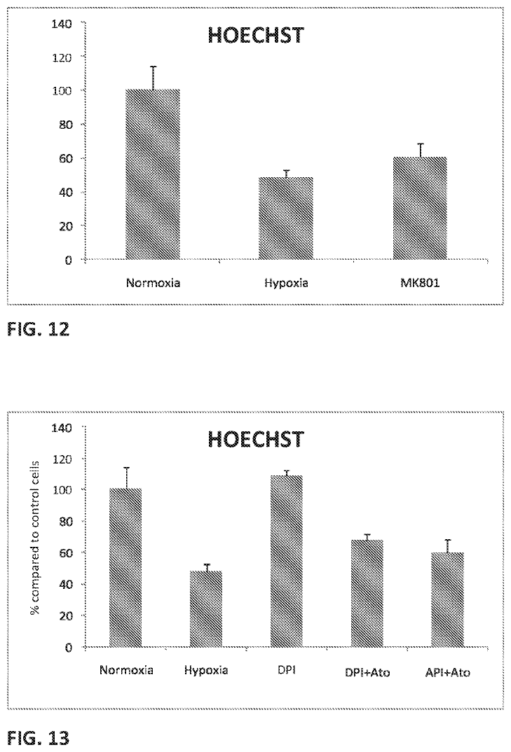

OGD treatment resulted in 52% of mortality in neurons when measured 24 h after exposure. On the other hand, the known neuroprotective molecule MK801, prevents OGD-induced mortality by 26% (FIG. 12). OGD-induced cell death could be also prevented by application of the three compounds. Therefore, compounds supplementation significantly reduced cell death and improved survival of neurons. Different grades of neuroprotection were observed with each compound at the different concentrations tested (FIG. 13).

Apoptosis

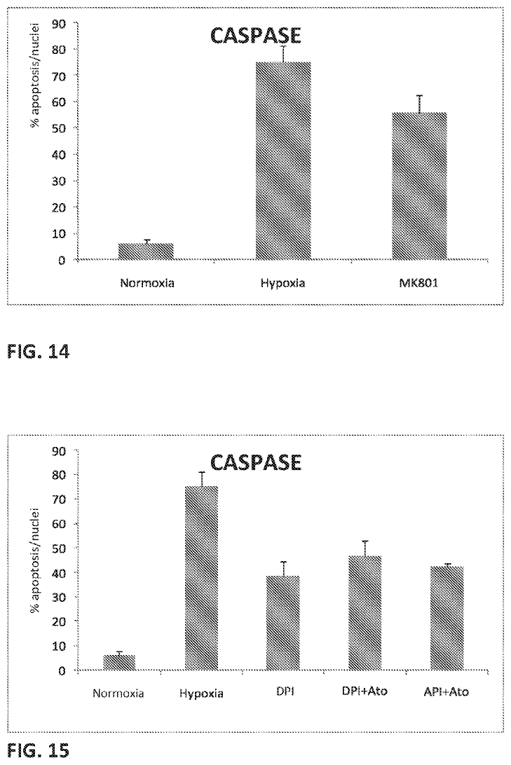

OGD treatment resulted in an increase of 12 fold of caspase 3/7 activation in neurons when measured 24 h after exposure whereas the neuroprotection positive control MK801, prevents OGD-induced apoptosis by 25% (FIG. 14). OGD induced caspase 3/7 activation could be also prevented by the application of all compounds tested. Therefore, compounds supplementation significantly reduced caspase 3/7 activation and improved survival of neurons. Different grades of neuroprotection were observed with compounds at the different concentrations tested (FIG. 15).

Neurite Outgrowth

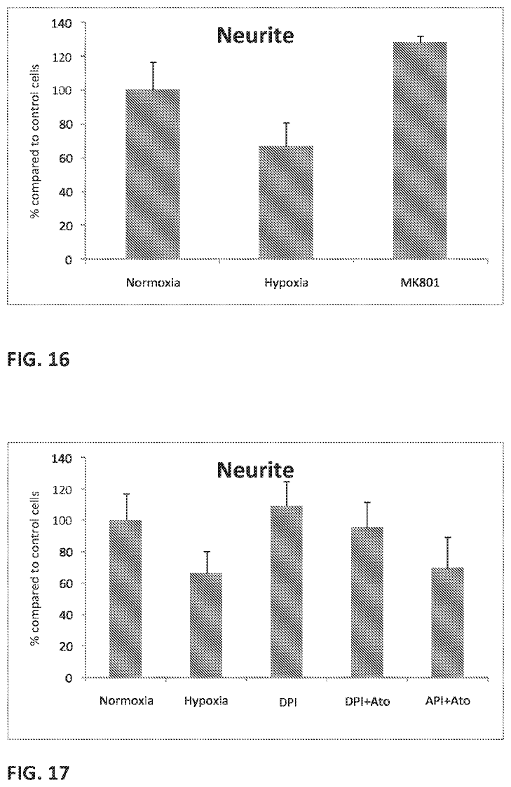

To investigate the role of neurite extension one geometric pattern was employed in this study: average length. OGD treatment resulted in a neurite outgrowth decrease of 34% compared to untreated cells, and on the other hand, positive control MK801 could re-establish by 94% the affected neurite outgrowth induced by OGD (FIG. 16). OGD-induced neurite outgrowth reduction could be prevented by application of the compounds DPI and DPI+Ato (FIG. 17).

Plasma Membrane Integrity.

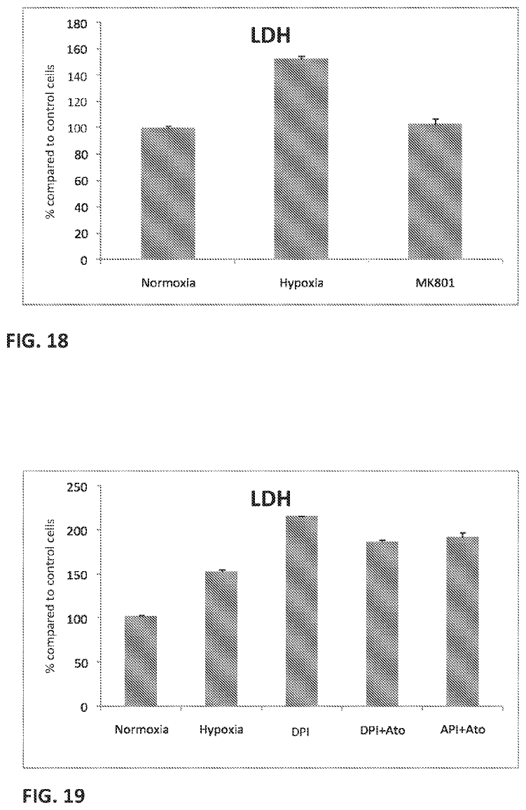

To assess the effects of OGD on plasma membrane integrity, LDH quantification in supernatants of treated cells was employed. It was observed that OGD caused an increase of LDH release of 52% in cultured cortical neurons, while positive control MK801 could normalize plasma membrane integrity by 33% (FIG. 18). OGD induced cell death could not be prevented by application of any of the compounds alone or in combination with compound 1. It was observed that the three compounds in addition to OGD induced an increase in LDH production (FIG. 19).

Mitochondrial Damage.

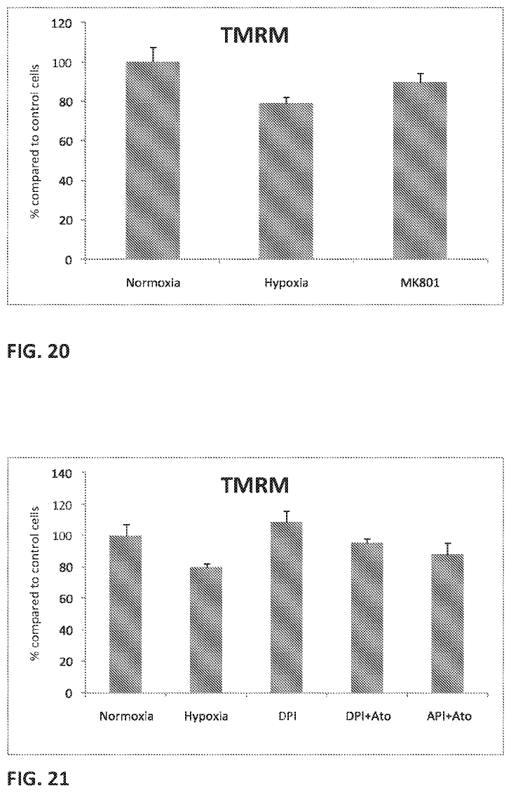

Some studies have shown that OGD affects mitochondrial membrane depolarization/hyperpolarization and to assess the effects of OGD on mitochondrial membrane potential. TMRM dye was employed. The results revealed polarized normal cells in control cells whereas OGD treatment showed a 20% of population with depolarized cells at 24 hours post-exposure. MK801 compound could only re-establish mitochondrial membrane potential by 13% (FIG. 20). OGD-induced depolarized reduction could be increased by application of the compounds DPI and DPI+Ato (FIG. 21).

Cell Viability

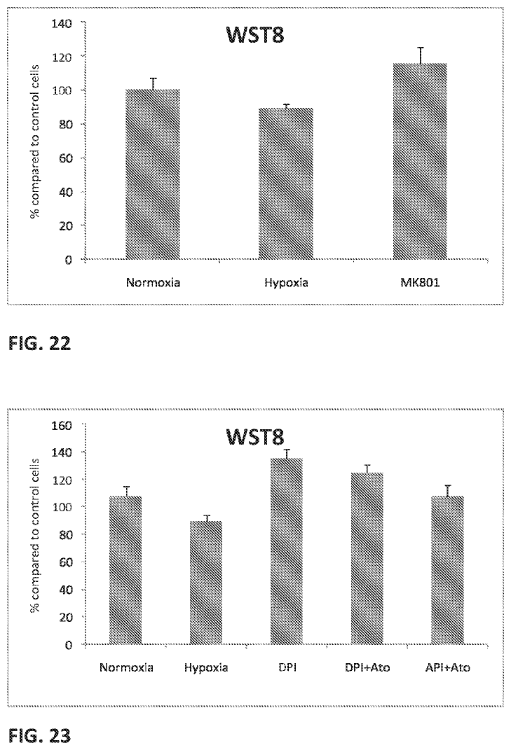

Glutamate treatment resulted in a decrease of 15% of cell viability measured by WST-8 in neurons when measured 24 h after exposure to OGD conditions. Therefore, the effect size was too small to make conclusions on compound effects with the WST-8 assay but a tendency of compounds to recover OGD-induced mitochondrial damage was observed (FIGS. 22 and 23).

In order to compare the degree of neuroprotection of tested compounds, different scores were created according to the level of the alteration (the group formation criterion is described in material and methods section) and then a value was assigned to each parameter. The sum of all the values for each parameter and concentration tested was calculated, and four distinct groups were created according to the level of neuroprotection provided. From this analysis, the degree of neuroprotection of each compound studied was defined (Table 15).

Briefly, the formulated oltipraz crystals (DPI), with or without atovaquone, scored moderate and high neuroprotection level respectively. Recrystallized oltipraz (API) with atovaquone provided a lower neuroprotection level. The positive control as MK801 scored moderate neuroprotection (Table 16).

TABLE 15

| | DPI | DPI + ATO | API + ATO |
|---|---|---|---|
| HOECHST | 4 | 2 | 1 |
| CASPASE | 2 | 1 | 2 |
| Neurite | 3 | 2 | 0 |
| TMRM 1 | 1 | 1 | 0 |
| LDH | −1 | −1 | −1 |
| Total | 9 | 5 | 2 |
| Neuroprotection degree | high | moderate | low |

TABLE 16

| | MK801 |
|---|---|
| HOECHST | 1 |
| CASPASE | 1 |
| Neurite | 3 |
| TMRM | 0 |
| LDH | 1 |
| Total | 6 |
| Neuroprotection degree | moderate |

Conclusion

In this Example, OGD toxicity is linked to an increase in caspase 3/7 activation, LDH secretion and mitochondrial potential. On the other hand, a decrease in cell number, mitochondrial potential and neurite outgrowth is also present. The preventive effects shown by some compounds against OGD toxicity are associated with increase of cell viability, restoration of caspase 3/7 activity, stabilization of neurite outgrowth and mitochondrial potential, and a decrease of LDH secretion.

The formulated oltipraz crystals with or without atovaquone scored high and moderate neuroprotection level respectively. Based on the data, these compositions appear to be an efficient strategy for the treatment of OGD-induced toxicity. Recrystallized oltipraz with atovaquone provided neuroprotection, but scored a lower neuroprotection level.

It is noted that the compounds separately dissolved correctly but when preparing the formulated oltipraz crystals plus atovaquone, and recrystallized oltipraz plus atovaquone, some red precipitates were observed. Precipitation was higher in the case of recrystallized oltipraz plus atovaquone solution and the precipitates could not be completely dissolved by heat and sonication. Accordingly, the actual level of neuroprotection provided by the formulated oltipraz crystals and recrystallized oltipraz through the addition of atovaquone, if completely dissolved through appropriate means, could be greater than that observed. Moreover, atovaquone is more readily absorbed with meals, as is oltipraz, and thus absorption is enhanced by lipids, e.g., vegetable oil, which may be reflected in the poor solubility as well.

### Example 19: Protective Effects of Oltipraz and Oltipraz and Atovaquone in Preventing Cardiotoxicity Induced by Oxygen Glucose Deprivation

Example 19 was designed to explore the protective effects of three compounds against the cardiotoxicity induced by oxygen-glucose deprivation using a multiparametric cell-based protocol that suggests their mechanisms of action. The assay was applied to “freshly isolated” mouse cardiomyocytes cultured in 96-well-plates and exposed to the compounds at 100 μg/ml. Moreover, some cells were treated with the compounds and in addition with atovaquone (100 μg/ml).

Heart ischemia is the leading cause of death worldwide, and it is the second most common cause of emergency department visits in the World. Ischemia is a condition where the flow of oxygen-rich blood to a part of the body is restricted. Cardiac ischemia refers to lack of blood flow and oxygen to the heart muscle. Cardiac ischemia happens when an artery becomes narrowed or blocked for a short time, preventing oxygen-rich blood from reaching the heart. If ischemia is severe or lasts too long, it can cause a heart attack (myocardial infarction) and can lead to heart tissue death.

Ischemic stroke is characterized by apoptotic and necrotic cell death leading to cardiomyocyte loss, which lead to both rapid and delayed injury to cardiac parenchyma. To date, there is no effective cardioprotective drug in clinical use, therefore there is an urgent need to develop new therapies of cardioprotection against heart ischemia.

In Example 18, the experiments characterized the cardioprotective signaling pathway of three compounds in an established in vitro model of cardiac injury induced by oxygen-glucose depletion. The protocol that utilizes cardiomyocytes from neonatal 0-2 days mice was introduced to screen the cardioprotective action of compounds by measuring caspase 37 activation, cell counting, ATP intracellular measure and LDH release of each compounds at various different concentrations.
Formulated oltipraz crystals (“DPI”) (100 μg/ml),
Formulated oltipraz crystals (“DPI”)+atovaquone (100 μg/ml+100 μg/ml),
Recrystallized oltipraz (“AP”)+atovaquone (100 μg/ml+100 μg/ml)

### Methods

In vitro ischemic injury was induced in mouse neonatal primary cardiomyocyte culture by oxygen-glucose deprivation for 18 hours. Cardiomyocytes were incubated with two concentrations of the compounds during 4 hours prior to the OGD induction. The test compounds were also present during the OGD insult and 24 h-recovery period. NAC was used as positive control of cardioprotection. After treatments, cell viability was measured with WST-8 and ATP, and supernatants were collected to measure LDH for studying plasma membrane integrity. Cells were simultaneously loaded with two fluorescent dyes showing optical compatibility, and were then analyzed with BD Pathway 855 (Becton Dickinson). By using the technology of HCS, cell parameters associated with nuclear morphology and caspase 3/7 activation (indicative of pre-lethal cytotoxic effects and representative of different mechanism of toxicity) were measured at the single cells level, which allows high-throughput screening. Data were expressed as means±SD of three separate wells and individual comparisons were made with Student's test (SigmaPlot 9.0 program).

According to the data obtained with the analysis of all studied parameters, compounds were classified in four groups according their degree of cardioprotection: No cardioprotection, low cardioprotection, moderate cardioprotection and high cardioprotection.

### Materials and Methods

Reagents and Equipment

| Reagent/Equipment and Catalogue | |
|---|---|
| and lot Numbers | Supplier |
| Luminescence ATP Detection Assay Kit (# | Abcam |
| Cat. FLAA-1KT) | |
| Myocyte Growth Supplement (# Cat. 6252) | Sciencell |
| FBS (# Cat. F7524) | Sigma Aldrich |
| Trypsin-EDTA 0.5% (w/v) (# Cat. 25300) | Gibco |
| DMSO (# Cat. D2650) | Sigma Aldrich |
| Penicillin/Streptomycin (# Cat. 15240) | Gibco |
| Centrifuge (# Allegra X-22R) | Beckman Coulter |
| Incubator (# Model 381 S/N 314342) | Thermo Scientific |
| PBS (# Cat. D8537) | Sigma Aldrich |
| Laminar flow cabinet (# ESCO class | Lab Culture |
| II BSC) | |
| Trypan blue 0.4% (# Cat. TI0282) | Invitrogen |
| Fibronectin (# Cat.) | Sciencell |
| Pathway 855 (no A107) | Becton Dickinson |
| LDH kit (# Cat 11644793001) | Roche |
| Cell event Caspase 3/7 green detection | Invitrogen |
| (# Cat. C10423) | |
| HOECHST (# Cat. H1399) | Invitrogen |
| Hypoxia chamber (# Cat. 5352414) | Billups-Rothenberg |
| Synergy II microplate reader | Biotek Instruments |
| Cell counting Kit-8 (# Cat. 96992) | Sigma-Aldrich |
| SILAC DMEM no glucose (# Cat. A24939-1) | Gibco |
| Neonatal heart dissociation kit (# Cat. | Miltenyi Biotec |
| 130-098-373) | |
| GentleMACS Dissociator (# Cat. | Miltenyi Biotec |
| 130-093-235) | |
| Neonatal cardiomyocytes isolation kit (# | Miltenyi Biotec |
| Cat. 130-100-825) | |

Test Compounds
“API”—Recrystallized oltipraz (Appearance: Red Powder (98.6% HPLC purity; weight 1 g)
“DPI”-Formulated oltipraz crystals prepared as described above in Example 1. The MHD of the crystals, as measured by dynamic light scattering (DLS), was about 300 nm. (Appearance: Red Powder; weight 1 g)
Atovaquone—Sigma Aldrich, weight 10 mg (solubilized in DMSO)

Isolation and Culture of Cardiomyocytes.

Primary cultures of cardiomyocytes were prepared from the hearts of Swiss mouse neonates P0-P2. Hearts were removed and enzymatically dissociated following instructions from Neonatal heart Dissociation Kit of Miltenyi Biotec. Then cardiomyocytes were isolated following also the instructions of Miltenyi biotec. Briefly, cells were isolated by depletion of non-target cells. Non-target cells (fibroblasts and endothelial cells) are directly magnetically labeled with a cocktail of monoclonal antibodies conjugated with MACS microbeads. The magnetically labeled non-target cells were depleted by retaining them within an MCS column in the magnetic fields of a MACS separator, while the unlabeled cardiomyocytes passed through the column. Then, cardiomyocytes were counted and plated on fibronectin-coated wells with cardiac myocyte complete medium (1% CMGS component, 1% penicillin/streplomycina and 5% FBS).

### Assay of Oxygen Glucose Deprivation Induced Toxicity.

Experimental Procedure.

Cardiomyocytes from P0-P2 days mice were plated in fibronectin coated 96-well plates with a number of 30.000 cells per well. Cells were maintained in cardiac myocyte complete medium (Cardiac myocyte medium, growth factors, 5% FBS, 1% penicillin/streptomycin) for 1 day at 37° C. in a humidified 5% CO2 atmosphere. At day 2, cells were washed and treated with compounds in complete medium 4 h prior to being subjected to oxygen glucose deprivation for 18 hours at 37° C. The cultures were placed in an aerobic chamber (Billops-Rothenberg) and incubated with DMEM medium lacking glucose and growth factors, and aerated with an anaerobic gas mix (94.7% N2, 5% CO2, 0.3% O2) to remove residual oxygen. Control cells were kept in the original cardiac myocyte medium but under anaerobic conditions. At the end of the OGD insult the cells were removed from the anaerobic chamber, the OGD medium was replaced with cardiac myocyte medium containing glucose without growth factors, and the cells were incubated for additional 24 h. The test compounds were also present during the OGD insult and the recovery period. Some cells were treated as described above with NAC 100 μM as positive control of cardioprotection.

HCS Assay: Incubation and Imaging of Fluorescent Probes.

Compound neuroprotective potential was determined by HCS analysis, which included the following endpoints: plasma membrane integrity, cell viability, alterations of mitochondrial membrane potential, caspase 3n activation and neurite outgrowth.

LDH Assay:

To determine the integrity of the plasma membrane, supernatants were collected 24 h after treatments and LDH assay was performed following manufacturer's instructions.

Cell Number:

Cell number was determined by hoechst 33342 nucleic acid staining. This dye allows a sensitive cell number determination by fluorescence microscopy. Cells were stained with 5 μg/ml, washed 3 times and measured at 380 nm/460 nm Ex/Em.

Caspase 3/7 Activation:

Caspase 3/7 activation was determined using The CellEvent® Caspase-3n Green Detection Reagent which is intrinsically a non-fluorescent peptide that inhibits the ability of the dye to bind to DNA. However, after caspase-37 activation in apoptotic cells, the peptide is cleaved allowing the dye to bind to DNA producing a bright, fluorogenic response. Cell were stained with 5 μM reagent, washed 3 times and measured at 488 nm/530 nm Ex/Em. This dye permits the direct quantification of apoptotic cells.

Cell Viability:

For detection of viable cells, 10 μl of CCK-8 reagent (WST-8) were added to each well and the plate was incubated at 37° C. After 1 hour, absorbance was measured at 450 nm using the Synergy II microplate reader.

Intracellular ATP

Intracellular ATP levels were determined using the Luminescence ATP Detection Assay Kit. After 24 h of reoxygenation the cells were lysed using a detergent provided with the kit that inactivates ATPases, ensuring the signal obtained truly corresponds to the levels of cellular ATP. The assay was performed following manufacturer's instructions.

At the end of the assay, for measuring viable cells, wells were first stained with WST-8 for 1 hour and intracellular ATP levels were determined, and then cells were simultaneously loaded with fluorescent dyes (cellEvent® Caspase-3/7 Green Detection Reagent for caspase measure and HOECHST for cell number measure). After 1 h of incubation at 37° C. with the culture media containing the fluorescent probes, cells were imaged using BD Pathway 855, and the attovision analyzer system was employed. In order to acquire enough cells for the analysis, nine fields per well were imaged. The 20× objective was used to collect images for the distinct fluorescence channels. Dyes were excited and their fluorescence was monitored at the excitation and emission wavelengths with appropriate filter settings. In the setting up of the procedure, exposure times were adjusted in order to avoid overlapping emission between different probes. The collected images were analyzed using a module that allows the simultaneous quantification of subcellular structures, which were stained by different molecular probes and measure the fluorescence intensity associated with predefined nuclear and cytoplasmic compartments.

Analysis of HCS Data.

For all the compounds and studied parameters, a variation of at least 20% in fluorescence intensity or in the corresponding morphological parameter in relation to vehicle-treated cultures under OGD-conditions was considered. In order to compare their degree of cardioprotection, the level of change for each parameter at 24 h was studied at each concentration. Four different scores of cardioprotection were established according to the level of variation when compared with control cells: 0 (no cardioprotection or variation lower than 20%), 1 (variation 20-40%), 2 (variation 40-60%) and 3 (variation 60-100%). The sum of each individual score resulted in the total level of cardioprotection for each compound and was defined as its degree of cardioprotection. From this calculation, a cardioprotective score was established: high (>5), moderate (3-4), low (1-2) and no cardioprotection (0).

### Results

Cardioprotection of Test Compounds Against OGD-Induced Toxicity

Cell Viability

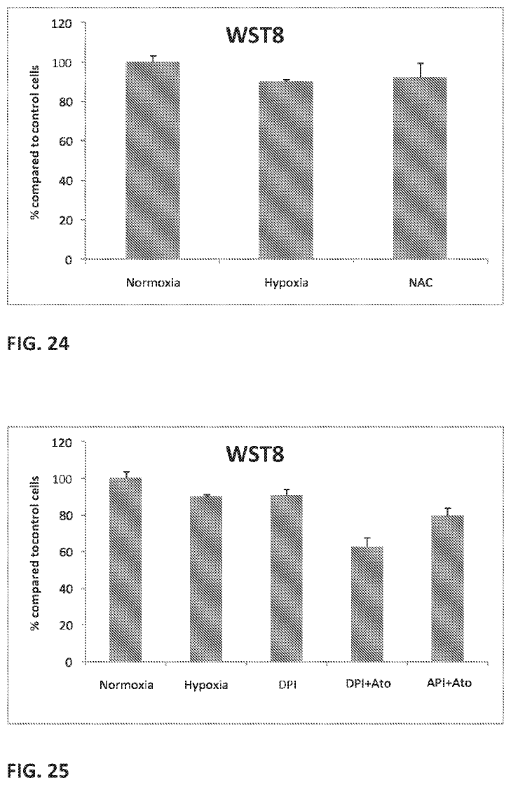

OGD treatment resulted in a decrease of 10% of cell viability measured by WST-8 in cardiomyocytes when measured 24 h after exposure to OGD conditions. Therefore, the effect size was too small to make conclusions on compound effects with the WST-8 assay (FIGS. 24 and 25).

Cell Number.

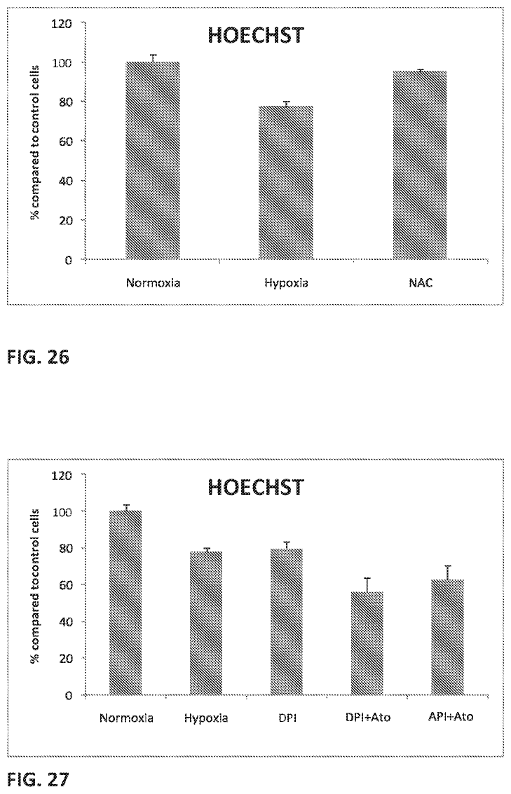

OGD treatment resulted in 23% of mortality in cardiomyocytes when measured 24 h after exposure. The positive control NAC prevented OGD-induced mortality by 21% (FIG. 26). OGD-induced cell death could be also prevented by application of test compound DPI. Therefore, compound supplementation significantly reduced cell death and improved survival of cardiomyocytes (FIG. 27).

It was observed that compounds DPI+Ato and API+Ato in addition to OGD induced a decrease in cell number.

Plasma Membrane Integrity.

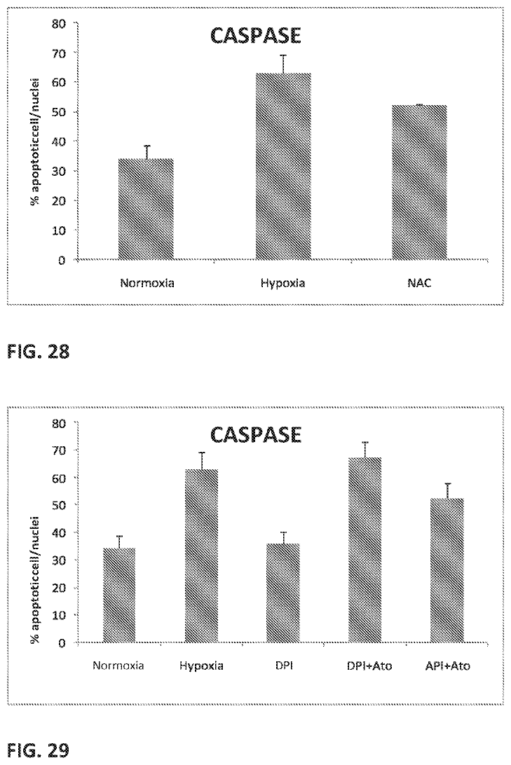

To study the effects of OGD on plasma membrane integrity, LDH quantification in supernatants of treated cells were employed. It was observed that OGD caused an increase of LDH of 59% in cultured cardiomyocytes, while the positive control (NAC) normalized OGD-induced LDH release by 26%. OGD-induced cell death was prevented by test compound DPI. Therefore, compound supplementation significantly reduced LDH release and improved survival of cardiomyocytes (FIGS. 28 and 29).

It was observed that compounds DPI+Ato and API+Ato in addition to OGD induced an increase in LDH production.

Apoptosis

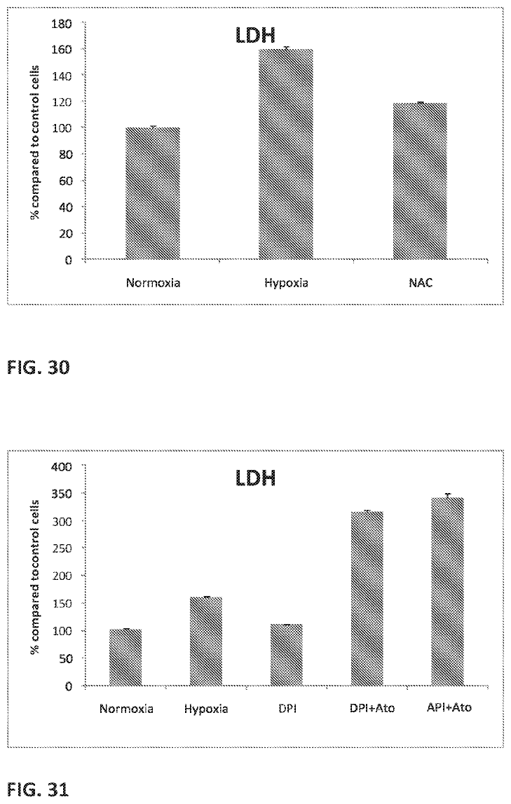

OGD treatment resulted in an increase of two-fold of caspase 3n activity in cardiomyocytes when measured 24 h after exposure. The positive control NAC prevented OGD-induced apoptosis by 31% (FIG. 30). Compounds DPI and API+Ato reduced OGD-induced caspase 3/7 activity and improved survival of cardiomyocytes (FIG. 31).

It was observed that compounds DPI+Ato and API+Ato in addition to OGD induced an increase in caspase 3n activation.

Intracellular ATP

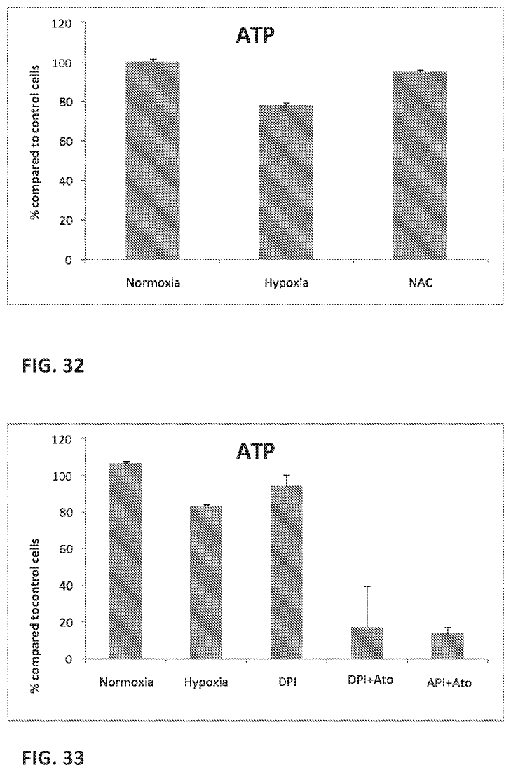

OGD treatment resulted in a 22% reduction in the ATP levels compared to control cells. The treatment with 100 μM of NAC protected the cells by 21%. Compound DPI could also prevented the OGD-induced ATP reduction (FIGS. 32 and 33).

It was observed that compounds DPI+Ato and API+Ato in addition to OGD induced a decrease in intracellular ATP.

In order to compare the degree of neuroprotection of tested compounds, different scores were created according to the level of the alteration (the group formation criterion is described in material and methods section) and then a value was assigned to each parameter. The sum of all the values for each parameter and concentration tested was calculated, and four distinct groups were created according to the level of neuroprotection provided. From this analysis, the degree of cardioprotection of each compound studied was defined (Table 17).

Briefly, DPI scored moderate cardioprotection degree at 100 μg/ml. DPI and API plus atovaquone scored no cardioprotection at 100 μg/ml and seemed to be toxic in combination with OGD. Positive control NAC exhibited moderate cardioprotection with the concentration tested (100 μM) (Table 18).

TABLE 17

| | DPI | DPI + ATO | API + ATO |
|---|---|---|---|
| HOECHST | 0 | 0 | 0 |
| CASPASE | 2 | 0 | 1 |
| ATP | 1 | 0 | 0 |
| LDH | 1 | −3 | −3 |
| Total | 4 | −3 | −2 |
| Neuroprotection degree | Moderate | Toxic | Toxic |

TABLE 18

| | NAC |
|---|---|
| HOECHST | 1 |
| CASPASE | 1 |
| ATP | 1 |
| LDH | 1 |
| Total | 4 |
| Neuroprotection degree | Moderate |

### Conclusion

In the present study OGD toxicity is linked to an increase in caspase 3/7 activation and LDH secretion. On the other hand, a decrease in cell number, viability and intracellular ATP is also present. The preventive effects shown by compounds against OGD toxicity are associated with increase of cell number, restoration of caspase 3/7 activity, stabilization of cell viability and intracellular ATP, and a decrease of LDH secretion.

Compound DPI scored moderate cardioprotection levels and based on the data appeared to be composition useful for preventing OGD-induced toxicity. The positive control NAC also scored moderate cardioprotection level.

As noted above in Example 18, the compounds separately dissolved correctly but when preparing the formulated oltipraz crystals plus atovaquone, and recrystallized oltipraz plus atovaquone, some red precipitates were observed. Precipitation was higher in the case of recrystallized oltipraz plus atovaquone solution and the precipitates could not be completely dissolved by heat and sonication. Accordingly, the actual level of cardioprotection provided by the formulated oltipraz crystals and recrystallized oltipraz through the addition of atovaquone, if completely dissolved through appropriate means, could be greater than that observed. Moreover, as also noted above, atovaquone is more readily absorbed with meals, as is oltipraz, and thus absorption is enhanced by lipids, e.g., vegetable oil, which may be reflected in the poor solubility as well. Additionally, the toxicity results in the above experiments for the compositions comprising atovaquone may simply reflect the limitations of the experimental model, as atovaquone is an approved drug that is safe for human use.

### Recitation of Embodiments

Embodiments 1-266 below are the embodiments described in PCT Application IB2017-001312 (“Formulations of 4-Methyl-5-(Pyrazin-2-yl)-3H-1,2-Dithiole-3-Thione, and Methods of Making and Using Same”; Applicant ST IP Holding AG), the disclosure of which is incorporated herein by reference. Embodiments 267-351 then describe the compositions and methods that comprise both 4-Methyl-5-(Pyrazin-2-yl)-3H-1,2-Dithiole-3-Thione and an OCR-API.
1. A composition comprising a quantity of crystals, wherein the quantity substantially comprises crystals of 4-methyl-5-(pyrazin-2-yl)-3H-1,2-dithiole-3-thione having an intensity averaged, mean hydrodynamic diameter (Z-average) (“MHD”) of from 30 to 2000 nm.
   wherein the MHD is determined performing dynamic light scattering at 25° C. on a suspension of the crystals in water at a concentration of 0.01 to 0.1 mg of crystals per mL of water.
2. A composition according to embodiment 1, wherein the quantity substantially comprises crystals that have a MHD in the range of from 30 to 100 nm.
3. A composition according to embodiment 1, wherein the quantity substantially comprises crystals that have a MHD in the range of from 100 to 1200 nm.
4. A composition according to embodiment 1, wherein the quantity substantially comprises crystals that have a MHD in the range of from 150 to 600 nm.
5. A composition according to embodiment 1, wherein the quantity substantially comprises crystals that have a MHD in the range of from 150 to 450 nm.
6. A composition according to embodiment 2, wherein the composition comprises at least one stabilizing agent, and wherein the quantity substantially comprises crystals that will have a MHD in the range of from 30 to 100 nm if left in water at 25° C. for 1 hour.
7. A composition according to embodiment 2, wherein the composition comprises at least one stabilizing agent, and wherein the quantity substantially comprises crystals that will have a MHD in the range of from 30 to 100 nm if left in water at 25° C. for 1 hour.
8. A composition according to embodiment 3, wherein the composition comprises at least one stabilizing agent, and wherein the quantity substantially comprises crystals that will have a MHD in the range of from 100 to 1200 nm if left in water at 25° C. for 1 hour.
9. A composition according to embodiment 3, wherein the composition comprises at least one stabilizing agent, and wherein the quantity substantially comprises crystals that will have a MHD in the range of from 100 to 1200 nm if left in water at 25° C. for 24 hours.
10. A composition according to embodiment 4, wherein the composition comprises at least one stabilizing agent, and wherein the quantity substantially comprises crystals that will have a MHD in the range of from 150 to 600 nm if left in water at 25° C. for 1 hour.
11. A composition according to embodiment 4, wherein the composition comprises at least one stabilizing agent, and wherein the quantity substantially comprises crystals that will have a MHD in the range of from 150 to 600 nm if left in water at 25° C. for 24 hours.
12. A composition according to embodiment 5, wherein the composition comprises at least one stabilizing agent, and wherein the quantity substantially comprises crystals that will have a MHD in the range of from 150 to 450 nm if left in water at 25° C. for 1 hour.
13. A composition according to any of embodiments 1-12, wherein the composition comprises less than 1 percent of drug-degradant impurities relative to 4-methyl-5-(pyrazin-2-yl)-3H-1,2-dithiole-3-thione in the aqueous composition and less than 2 percent total impurities relative to the 4-methyl-5-(pyrazin-2-yl)-3H-1,2-dithiole-3-thione in the aqueous suspension.
14. A composition according to any of embodiments 1-12, wherein the composition comprises less than 0.1 percent of drug-degradant impurities relative to 4-methyl-5-(pyrazin-2-yl)-3H-1,2-dithiole-3-thione in the aqueous composition and less than 0.5 percent total impurities relative to the 4-methyl-5-(pyrazin-2-yl)-3H-1,2-dithiole-3-thione in the aqueous suspension.
15. A composition according to any of embodiments 1-14, wherein the polydispersity index (PdI) of the crystals in the quantity is less than 0.80, wherein PdI=(a/d)², wherein σ is the standard deviation and d is the mean hydrodynamic diameter (Z-average).
16. A composition comprising a quantity of crystals according to embodiment 15, wherein the polydispersity index (PdI) of the crystals in the quantity is less than 0.60.
17. A composition comprising a quantity of crystals according to embodiment 15, wherein the polydispersity index (PdI) of the crystals in the quantity is between 0.10 and 0.60.
18. A composition comprising a quantity of crystals according to embodiment 15, wherein the polydispersity index (PdI) of the crystals in the quantity is between 0.10 and 0.45.
19. A composition according to any of embodiments 1-18, wherein the quantity of crystals comprises substantially the entire quantity of 4-methyl-5-(pyrazin-2-yl)-3H-1,2-dithiole-3-thione present in the composition.
20. A composition according to any of embodiments 5-19, wherein the at least one stabilizing agent comprises a polymer.
21. A composition according to embodiment 20, wherein the polymer is a cationic or anionic polymer.
22. A composition according to embodiment 21, wherein the polymer is a cationic polymer.
23. A composition according to embodiment 22, wherein the cationic polymer comprises ammonium functionality.
24. A composition according to embodiment 22, wherein the cationic polymer comprises quaternary ammonium functionality.
25. A composition according to any of embodiments 21-24, wherein the cationic polymer is a polymer that is formed from polymerization of compounds comprising at least one acrylate-containing compound.
26. A composition according to any of embodiments 21-25, wherein the cationic polymer comprises Poly(ethyl acrylate-co-methyl methacrylate-co-trimethylammonioethyl methacrylate chloride) 1:2:0.2 (Eudragit RL).
27. A composition according to any of embodiments 1-19, wherein the at least one stabilizing agent comprises a surfactant.
28. A composition according to any of embodiments 20-26, wherein the composition comprises a surfactant.
29. A composition according to embodiment 27 or 28, wherein the surfactant is a nonionic surfactant.
30. A composition according to embodiment 27-29, wherein the surfactant is a sorbitan ester.
31. A composition according to any of embodiments 27-30, wherein the surfactant is polyethylene glycol sorbitan monooleate.
32. A composition according to embodiment 27 or 28, wherein the surfactant is selected from the group consisting of polyethylene glycol sorbitan monooleate surfactants, polyethylene glycol hydrogenated castor oil, block copolymers of poly(ethylene oxide) and poly(propylene oxide), sodium lauryl sulfate, benzalkonium chloride, and sodium docusate.
33. A composition according to any of embodiments 1-19, wherein the composition comprises a bulking agent.
34. A composition according to any of embodiments 20-26, wherein the composition comprises a bulking agent.
35. A composition according to embodiment 27, wherein the composition comprises a bulking agent.
36. A composition according to any of embodiments 28-32, wherein the composition comprises a bulking agent.
37. A composition according to any of embodiments 5-19, wherein the at least one stabilizing agent comprises a bulking agent.
38. A composition according to any of embodiments 33-37, wherein the bulking agent comprises a polyvinylpyrrolidone compound.
39. A composition according to embodiment 38, wherein the bulking agent comprises a copolymer of polyvinylpyrrolidone and poly(vinyl acetate) with a ratio of approximately 6:4 of vinylpyrrolidone and vinyl acetate monomers (PVP-VA64).
40. A composition according to any of embodiments 1-19, wherein the composition comprises water.
41. A composition according to any of embodiments 1-19, wherein the composition comprises a non-aqueous solvent.
42. A composition according to embodiment 40 or 41, wherein the quantity of crystals comprise from 4 to 15 percent by weight of the composition.
43. A composition according to embodiment 40 or 41, wherein the quantity of crystals comprise from 6 to 12 percent by weight of the composition.
44. A composition according to embodiments 20-26, wherein the composition comprises water.
45. A composition according to any of embodiments 20-26, wherein the composition comprises a non-aqueous solvent.
46. A composition according to embodiment 44 or 45, wherein the quantity of crystals comprise from 4 to 15 percent by weight of the composition and the polymer comprises from 7.5 percent to 25 percent by weight of the composition.
47. A composition according to any of embodiments 27-32, wherein the composition comprises water.
48. A composition according to any of embodiments 27-32, wherein the composition comprises a non-aqueous solvent.
49. A composition according to embodiment 47 or 48, wherein the quantity of crystals comprises from 4 to 15 percent by weight of the composition and the surfactant comprises less than 5 percent by weight of the composition.
50. A composition according to any of embodiments 28-32, wherein the composition further comprises water.
51. A composition according to any of embodiments 28-32, wherein the composition comprises a non-aqueous solvent.
52. A composition according to embodiment 50 or 51, wherein the quantity of crystals comprises from 4 to 15 percent by weight of the composition, the polymer comprises from 2 to 10 percent by weight of the composition, and the surfactant comprises less than 5 percent by weight of the composition.
53. A composition according to any of embodiments 33-39, wherein the composition comprises water.
54. A composition according to any of embodiments 33-39, wherein the composition comprises a non-aqueous solvent.
55. A composition according to embodiment 53 or 54, wherein the quantity of crystals comprises from 1 to 10 percent by weight of the composition and the bulking agent comprises from 10 to 30 percent by weight of the composition.
56. A composition according to any of embodiments 34-39, wherein the composition comprises water.
57. A composition according to any of embodiments 34-39, wherein the composition comprises a non-aqueous solvent.
58. A composition according to embodiment 56 or 57, wherein the quantity of crystals comprises from 1 to 10 percent by weight of the composition, the polymer comprises less than 5 percent by weight of the composition, and the bulking agent comprises from 10 to 30 percent by weight of the composition.
59. A composition according to any of embodiments 35-39, wherein the composition comprises water.
60. A composition according to any of embodiments 35-39, wherein the composition comprises a non-aqueous solvent.
61. A composition according to embodiment 60 or 61, wherein the quantity of crystals comprises from 1 to 10 percent by weight of the composition, the surfactant comprises less than 4 percent by weight of the composition, and the bulking agent comprises from 10 to 30 percent by weight of the composition.
62. A composition according to any of embodiments 36-39, wherein the composition comprises water.
63. A composition according to any of embodiments 36-39, wherein the composition comprises a non-aqueous solvent.
64. A composition according to embodiment 61, wherein the quantity of crystals comprises from 1 to 10 percent by weight of the composition, the polymer comprises less than 5 percent by weight of the composition, the surfactant comprises less than 4 percent by weight of the composition, and the bulking agent comprises from 10 to 30 percent by weight of the composition.
65. A composition according to any of embodiments 33-39, wherein the composition substantially excludes water and any non-aqueous solvent.
66. A composition according to embodiment 65, wherein the bulking agent comprises from 50 to 90 percent by weight of the composition.
67. A composition according to embodiment 65, wherein the bulking agent comprises from 60 to 85 percent by weight of the composition.
68. A composition according to any of embodiments 34-39, wherein the composition substantially excludes water and any non-aqueous solvent.
69. A composition according to embodiment 68, wherein the bulking agent comprises from 50 to 90 percent by weight of the composition, and the polymer comprises from 3 to 12 percent by weight of the composition.
70. A composition according to any of embodiments 35-39, wherein the composition substantially excludes water and any non-aqueous solvent.
71. A composition according to embodiment 70, wherein the bulking agent comprises from 50 to 90 percent by weight of the composition, and the surfactant comprises from 1 to 8 percent by weight of the composition.
72. A composition according to embodiment 70, wherein the bulking agent comprises from 60 to 85 percent by weight of the composition, and the surfactant comprises from 1 to 6 percent by weight of the composition.
73. A composition according to any of embodiments 36-39, wherein the composition substantially excludes water and any non-aqueous solvent.
74. A composition according to embodiment 73, wherein the bulking agent comprises from 50 to 90 percent by weight of the composition, the polymer comprises from 3 to 12 percent by weight of the composition, and the surfactant comprises from 1 to 8 percent by weight of the composition.
75. A composition according to embodiment 73, wherein the bulking agent comprises from 60 to 85 percent by weight of the composition, the polymer comprises from 5 to 10 percent by weight of the composition, and the surfactant comprises from 1 to 6 percent by weight of the composition.
76. A composition according to any of embodiments 64-75, wherein the quantity of crystals comprises from 5 to 25 percent by weight of the composition.
77. A composition according to any of embodiments 64-75, wherein the quantity of crystals comprises from 10 to 20 percent by weight of the composition.
78. A composition according to any of embodiments 64-75, wherein the quantity of crystals comprises from 12 to 17 percent by weight of the composition.
79. A composition according to any of embodiments 64-75, wherein the composition substantially excludes water, and wherein the composition is capable of forming a substantially complete aqueous suspension of a quantity of crystals.
80. A composition according to embodiment 79, wherein the composition will form a substantially complete aqueous suspension with vigorous shaking in less than 15 minutes if mixed with water at a weight: weight ratio of 1 part of the dry composition per 10 parts of water at 25° C.
81. A composition according to embodiment 79, wherein the composition will form a substantially complete aqueous suspension with vigorous shaking in less than 10 minutes if mixed with water at a weight: weight ratio of 1 part of the dry composition per 10 parts of water at 25° C.
82. A composition according to embodiment 79, wherein the composition will form a substantially complete aqueous suspension with vigorous shaking in less than 5 minutes if mixed with water at a weight: weight ratio of 1 part of the dry composition per 10 parts of water at 25° C.
83. A composition according to embodiment 79, wherein the composition will form a substantially complete aqueous suspension with vigorous shaking in less than 2 minutes if mixed with water at a weight: weight ratio of 1 part of the dry composition per 10 parts of water at 25° C.
84. A composition according to any of embodiment 79, wherein the composition will form a substantially complete aqueous suspension with vigorous shaking in less than 1 minute if mixed with water at a weight: weight ratio of 1 part of the dry composition per 10 parts of water at 25° C.
85. A composition according to any of embodiments 64-84 that substantially excludes water, wherein the composition has been made by a process comprising spray drying.
86. A composition that substantially excludes water made by a process comprising spray drying an aqueous formulation comprising water and a composition according to any of embodiments 1-63.
87. A composition according to any of embodiments 64-84 that substantially excludes water, wherein the composition has been made by a process comprising lyophilization.
88. A composition that substantially excludes water made by a process comprising lyophilizing an aqueous formulation comprising water and a composition according to any of embodiments 1-63.
89. A dry pharmaceutical composition comprising a composition according to any of embodiments 1-39 and 64-88 that substantially excludes water and any non-aqueous solvent.
90. A dry pharmaceutical composition according to embodiment 89, comprising at least one pharmaceutically acceptable additive.
91. A dry pharmaceutical composition according to embodiment 89 or 90, comprising a pharmaceutically acceptable additive that inhibits microbial growth.
92. A dry pharmaceutical composition according to any of embodiments 89-91, comprising a pharmaceutically acceptable lubricant.
93. A dry pharmaceutical composition according to embodiment 92, wherein the lubricant is magnesium stearate or silica oxide.
94. A dry pharmaceutical composition according to embodiment 92 or 93, wherein the lubricant is present in an amount of up to 2 percent by weight of the pharmaceutical composition.
95. A dry pharmaceutical composition comprising up to 2000 mg of a dry pharmaceutical composition according to any of embodiments 89-94.
96. A dry pharmaceutical composition comprising up to 1000 mg of a dry pharmaceutical composition according to any of embodiments 89-94.
97. A dry pharmaceutical composition comprising up to 500 mg of a dry pharmaceutical composition according to any of embodiments 89-94.
98. A pharmaceutical composition suitable for oral administration comprising a liquid and a composition according to any of embodiments 1-63.
99. A pharmaceutical composition suitable for oral administration comprising a non-aqueous liquid and a composition according to any of embodiments 1-63.
100. An aqueous pharmaceutical composition suitable for oral administration comprising water and a composition according to any of embodiments 1-63.
101. An liquid pharmaceutical composition prepared by a process comprising the step of mixing a combination of ingredients comprising a liquid and a dry pharmaceutical composition according to any of embodiments 89-97.
102. A pharmaceutical composition according to 101, wherein the mixture comprises, in a weight: weight ratio, 1 part of dry pharmaceutical composition and up to 100 parts of liquid.
103. A pharmaceutical composition according to 101, wherein the mixture comprises, in a weight: weight ratio, 1 part of dry pharmaceutical composition and up to 60 parts of liquid.
104. A pharmaceutical composition according to 101, wherein the mixture comprises, in a weight: weight ratio, 1 part of dry pharmaceutical composition and up to 40 parts of liquid.
105. A pharmaceutical composition according to 101, wherein the mixture comprises, in a weight: weight ratio, 1 part of dry pharmaceutical composition and up to 20 parts of water.
106. A pharmaceutical composition according to any of embodiments 101-105, comprising at least one pharmaceutically acceptable taste-modifying additive.
107. A pharmaceutical composition according to any of embodiments 101-106, wherein the liquid comprises water.
108. A pharmaceutical composition according to any of embodiments 101-107, wherein the liquid comprises a non-aqueous solvent.
109. A pharmaceutical composition for topical administration to skin comprising a composition according to any of embodiments 1-78 and a pharmaceutically acceptable ingredient for topical administration.
110. A pharmaceutical composition for rectal administration comprising a composition according to any of embodiments 1-78 and a pharmaceutically acceptable ingredient for rectal administration.
111. A pharmaceutical composition for colonic administration comprising a composition according to any of embodiments 1-78 and a pharmaceutically acceptable ingredient for colonic administration.
112. A pharmaceutical composition for administration by inhalation comprising a composition according to any of embodiments 1-39 and 64-99.
113. A medical device comprising an inhaler and a pharmaceutical composition for administration by inhalation according to embodiment 112.
114. A process for making a medical device according to embodiment 113 comprising loading a dose of a pharmaceutical composition according to embodiment 112 into an inhaler.
115. A pharmaceutical dose for oral administration comprising a composition according to any of embodiments 1-39 and 64-99.
116. A pharmaceutical composition according to embodiment 115, wherein the dose is in the form of a pills, tablet or capsule that substantially excludes water.
117. A pharmaceutical composition according to embodiment 115, wherein the dose is in the form of a liquid.
118. A pharmaceutical composition according to embodiment 117, wherein the dose is in a soft gel capsule.
119. A pharmaceutically acceptable container for providing an aqueous pharmaceutical composition, comprising a cavity of sufficient size to hold both a dry pharmaceutical composition and a quantity of liquid sufficient to permit mixing of the dry pharmaceutical composition to form a liquid composition, wherein the dry pharmaceutical composition comprises a composition according to any of embodiments 1-39 and 64-97 that substantially excludes water and any non-aqueous solvents.
120. A pharmaceutically acceptable container according to embodiment 119, further comprising a releasable coupling for providing an opening in the container adapted to dispense a liquid composition from the container.
121. A pharmaceutically acceptable container according to embodiment 119, comprising a compartment separate from the cavity, said compartment comprising the dose of the dry pharmaceutical composition.
122. A pharmaceutically acceptable container according to embodiment 120, comprising a compartment separate from the cavity, said compartment comprising the dose of the dry pharmaceutical composition.
123. A pharmaceutically acceptable container according to embodiment 122, wherein the releasable coupling connects the portion of the container comprising the cavity to the portion of the container comprising the compartment.
124. A pharmaceutically acceptable container according to any of embodiments 120-123, further comprising a breakable seal between the compartment and the cavity.
125. A pharmaceutically acceptable container according to any of embodiments 119-124, further comprising a liquid comprising water.
126. A pharmaceutically acceptable container according to any of embodiments 119-125, further comprising a liquid comprising a non-aqueous solvent.
127. A pharmaceutically acceptable container according to embodiment 125 or 126, wherein the liquid also comprises at least one pharmaceutically acceptable taste-modifying additive.
128. A process comprising the step of wet milling a composition comprising a liquid and 4-methyl-5-(pyrazin-2-yl)-3H-1,2-dithiole-3-thione to form a liquid composition comprising crystals of 4-methyl-5-(pyrazin-2-yl)-3H-1,2-dithiole-3-thione.
   wherein wet milling yields a quantity of crystals that have an intensity averaged, mean hydrodynamic diameter (Z-average) (“MHD”) of from 100 to 2000, wherein the MHD is determined by dynamic light scattering at 25° C. and a concentration of 0.01 to 0.1 mg of crystals per mL of water.
129. A process according to embodiment 128, wherein wet milling yields a quantity of crystals that have a MHD of from 100 to 1200 nm.
130. A process according to embodiment 128, wherein wet milling yields a quantity of crystals that have a MHD of from 150 to 600 nm.
131. A process according to embodiment 128, wherein wet milling yields a quantity of crystals that have a MHD of from 150 to 450 nm.
132. A process according to embodiments 128, wherein the composition comprises at least one stabilizing agent, and wherein the quantity of crystals will have a MHD of from 100 to 2000 nm if left in water at 25° C. for at least 1 hour.
133. A process according to embodiments 132, wherein the quantity of crystals will have a MHD in the range of from 100 to 2000 nm if left in water at 25° C. for 24 hours.
134. A process according to embodiments 129, wherein the composition comprises at least one stabilizing agent, and wherein the quantity of crystals will have a MHD of from 100 to 1200 nm if left in water at 25° C. for at least 1 hour.
135. A process according to embodiments 134, wherein the quantity of crystals will have a MHD of from 100 to 1200 nm if left in water at 25° C. for 24 hours.
136. A process according to embodiments 130, wherein the composition comprises at least one stabilizing agent, and wherein the quantity of crystals will have a MHD of from 150 to 600 nm if left in water at 25° C. for at least 1 hour.
137. A process according to embodiments 136, wherein the quantity of crystals will have a MHD of from 150 to 600 nm if left in water at 25° C. for 24 hours.
138. A process according to embodiments 131, wherein the composition comprises at least one stabilizing agent, and wherein the quantity of crystals will have a MHD of from 150 to 450 nm if left in water at 25° C. for at least 1 hour.
139. A process according to embodiments 138, wherein the quantity of crystals will have a MHD of from 150 to 4500 nm if left in water at 25° C. for 24 hours.
140. A process according to any of embodiments 132-140, wherein the stabilizing agent comprises a polymer.
141. A process according to embodiment 140, wherein the polymer comprises a cationic or anionic polymer.
142. A process according to embodiment 140, wherein the polymer is a cationic polymer.
143. A process according to embodiment 142, wherein the cationic polymer comprises ammonium functionality.
144. A process according to embodiment 143, wherein the cationic polymer comprises quaternary ammonium functionality.
145. A process according to any of embodiments 141-144, wherein the cationic polymer comprises a polymer that is formed from polymerization of compounds comprising at least one acrylate-containing compound.
146. A process according to any of embodiments 141-145, wherein the cationic polymer comprises Poly(ethyl acrylate-co-methyl methacrylate-co-trimethylammonioethyl methacrylate chloride) 1:2:0.2 (Eudragit RL).
147. A process according to any of embodiments 132-146, wherein the stabilizing agent comprises between 10 and 20 percent by weight of the liquid composition.
148. A process according to any of embodiments 132-146, wherein the stabilizing agent comprises between 12 and 17 percent by weight of the liquid composition.
149. A process according to any of embodiments 128-148, wherein the liquid composition comprises a surfactant.
150. A process according to embodiment 149, wherein the surfactant is selected from the group consisting of polyethylene glycol sorbitan monooleate surfactants, polyethylene glycol hydrogenated castor oil, block copolymers of poly(ethylene oxide) and poly(propylene oxide), sodium lauryl sulfate, benzalkonium chloride, and sodium docusate.
151. A process according to embodiment 149 or 150, wherein the surfactant comprises a nonionic surfactant.
152. A process according to any of embodiments 149-151, wherein the surfactant comprises a sorbitan ester.
153. A process according to any of embodiments 149-152, wherein the surfactant is polyethylene glycol sorbitan monooleate.
154. A process according to any of embodiments 149-153, wherein the surfactant comprises from 1 to 5 percent by weight of the composition.
155. A process according to any of embodiments 149-153, wherein the surfactant comprises from 1 to 3 percent by weight of the composition.
156. A process according to any of embodiments 128-155, wherein the quantity of crystals of 4-methyl-5-(pyrazin-2-yl)-3H-1,2-dithiole-3-thione comprise less than 20 percent by weight of the liquid composition.
157. A process according to any of embodiments 128-155, wherein the quantity of crystals of 4-methyl-5-(pyrazin-2-yl)-3H-1,2-dithiole-3-thione comprise less than 15 percent by weight of the liquid composition.
158. A process according to any of embodiments 128-155, wherein the quantity of crystals of 4-methyl-5-(pyrazin-2-yl)-3H-1,2-dithiole-3-thione comprise from 5 to 12 percent by weight of the liquid composition.
159. A process according to any of embodiments 128-155, wherein the quantity of crystals of 4-methyl-5-(pyrazin-2-yl)-3H-1,2-dithiole-3-thione comprise from 7 to 10 percent by weight of the liquid composition.
160. A process according to any of embodiments 128-159, further comprising the step of combining a bulking agent with at least a portion of said liquid composition to form a liquid composition comprising the bulking agent and crystals.
161. A process according to embodiment 160, wherein the step of combining a bulking agent comprises mixing the bulking agent and a liquid composition comprising the crystals to form a liquid composition comprising the bulking agent and crystals.
162. A process according to any of embodiments 160-161, wherein the bulking agent comprises a polyvinylpyrrolidone compound.
163. A process according to any of embodiments 160-162, wherein the bulking agent comprises a copolymer of polyvinylpyrrolidone and poly(vinyl acetate) with a ratio of approximately 6:4 of vinylpyrrolidone and vinyl acetate monomers (PVP-VA64).
164. A process according to any of embodiments 128-163, wherein the liquid composition comprises water.
165. A process according to any of embodiments 128-164, wherein the liquid composition comprises a non-aqueous solvent.
166. A process according to any of embodiments 160-165, comprising the step of adding a liquid comprising water to adjust the percent solids content of the liquid composition comprising the bulking agent and crystals.
167. A process according to any of embodiments 160-166, wherein the bulking agent comprises less than 30 percent by weight of the liquid composition comprising the bulking agent and crystals.
168. A process according to any of embodiments 160-166, wherein the bulking agent comprises between 15 and 25 percent by weight of the liquid composition comprising the bulking agent and crystals.
169. A process according to any of embodiments 160-168, wherein the liquid composition comprising the bulking agent and crystals comprises more than 35 percent total solids.
170. A process according to any of embodiments 160-168, wherein the liquid composition comprising the bulking agent and crystals comprises from 30 to 35 percent total solids.
171. A process according to any of embodiments 160-168, wherein the liquid composition comprising the bulking agent and crystals comprises from 25 to 30 percent total solids.
172. A process according to any of embodiments 160-168, wherein the liquid composition comprising the bulking agent and crystals comprises from 20 to 25 percent total solids.
173. A process according to any of embodiments 160-168, wherein the liquid composition comprising the bulking agent and crystals comprises from 15 to 20 percent total solids.
174. A process according to any of embodiments 160-168, wherein the liquid composition comprising the bulking agent and crystals comprises less than 15 percent total solids.
175. A process according to any of embodiments 160-168, wherein the liquid composition comprising the bulking agent and crystals comprises about 28 percent total solids.
176. A process according to any of embodiments 160-175 further comprising one or more steps to form a dry composition that substantially excludes liquid, wherein the one or more steps comprise the step of spray drying the liquid composition comprising the bulking agent and crystals.
177. A process according to any of embodiments 160-175 further comprising one or more steps to form a dry composition that substantially excludes liquid, wherein the one or more steps comprise the step of lyophilizing the liquid composition comprising the bulking agent and crystals.
178. A process according to any of embodiments 128-175, wherein the liquid composition comprises less than 1 percent of drug-degradant impurities relative to 4-methyl-5-(pyrazin-2-yl)-3H-1,2-dithiole-3-thione in the liquid composition and less than 2 percent total impurities relative to the 4-methyl-5-(pyrazin-2-yl)-3H-1,2-dithiole-3-thione in the liquid composition.
179. A process according to any of embodiments 128-175, wherein the liquid composition comprising the bulking agent and crystals comprises less than 0.5 percent of drug-degradant impurities relative to 4-methyl-5-(pyrazin-2-yl)-3H-1,2-dithiole-3-thione in the liquid composition and less than 1 percent total impurities relative to the 4-methyl-5-(pyrazin-2-yl)-3H-1,2-dithiole-3-thione in the liquid composition.
180. A process according to any of embodiments 176 or 177, wherein the dry composition comprises less than 1 percent of drug-degradant impurities relative to 4-methyl-5-(pyrazin-2-yl)-3H-1,2-dithiole-3-thione in the liquid composition and less than 2 percent total impurities relative to the 4-methyl-5-(pyrazin-2-yl)-3H-1,2-dithiole-3-thione in the liquid composition.
181. A process according to any of embodiments 176 or 177, wherein the dry composition comprising the bulking agent and crystals comprises less than 0.5 percent of drug-degradant impurities relative to 4-methyl-5-(pyrazin-2-yl)-3H-1,2-dithiole-3-thione in the liquid composition and less than 1 percent total impurities relative to the 4-methyl-5-(pyrazin-2-yl)-3H-1,2-dithiole-3-thione in the liquid composition.
182. A process according to any of embodiments 128-181, wherein the polydispersity index (PdI) of the crystals in the quantity is less than 0.80, wherein PdI=(a/d)², wherein σ is the standard deviation and d is the mean hydrodynamic diameter (Z-average).
183. A process according to embodiment 182 wherein the polydispersity index (PdI) of the crystals in the quantity is less than 0.60.
184. A process according to embodiment 182, wherein the polydispersity index (PdI) of the crystals in the quantity is between 0.10 and 0.60.
185. A process according to embodiment 182, wherein the polydispersity index (PdI) of the crystals in the quantity is between 0.10 and 0.45.
186. A process according to any of embodiments 128-185, wherein the quantity of crystals comprises substantially the entire quantity of 4-methyl-5-(pyrazin-2-yl)-3H-1,2-dithiole-3-thione present in the liquid composition.
187. A process comprising the steps of
   providing a pharmaceutically acceptable container comprising a cavity, and
   adding to the container a dose of a dry pharmaceutical composition, wherein the dry pharmaceutical composition comprises a composition according to any of embodiments 1-39 and 64-99 that substantially excludes water,
   wherein the cavity is of sufficient size to hold both the dry pharmaceutical composition and an amount of a liquid sufficient to permit mixing of the dry pharmaceutical composition with a liquid to form a liquid pharmaceutical composition.
188. A process comprising the steps of
   providing a pharmaceutically acceptable container comprising a cavity, and
   adding to the container a dose of a dry pharmaceutical composition, wherein the dry pharmaceutical composition comprises a dry composition prepared according to embodiment 176 or 177.
   wherein the cavity is of sufficient size to hold both the dry pharmaceutical composition and an amount of liquid sufficient to permit mixing of the dry pharmaceutical composition with a liquid to form a liquid pharmaceutical composition.
189. A process according to embodiment 187 or 188, wherein the container comprises a compartment separate from the cavity, and the dry pharmaceutical composition is added to the compartment.
190. A process according to any of embodiments 187-189, wherein the container comprises a releasable coupling for uncoupling a portion of the container to provide an opening for dispensing a liquid pharmaceutical composition from the container.
191. A process according to embodiment 189, wherein the container further comprises a releasable coupling for uncoupling a portion of the container to provide an opening for dispensing a liquid composition from the container, and wherein the releasable coupling connects the portion of the container comprising the cavity to the portion of the container comprising the compartment that contains the dose of a dry pharmaceutical composition.
192. A process according to any of embodiments 189-191, wherein the container further comprises a breakable seal between the compartment and the cavity, and wherein the dry pharmaceutical composition remains separate from the cavity when said seal is unbroken, and wherein the dry pharmaceutical composition can enter the cavity when the seal is broken.
193. A process according to any of embodiments 187-192, further comprises adding a liquid to the pharmaceutically acceptable container and mixing the liquid and dry pharmaceutical composition.
194. A process comprising the steps of adding a liquid to the cavity of a pharmaceutically acceptable container according to any of embodiments 119-124, and mixing the dose of dry pharmaceutical composition with the liquid.
195. A process comprising the steps of adding a liquid to the cavity of a pharmaceutically acceptable container according to embodiment 122-124, causing the dry pharmaceutical composition in the compartment to enter the cavity, and mixing the dose of dry pharmaceutical composition with the liquid.
196. A process comprising the steps of adding a liquid to the cavity of a pharmaceutically acceptable container according to embodiment 124, breaking the seal between the compartment and the cavity and causing the dry pharmaceutical composition in the compartment to enter the cavity, and mixing the dose of dry pharmaceutical composition with the liquid.
197. A process according to any of embodiments 193-196, wherein the liquid further comprises at least one pharmaceutically acceptable taste-modifying additive.
198. A process according to any of embodiments 193-197, wherein the step of mixing is carried out by shaking the container for ten minutes or less.
199. A process according to any of embodiments 193-197, wherein the step of mixing is carried out by shaking the container for five minutes or less.
200. A process according to any of embodiments 193-197, wherein the step of mixing is carried out by shaking the container for three minutes or less.
201. A process according to any of embodiments 193-197, wherein the step of mixing is carried out by shaking the container for two minutes or less.
202. A process according to any of embodiments 193-197, wherein the step of mixing is carried out by shaking the container for one minute or less.
203. A process according to any of embodiments 193-202, wherein the liquid comprises water.
204. A process according to any of embodiments 193-203, wherein the liquid comprises a non-aqueous solvent.
205. A process for treating a human or non-human animal patient in need comprising administering to the patient a composition prepared according to the process of any of embodiments 193-204.
206. A process for treating a human or non-human animal patient in need comprising administering to the patient a pharmaceutical composition according to any of embodiments 98-108.
207. A process according to embodiment 205 or 206, wherein the administration comprises an oral administration.
208. A process according to embodiment 207, wherein the administration comprises a buccal administration
209. A process according to embodiment 208, wherein the buccal administration comprises a swish and swallow administration.
210. A process according to embodiment 208, where the buccal administration comprises a swish and spit administration.
211. A process for treating a human or non-human animal patient in need comprising administering to the patient a pharmaceutical composition according to any of embodiments 109-112.
212. A process for preventing, treating, ameliorating, lessening the severity and/or shortening the duration of mucositis for a human or non-human animal patient in need comprising orally administering a pharmaceutical composition according to any of embodiments 115-118 to the patient.
213. A process for preventing, treating, ameliorating, lessening the severity and/or shortening the duration of mucositis for a human or non-human animal patient in need comprising orally administering an pharmaceutical composition prepared according to any of embodiments 193-204.
214. A process for preventing, treating, ameliorating, lessening the severity and/or shortening the duration of mucositis for a human or non-human animal patient in need comprising orally administering to the patient a pharmaceutical composition according to any of embodiments 98-108.
215. A process according to any of embodiments 212-214, wherein the mucositis in oral mucositis.
216. A process according to any of embodiments 212-214, wherein the mucositis in mucositis of the alimentary canal.
217. A process for preventing, treating, ameliorating, lessening the severity and/or shortening the duration of mucositis for a human or non-human animal patient in need comprising topically administering a composition according to embodiment 109.
218. A process for preventing, treating, ameliorating, lessening the severity and/or shortening the duration of mucositis for a human or non-human animal patient in need comprising rectally administering a composition according to embodiment 110 or 111.
219. A process for preventing, treating, ameliorating, lessening the severity and/or shortening the duration of mucositis for a human or non-human animal patient in need comprising administering by inhalation a composition according to embodiment 112.
220. A process according to any of embodiments 205-219, wherein the patient is undergoing radiation therapy.
221. A process according to embodiment 222, wherein the patient receives administration prior to the patient receiving his or her next radiation treatment.
222. A process according to embodiment 221, wherein administration is carried out one hour or less prior to the patient receiving a radiation treatment.
223. A process according to embodiment 221, wherein administration is carried out one day or less prior to the patient receiving a radiation treatment.
224. A process according to any of embodiments 220-223, wherein administration is carried out after the patient receives a radiation treatment.
225. A process according to embodiment 224, wherein administration is carried out within one hour after the patient receives a radiation treatment.
226. A process according to embodiment 224, wherein administration is carried out less within one day after the patient receives a radiation treatment.
227. A process according to any of embodiments 205-226, wherein the composition comprising oltipraz is co-administered with at least one pharmaceutically acceptable agent selected from the group consisting of antioxidants, agents that enhance glutathione synthesis, glutathione. Medihoney. NF-kappaB inhibitors, anti-inflammatory agents, and compounds prevent damage from reactive O₂⁻ (superoxide).
228. A process according to any of embodiments 205-226, wherein the composition comprising oltipraz is co-administered with at least one pharmaceutically acceptable agent selected from the group consisting of N acetylcysteine, pantothenic acid (vitamin B5), glutathione. Medihoney, curcumin, Mesalamine, and superoxide dismutase.
229. A process according to any of embodiments 205-226, wherein the composition comprising oltipraz is co-administered separately as part of a dosing regimen with at least one pharmaceutically acceptable agent selected from the group consisting of antioxidants, agents that enhance glutathione synthesis, glutathione, Medihoney. NF-kappaB inhibitors, anti-inflammatory agents, and compounds prevent damage from reactive 0-(superoxide).
230. A process according to any of embodiments 205-226, wherein the composition comprising oltipraz is co-administered separately as part of a dosing regimen with at least one pharmaceutically acceptable agent selected from the group consisting of N acetylcysteine, pantothenic acid (vitamin B5), glutathione, Medihoney, curcumin, Mesalamine, and superoxide dismutase.
231. A composition according to any of embodiments 40-112, wherein the solubility of the crystals of 4-methyl-5-(pyrazin-2-yl)-3H-1,2-dithiole-3-thione in the composition is at least 5.0 μg/ml of water at 20° C.
232. A composition according to any of embodiments 40-108, wherein the solubility of the crystals of 4-methyl-5-(pyrazin-2-yl)-3H-1,2-dithiole-3-thione in the composition is at least 5.5 μg/ml of water at 20° C.
233. A composition according to any of embodiments 40-108, wherein the solubility of the crystals of 4-methyl-5-(pyrazin-2-yl)-3H-1,2-dithiole-3-thione in the composition is between 5.5 μg/ml of water and 6.0 μg/ml of water at 20° C.
234. A composition according to any of embodiments 40-108, wherein the solubility of the crystals of 4-methyl-5-(pyrazin-2-yl)-3H-1,2-dithiole-3-thione in the composition is between 6.0 μg/ml of water and 8.0 μg/ml of water at 20° C.
235. A pharmaceutically acceptable container according to any of embodiments 119-127, wherein the solubility of the crystals of 4-methyl-5-(pyrazin-2-yl)-3H-1,2-dithiole-3-thione in the composition is at least 5.0 μg/ml of water at 20° C.
236. A pharmaceutically acceptable container according to any of embodiments 119-127, wherein the solubility of the crystals of 4-methyl-5-(pyrazin-2-yl)-3H-1,2-dithiole-3-thione in the composition is at least 5.5 μg/ml of water at 20° C.
237. A pharmaceutically acceptable container according to any of embodiments 119-127, wherein the solubility of the crystals of 4-methyl-5-(pyrazin-2-yl)-3H-1,2-dithiole-3-thione in the composition is between 5.5 μg/ml of water and 6.0 μg/ml of water at 20° C.
238. A pharmaceutically acceptable container according to any of embodiments 119-127, wherein the solubility of the crystals of 4-methyl-5-(pyrazin-2-yl)-3H-1,2-dithiole-3-thione in the composition is between 6.0 μg/ml of water and 8.0 μg/ml of water at 20° C.
239. A process according to any of embodiments 128-230, wherein the solubility of the crystals of 4-methyl-5-(pyrazin-2-yl)-3H-1,2-dithiole-3-thione is at least 5.0 μg/ml of water at 20° C.
240. A process according to any of embodiments 128-230, wherein the solubility of the crystals of 4-methyl-5-(pyrazin-2-yl)-3H-1,2-dithiole-3-thione is at least 5.5 μg/ml of water at 20° C.
241. A process according to any of embodiments 128-230, wherein the solubility of the crystals of 4-methyl-5-(pyrazin-2-yl)-3H-1,2-dithiole-3-thione is between 5.5 μg/ml of water and 6.0 μg/ml of water at 20° C.
242. A process according to any of embodiments 128-230, wherein the solubility of the crystals of 4-methyl-5-(pyrazin-2-yl)-3H-1,2-dithiole-3-thione is between 6.0 and 8.0 μg/ml of water at 20° C.
243. A process for increasing the gene expression of glutathione peroxidase 4 (GPX4) and/or myeloperoxidase (MPO) in a human or non-human animal patient comprising administering a pharmaceutical composition according to any of the embodiments 89-112 to the patient.
244. A process for decreasing the gene expression of Peroxiredoxin 2 (PRDX2) in a human or non-human animal patient comprising administering a pharmaceutical composition according to any of the embodiments 89-112 to the patient.
245. A process for increasing the gene expression of glutathione peroxidase 4 (GPX4) and/or myeloperoxidase (MPO) and decreasing the gene expression of Peroxiredoxin 2 (PRDX2) in a human or non-human animal patient comprising administering a pharmaceutical composition according to any of the embodiments 89-112 to the patient.
246. A process for increasing the gene expression of glutathione peroxidase 4 (GPX4) and/or myeloperoxidase (MPO) in a human or non-human animal patient comprising administering a pharmaceutical composition according to any of the embodiments 187-204 to the patient.
247. A process for decreasing the gene expression of Peroxiredoxin 2 (PRDX2) in a human or non-human animal patient comprising administering a pharmaceutical composition according to any of the embodiments 187-204 to the patient.
248. A process for increasing the gene expression of glutathione peroxidase 4 (GPX4) and/or myeloperoxidase (MPO) and decreasing the gene expression of Peroxiredoxin 2 (PRDX2) in a human or non-human animal patient comprising administering a pharmaceutical composition according to any of the embodiments 187-204 to the patient.
249. A process according to any of embodiments 243 to 248, wherein the patient is undergoing chemotherapy and/or radiation therapy.
250. A process according to embodiment 249, wherein the patient has mucositis.
251. A process according to embodiment 250, wherein the mucositis is oral mucositis.
252. A process for decreasing intracellular reactive oxygen species (ROS) and/or decreasing oxidative stress in a human or non-human animal patient comprising administering a pharmaceutical composition according to any of the embodiments 89-112 to the patient.
253. A process for decreasing intracellular reactive oxygen species (ROS) and/or decreasing oxidative stress in a human or non-human animal patient comprising administering a pharmaceutical composition according to any of the embodiments 187-204 to the patient.
254. A process according to embodiment 252 or 253, wherein the patient is undergoing treatments that provide oxidative stress such as chemotherapy or radiation therapy.
255. A process according to any of embodiments 252-254, wherein the oxidative stress results in or contributes to mucositis in the patient.
256. A process according to embodiment 255, wherein the mucositis is oral mucositis.
257. A process for providing an antioxidant effect in a human or non-human animal patient comprising administering a pharmaceutical composition according to any of the embodiments 89-112 to the patient.
258. A process for providing an antioxidant effect in a human or non-human animal patient comprising administering a pharmaceutical composition according to any of the embodiments 187-204 to the patient.
259. A process according to embodiment 257 or 258, wherein the patient is undergoing chemotherapy or radiation therapy.
260. A process according to any of embodiments 257-259, wherein the patient has mucositis.
261. A process according to embodiment 260, wherein the mucositis is oral mucositis.
262. A process for providing one or more effects selected from the group consisting of slowing the onset of oxidative damage, reducing the severity of oxidative damage, and/or reducing the duration of oxidative damage in a human or non-human animal patient comprising administering a pharmaceutical composition according to any of the embodiments 89-112 to the patient.
263. A process for providing one or more effects selected from the group consisting of slowing the onset of oxidative damage, reducing the severity of oxidative damage, and/or reducing the duration of oxidative damage in a human or non-human animal patient comprising administering a pharmaceutical composition according to any of the embodiments 187-204 to the patient.
264. A process according to embodiment 262 or 263, wherein the patient is undergoing chemotherapy and/or radiation therapy.
265. A process according to any of embodiments 262-264, wherein the patient has mucositis.
266. A process according to embodiment 265, wherein the mucositis is oral mucositis.
267. A dry pharmaceutical admixture comprising (i) at least one active pharmaceutical ingredient that reduces the rate of cellular oxygen consumption (OCR-API), and (ii) a dry pharmaceutical composition, wherein the dry pharmaceutical composition comprises a composition according to any of embodiments 1-39 and 64-97 that substantially excludes water and any non-aqueous solvents.
268. A pharmaceutically acceptable container according to any of embodiments 119-127 comprising the dry pharmaceutical admixture of embodiment 267.
269. A liquid pharmaceutical composition prepared by admixing a dry pharmaceutical admixture according to embodiment 267 and a liquid that comprises water.
270. A liquid pharmaceutical composition prepared by admixing a dry pharmaceutical admixture according to embodiment 267 and a liquid that comprises a non-aqueous solvent.
271. A liquid pharmaceutical composition prepared by admixing a dry pharmaceutical admixture according to embodiment 267 and a liquid contained in a pharmaceutically acceptable container according to any of embodiments 119-127.
272. A liquid pharmaceutical composition according to embodiment 271, wherein the liquid comprises water.
273. A liquid pharmaceutical composition according to embodiment 271 or 272, wherein the liquid comprises a non-aqueous solvent.
274. A combination comprising (i) at least one active pharmaceutical ingredient that reduces the rate of cellular oxygen consumption (OCR-API), wherein such OCR-API is present in an aqueous or non-aqueous liquid form, and (ii) a dry pharmaceutical composition, wherein the dry pharmaceutical composition comprises a composition according to any of embodiments 1-39 and 64-97 that substantially excludes water and any non-aqueous solvents.
275. A combination according to embodiment 274, wherein the liquid OCR-API and dry pharmaceutical composition are present in a pharmaceutically acceptable container according to any of embodiments 121-127, and wherein the liquid OCR-API is present in the cavity and the dry pharmaceutical composition is present in the compartment separate from the cavity.
276. A liquid pharmaceutical composition prepared by admixing the liquid OCR-API and the dry pharmaceutical composition that are present in a pharmaceutically acceptable container according to embodiment 275.
277. A process according to any of embodiments 187-204, wherein the dry pharmaceutical composition further comprises at least one active pharmaceutical ingredient that reduces the rate of cellular oxygen consumption (OCR-API).
278. A process according to any of embodiments 205-230 and 243-266, wherein, in addition to administration of the oltipraz crystal composition, the patient is further administered at least one active pharmaceutical ingredient that reduces the rate of cellular oxygen consumption (OCR-API).
279. A process according to embodiment 278, wherein, for at least one administration, the oltipraz crystal composition is administered to the patient substantially together with the OCR-API.
280. A process according to embodiment 278, wherein, for at least one administration, the oltipraz crystal composition is administered at substantially different times than the OCR-API.
281. A process according to embodiment 278, wherein the OCR-API is first administered to the patient at least 12 hours prior to the first administration of an oltipraz crystal composition to the patient.
282. A process according to embodiment 278, wherein the OCR-API is first administered to the patient at least 24 hours prior to the first administration of an oltipraz crystal composition to the patient.
283. A process according to embodiment 278, wherein multiple doses of the OCR-APT are administered to the patient prior to the first administration of an oltipraz crystal composition to the patient.
284. A process according to embodiment 278, wherein the patient is undergoing radiation therapy and the administration of the OCR-API and oltipraz crystal composition is continued on a periodic basis until the completion of the radiation therapy.
285. A process for improving the action of a pharmaceutical composition comprising oltipraz crystals according to any of embodiments 89-118 for preventing, treating, ameliorating, lessening the severity and/or shortening the duration of mucositis for a human or non-human animal patient in need comprising co-administering to the patient at least one active pharmaceutical ingredient that reduces the rate of cellular oxygen consumption (OCR-API).
286. A pharmaceutical admixture comprising (i) at least one active pharmaceutical ingredient that reduces the rate of cellular oxygen consumption (OCR-API), and (ii) a dry pharmaceutical composition, wherein the dry pharmaceutical composition comprises oltipraz and substantially excludes water and any non-aqueous solvents.
287. A pharmaceutically acceptable container for providing an aqueous pharmaceutical composition, comprising a cavity of sufficient size to hold both a dry pharmaceutical composition and a quantity of liquid sufficient to permit mixing of the dry pharmaceutical composition to form a liquid composition, wherein the container comprises a dry pharmaceutical composition comprising oltipraz and at least one active pharmaceutical ingredient that reduces the rate of cellular oxygen consumption (OCR-API), and substantially excludes water and any non-aqueous solvents.
288. A pharmaceutically acceptable container according to embodiment 287, further comprising a releasable coupling for providing an opening in the container adapted to dispense a liquid composition from the container.
289. A pharmaceutically acceptable container according to embodiment 287, comprising a compartment separate from the cavity, said compartment comprising the dose of the dry pharmaceutical composition.
290. A pharmaceutically acceptable container according to embodiment 288, comprising a compartment separate from the cavity, said compartment comprising the dose of the dry pharmaceutical composition.
291. A pharmaceutically acceptable container according to embodiment 290, wherein the releasable coupling connects the portion of the container comprising the cavity to the portion of the container comprising the compartment.
292. A pharmaceutically acceptable container according to any of embodiments 287-291, further comprising a breakable seal between the compartment and the cavity.
293. A pharmaceutically acceptable container according to any of embodiments 287-292, further comprising a liquid comprising water.
294. A pharmaceutically acceptable container according to any of embodiments 287-293, further comprising a liquid comprising a non-aqueous solvent.
295. A pharmaceutically acceptable container according to embodiment 293 or 294, wherein the liquid also comprises at least one pharmaceutically acceptable taste-modifying additive.
296. A pharmaceutically acceptable container for providing an aqueous pharmaceutical composition, comprising a cavity of sufficient size to hold both a dry pharmaceutical composition and a quantity of liquid comprising an amount of water sufficient to permit mixing of the dry pharmaceutical composition to form a liquid composition, wherein container comprises (i) a dry pharmaceutical composition comprising oltipraz, wherein the dry pharmaceutical composition substantially excludes water and any non-aqueous solvents, and (ii) a quantity of liquid, wherein the quantity of liquid comprises at least one active pharmaceutical ingredient that reduces the rate of cellular oxygen consumption (OCR-API).
297. A pharmaceutically acceptable container according to embodiment 296, further comprising a releasable coupling for providing an opening in the container adapted to dispense a liquid composition from the container.
298. A pharmaceutically acceptable container according to embodiment 296, comprising a compartment separate from the cavity, wherein said compartment comprising the dose of the dry pharmaceutical composition and wherein the cavity comprises the liquid comprising the OCR-API.
299. A pharmaceutically acceptable container according to embodiment 297, comprising a compartment separate from the cavity, wherein said compartment comprising the dose of the dry pharmaceutical composition and wherein the cavity comprises the liquid comprising the OCR-API.
300. A pharmaceutically acceptable container according to embodiment 299, wherein the releasable coupling connects the portion of the container comprising the cavity to the portion of the container comprising the compartment.
301. A pharmaceutically acceptable container according to any of embodiments 296-300, further comprising a breakable seal between the compartment and the cavity.
302. A pharmaceutically acceptable container according to any of embodiments 296-301, wherein the liquid comprises water.
303. A pharmaceutically acceptable container according to any of embodiments 296-302, wherein the liquid comprises a non-aqueous solvent.
304. A pharmaceutically acceptable container according to embodiment 302 or 303, wherein the liquid also comprises at least one pharmaceutically acceptable taste-modifying additive.
305. A liquid pharmaceutical composition prepared by admixing the liquid comprising the dry pharmaceutical composition and the liquid that are present in a pharmaceutically acceptable container according to any of embodiments 287-304.
306. A pharmaceutical composition comprising (i) at least one active pharmaceutical ingredient that reduces the rate of cellular oxygen consumption (OCR-API) and (ii) oltipraz.
307. A liquid pharmaceutical composition comprising (i) at least one active pharmaceutical ingredient that reduces the rate of cellular oxygen consumption (OCR-APT), (ii) oltipraz, and (iii) water and/or a non-aqueous solvent.
308. A process for treating a human or non-human animal patient in need comprising administering to the patient a pharmaceutical composition according to any of embodiments 286, 306 or 307.
309. A process for treating a human or non-human animal patient in need comprising co-administering to the patient a (i) a pharmaceutical composition comprising oltipraz, and (ii) a pharmaceutical composition comprising at least one active pharmaceutical ingredient that reduces the rate of cellular oxygen consumption (OCR-API).
310. A process for preventing, treating, ameliorating, lessening the severity and/or shortening the duration of mucositis for a human or non-human animal patient in need comprising orally administering a pharmaceutical composition according to any of embodiments 286, 306 or 307 to the patient.
311. A process for preventing, treating, ameliorating, lessening the severity and/or shortening the duration of mucositis for a human or non-human animal patient in need comprising orally co-administering to the patient a (i) a pharmaceutical composition comprising oltipraz, and (ii) a pharmaceutical composition comprising at least one active pharmaceutical ingredient that reduces the rate of cellular oxygen consumption (OCR-APT).
312. A process according to embodiment 310 or 311, wherein the mucositis in oral mucositis.
313. A process according to embodiment 310 or 311, wherein the mucositis in mucositis of the alimentary canal.
314. A process for preventing, treating, ameliorating, lessening the severity and/or shortening the duration of mucositis for a human or non-human animal patient in need comprising topically administering a composition comprising oltipraz, and a composition comprising at least one active pharmaceutical ingredient that reduces the rate of cellular oxygen consumption (OCR-API).
315. A process for preventing, treating, ameliorating, lessening the severity and/or shortening the duration of mucositis for a human or non-human animal patient in need comprising rectally administering a composition comprising oltipraz, and a composition comprising at least one active pharmaceutical ingredient that reduces the rate of cellular oxygen consumption (OCR-API).
316. A process for preventing, treating, ameliorating, lessening the severity and/or shortening the duration of mucositis for a human or non-human animal patient in need comprising administering by inhalation a composition comprising oltipraz, and a composition comprising at least one active pharmaceutical ingredient that reduces the rate of cellular oxygen consumption (OCR-API).
317. A process according to any of embodiments 308-316, wherein the patient is undergoing radiation therapy.
318. A process according to any of embodiments 308-317, wherein, for at least one administration, the composition comprising oltipraz is administered to the patient substantially together with the composition comprising at least one OCR-APL 319. A process according to any of embodiments 308-317, wherein, for at least one administration, the composition comprising oltipraz is administered at substantially different times than the composition comprising the at least one OCR-API.
320. A process according to any of embodiments 308-317, wherein the composition comprising the at least one OCR-API is first administered to the patient at least 12 hours prior to the first administration to the patient of a composition comprising oltipraz.
321. A process according to any of embodiments 308-317, wherein the composition comprising the at least one OCR-API is first administered to the patient at least 24 hours prior to the first administration to the patient of a composition comprising oltipraz.
322. A process according to any of embodiments 308-317, wherein multiple doses of a composition comprising at least one OCR-API are administered to the patient prior to the first administration to the patient of a composition comprising oltipraz.
323. A process according to any of embodiments 308-317, wherein the patient is undergoing radiation therapy and the administration of the OCR-API and oltipraz compositions is continued on a periodic basis until the completion of the radiation therapy.
324. A process for improving the action of a pharmaceutical composition comprising oltipraz for preventing, treating, ameliorating, lessening the severity and/or shortening the duration of mucositis for a human or non-human animal patient in need comprising co-administering to the patient a composition comprising at least one active pharmaceutical ingredient that reduces the rate of cellular oxygen consumption (OCR-API).
325. A process according to any of embodiments 308-324, wherein the composition comprising oltipraz is also co-administered with at least one pharmaceutically acceptable agent selected from the group consisting of antioxidants, agents that enhance glutathione synthesis, glutathione, Medihoney. NF-kappaB inhibitors, anti-inflammatory agents, and compounds prevent damage from reactive Of (superoxide).
326. A process according to any of embodiments 308-324, wherein the composition comprising oltipraz is also co-administered with at least one pharmaceutically acceptable agent selected from the group consisting of N acetylcysteine, pantothenic acid (vitamin B5), glutathione. Medihoney, curcumin. Mesalamine, and superoxide dismutase.
327. A process for increasing the gene expression of glutathione peroxidase 4 (GPX4) and/or myeloperoxidase (MPO) in a human or non-human animal patient comprising administering to the patient a composition comprising oltipraz, and a composition comprising at least one active pharmaceutical ingredient that reduces the rate of cellular oxygen consumption (OCR-API).
328. A process for decreasing the gene expression of Peroxiredoxin 2 (PRDX2) in a human or non-human animal patient comprising administering to the patient a composition comprising oltipraz, and a composition comprising at least one active pharmaceutical ingredient that reduces the rate of cellular oxygen consumption (OCR-API).
329. A process for increasing the gene expression of glutathione peroxidase 4 (GPX4) and/or myeloperoxidase (MPO) and decreasing the gene expression of Peroxiredoxin 2 (PRDX2) in a human or non-human animal patient comprising administering to the patient a composition comprising oltipraz, and a composition comprising at least one active pharmaceutical ingredient that reduces the rate of cellular oxygen consumption (OCR-API).
330. A process for increasing the gene expression of glutathione peroxidase 4 (GPX4) and/or myeloperoxidase (MPO) in a human or non-human animal patient comprising administering to the patient a composition comprising oltipraz, and a composition comprising at least one active pharmaceutical ingredient that reduces the rate of cellular oxygen consumption (OCR-API).
331. A process for decreasing the gene expression of Peroxiredoxin 2 (PRDX2) in a human or non-human animal patient comprising administering to the patient a composition comprising oltipraz, and a composition comprising at least one active pharmaceutical ingredient that reduces the rate of cellular oxygen consumption (OCR-API).
332. A process for increasing the gene expression of glutathione peroxidase 4 (GPX4) and/or myeloperoxidase (MPO) and decreasing the gene expression of Peroxiredoxin 2 (PRDX2) in a human or non-human animal patient comprising administering a pharmaceutical composition according to any of the embodiments 187-204 to the patient.
333. A process according to any of embodiments 327-332, wherein the patient is undergoing chemotherapy and/or radiation therapy.
334. A process according to embodiment 333, wherein the patient has mucositis.
335. A process according to embodiment 334, wherein the mucositis is oral mucositis.
336. A process for decreasing intracellular reactive oxygen species (ROS) and/or decreasing oxidative stress in a human or non-human animal patient comprising administering to the patient a composition comprising oltipraz, and a composition comprising at least one active pharmaceutical ingredient that reduces the rate of cellular oxygen consumption (OCR-API).
337. A process for decreasing intracellular reactive oxygen species (ROS) and/or decreasing oxidative stress in a human or non-human animal patient comprising administering to the patient a composition comprising oltipraz, and a composition comprising at least one active pharmaceutical ingredient that reduces the rate of cellular oxygen consumption (OCR-API).
338. A process according to embodiment 336 or 337, wherein the patient is undergoing treatments that provide oxidative stress such as chemotherapy or radiation therapy.
339. A process according to any of embodiments 336-338, wherein the oxidative stress results in or contributes to mucositis in the patient.
340. A process according to embodiment 339, wherein the mucositis is oral mucositis.
341. A process for providing an antioxidant effect in a human or non-human animal patient comprising administering to the patient a composition comprising oltipraz, and a composition comprising at least one active pharmaceutical ingredient that reduces the rate of cellular oxygen consumption (OCR-API).
342. A process for providing an antioxidant effect in a human or non-human animal patient comprising administering to the patient a composition comprising oltipraz, and a composition comprising at least one active pharmaceutical ingredient that reduces the rate of cellular oxygen consumption (OCR-API).
343. A process according to embodiment 341 or 342, wherein the patient is undergoing chemotherapy or radiation therapy.
344. A process according to any of embodiments 341-343, wherein the patient has mucositis.
345. A process according to embodiment 344, wherein the mucositis is oral mucositis.
346. A process for providing one or more effects selected from the group consisting of slowing the onset of oxidative damage, reducing the severity of oxidative damage, and/or reducing the duration of oxidative damage in a human or non-human animal patient comprising administering to the patient a composition comprising oltipraz, and a composition comprising at least one active pharmaceutical ingredient that reduces the rate of cellular oxygen consumption (OCR-API).
347. A process according to embodiment 346, wherein the patient is undergoing chemotherapy and/or radiation therapy.
348. A process according to any of embodiments 346 or 347, wherein the patient has mucositis.
349. A process according to embodiment 348, wherein the mucositis is oral mucositis.
350. A process according to embodiment 309, wherein the patient is experiencing a condition or undergoing a procedure in which the patient may experience ischemia and/or reperfusion injury.
351. A process according to embodiment 350, wherein the co-administration prevents, treats, lessens the symptoms, and/or decreases the injury associated with ischemia and/or reperfusion injury.
352. A process according to embodiment 351 or 352, wherein the condition or procedure is selected from the group consisting of vascular repair, myocardial infarction, a procedure involving a clot removal, stroke, and organ transplantation.
353. A product or process according to any of embodiments 286-352, wherein the at least one OCR-API is selected from the group consisting of meclizine, nimorazole, metformin. phenformin, antimycin A, pyrvinium, berberine, niclosamide, acriflavinium, sorafenib, emetine, plicamycin, suloctidil, pentamidine, amsacrine, irinotecan, itraconazole, mitomycin, hydroxyprogesterone, cyclosporine, fenofibrate, atovaquone, an analogue of ubiquinone other than atovaquone, and mixtures thereof.
354. A product or process according to any of embodiments 286-353, wherein the at least one OCR-APT is atovaquone.
355. A product or process according to any of embodiments 286-354, wherein the at least one OCR-API is metformin and/or berberine.
356. A composition for storage, transport and/or reperfusion of organs prior to and/or during organ transplantation, wherein the composition comprises at least one OCR-API.
357. A composition according to embodiment 356, wherein the helps to prevent, treat, lessen the symptoms, and/or decrease the injury associated with reperfusion injury during or following organ transplantation.
358. A process for preventing, treating, lessening the symptoms, and/or decreasing the injury associated with reperfusion injury during or following transplantation comprising providing a composition comprising at least one OCR-API for storage, transport and/or reperfusion of the organ prior to and/or during transplantation.
359. A composition or process according to any of embodiments 356-358, wherein the at least one OCR-API is selected from the group consisting of meclizine, nimorazole, metformin, phenformin, antimycin A, pyrvinium, berberine, niclosamide, acriflavinium, sorafenib, emetine, plicamycin, suloctidil, pentamidine, amsacrine, irinotecan, itraconazole, mitomycin, hydroxyprogesterone, cyclosporine, fenofibrate, atovaquone, an analogue of ubiquinone other than atovaquone, and mixtures thereof.
360. A composition or process according to any of embodiments 356-359, wherein the at least one OCR-API is atovaquone.
361. A composition or process according to any of embodiments 356-360, wherein the at least one OCR-API is metformin and/or berberine.
362. A composition or process according to any of embodiments 356-361, wherein the composition further comprises an Nrt2 activator.
363. A composition or process according to embodiment 362, wherein the composition further comprises oltipraz.
364. A composition or process according to embodiment 362, wherein the Nrf2 activator comprises a compound selected from the group consisting of sulphoraphane, phenethyl isothiocyanate, oltipraz, curcumin, resveratrol, fumaric acid and its esters, synthetic oleanane triterpenoids, and combinations thereof.
365. A process for treating a human or non-human animal patient experiencing a condition or undergoing a procedure in which the patient may experience ischemia and/or reperfusion injury comprising administering to the patient a pharmaceutical composition comprising at least one Nrf2 activator.
366. A process according to embodiment 365, wherein the administration prevents, treats, lessens the symptoms, and/or decreases the injury associated with reperfusion injury.
367. A process according to embodiment 365 or 366, wherein the condition or procedure is selected from the group consisting of vascular repair, myocardial infarction, a procedure involving a clot removal, stroke, and organ transplantation.
368. A process for preventing, treating, lessening the symptoms, and/or decreasing the injury associated with reperfusion injury during or following transplantation comprising providing to the patient a composition comprising at least one Nrf2 activator prior to and/or during transplantation.
369. A process for storage, transport and/or reperfusion of an organ prior to and/or during organ transplantation, comprising exposing the organ to a composition comprising at least one Nrf2 activator.
370. A process according to any of embodiments 365-369, wherein the composition comprises at least one Nrf2 activator selected from the group consisting of sulphoraphane, phenethyl isothiocyanate, oltipraz, curcumin, resveratrol, fumaric acid and its esters, and synthetic oleanane triterpenoids.
371. A process according to embodiment 370, wherein the composition comprises oltipraz.
372. A process according to embodiment 371, wherein the composition comprises a dry or liquid composition comprising oltipraz formulated according to any of the embodiments set forth in Section A and C herein.
373. A process according to embodiment 371, wherein the composition comprises a dry pharmaceutical composition, wherein the dry pharmaceutical composition comprises a composition according to any of embodiments 1-39 and 64-97.
374. A process according to embodiment 373, wherein the dry pharmaceutical composition is provided in a pharmaceutically acceptable container according to any of embodiments 119-127.
375. A process according to embodiment 371, wherein the composition comprising oltipraz is a liquid pharmaceutical composition prepared by admixing a dry pharmaceutical admixture according to any of embodiments 1-39 and 64-97 and a liquid.
376. A process according to embodiment 375, wherein the liquid comprises water.
377. A process according to embodiment 375 or 376, wherein the liquid comprises a non-aqueous solvent.
378. A process according to claim 371, wherein the step of administering comprise the steps of
   providing a pharmaceutically acceptable container comprising a cavity, and
   adding to the container a dose of a dry pharmaceutical composition, wherein the dry pharmaceutical composition comprises a composition according to any of embodiments 1-39 and 64-99 that substantially excludes water,
   wherein the cavity is of sufficient size to hold both the dry pharmaceutical composition and an amount of a liquid sufficient to permit mixing of the dry pharmaceutical composition with a liquid to form a liquid pharmaceutical composition.
379. A process according to claim 371, wherein the step of administering comprise the steps of
   providing a pharmaceutically acceptable container comprising a cavity, and
   adding to the container a dose of a dry pharmaceutical composition, wherein the dry pharmaceutical composition comprises a dry composition prepared according to embodiment 176 or 177.
   wherein the cavity is of sufficient size to hold both the dry pharmaceutical composition and an amount of liquid sufficient to permit mixing of the dry pharmaceutical composition with a liquid to form a liquid pharmaceutical composition.
380. A process according to embodiment 378 or 379, wherein the container comprises a compartment separate from the cavity, and the dry pharmaceutical composition is added to the compartment.
381. A process according to any of embodiments 378-380, wherein the container comprises a releasable coupling for uncoupling a portion of the container to provide an opening for dispensing a liquid pharmaceutical composition from the container.
382. A process according to embodiment 381, wherein the container further comprises a releasable coupling for uncoupling a portion of the container to provide an opening for dispensing a liquid composition from the container, and wherein the releasable coupling connects the portion of the container comprising the cavity to the portion of the container comprising the compartment that contains the dose of a dry pharmaceutical composition.
383. A process according to any of embodiments 378-382, wherein the container further comprises a breakable seal between the compartment and the cavity, and wherein the dry pharmaceutical composition remains separate from the cavity when said seal is unbroken, and wherein the dry pharmaceutical composition can enter the cavity when the seal is broken.
384. A process according to any of embodiments 378-383, further comprises adding a liquid to the pharmaceutically acceptable container and mixing the liquid and dry pharmaceutical composition.
385. A process according to any of embodiments 378-383, wherein the step of administering comprises the steps of adding a liquid to the cavity of a pharmaceutically acceptable container according to any of embodiments 119-124, and mixing the dose of dry pharmaceutical composition according to any of embodiments 1-39 and 64-99 with the liquid.
386. A process according to embodiments 385, wherein the step of administering comprises the steps of adding a liquid to the cavity of a pharmaceutically acceptable container according to embodiment 122-124, causing the dry pharmaceutical composition in the compartment to enter the cavity, and mixing the dose of dry pharmaceutical composition with the liquid.
387. A process according to embodiments 385, wherein the step of administering comprises the steps of adding a liquid to the cavity of a pharmaceutically acceptable container according to embodiment 124, breaking the seal between the compartment and the cavity and causing the dry pharmaceutical composition in the compartment to enter the cavity, and mixing the dose of dry pharmaceutical composition with the liquid.
388. A process according to any of embodiments 384-387, wherein the liquid further comprises at least one pharmaceutically acceptable taste-modifying additive.
389. A process according to any of embodiments 384-387, wherein the step of mixing is carried out by shaking the container for ten minutes or less.
390. A process according to embodiment 389, wherein the step of mixing is carried out by shaking the container for five minutes or less.
391. A process according to embodiment 389, wherein the step of mixing is carried out by shaking the container for three minutes or less.
392. A process according to embodiment 389, wherein the step of mixing is carried out by shaking the container for two minutes or less.
393. A process according to embodiment 389, wherein the step of mixing is carried out by shaking the container for one minute or less.

### Definitions

For convenience, certain terms employed in the specification and appended claims are collected here. These definitions should be read in light of the entire disclosure and understood as by a person of skill in the art.

The articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined. i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B): in another embodiment, to B only (optionally including elements other than A); in yet another embodiment, to both A and B (optionally including other elements); etc.

The phrase “or,” as used herein in the specification and in the claims, should be understood to mean “either or both” of the elements so conjoined, i.e., elements that are conjunctively present in some cases and disjunctively present in other cases. Multiple elements listed with “or” should be construed in the same fashion, i.e., “one or more” of the elements so conjoined. Other elements may optionally be present other than the elements specifically identified by the “or” clause, whether related or unrelated to those elements specifically identified. Thus, as a non-limiting example, a reference to “A or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one.” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one. A, with no B present (and optionally including elements other than B); in another embodiment, to at least one, optionally including more than one, B, with no A present (and optionally including elements other than A); in yet another embodiment, to at least one, optionally including more than one. A, and at least one, optionally including more than one, B (and optionally including other elements); etc.

It should also be understood that, unless clearly indicated to the contrary, processes described herein and claimed below can include steps in addition to the steps recited, and the order of the steps or acts of the process is not necessarily limited to the order in which the steps or acts of the process are recited. In the context of this disclosure, the words “process” and “method” are synonymous.

In the claims, as well as in the specification, all transitional phrases such as “comprising,” “comprised of,” “including,” “carrying,” “having,” “containing,” “involving.” “holding,” “composed of” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures. Section 2111.03.

Those skilled in the art will recognize, or will be able to ascertain using no more than routine experimentation, many equivalents to the specific embodiments described herein. Such equivalents are intended to be encompassed by the following claims.

1. A composition which comprises (i) an active pharmaceutical ingredient that reduces the rate of cellular oxygen consumption (OCR-API) and (ii) 4-methyl-5-(pyrazin-2-yl)-3H-1,2-dithiole-3-thione (oltipraz) or a pharmaceutically acceptable salt thereof.

2. A composition according to claim 1, wherein the active pharmaceutical ingredient that reduces the rate of cellular oxygen consumption is selected from atovaquone, a ubiquinone analogue other than atovaquone, meclizine, nimorazole, metformin, phenformin, antimycin A, pyrvinium, berberine, niclosamide, acriflavinium, sorafenib, emetine, plicamycin, suloctidil, pentamidine, amsacrine, irinotecan, itraconazole, mitomycin, hydroxyprogesterone, cyclosporine, fenofibrate and pharmaceutically acceptable salts thereof.

3. A composition according to claim 1, wherein the OCR-API is atovaquone.

4. A composition according to claim 1, wherein the composition comprises (i) atovaquone and (ii) oltipraz.

5. A composition according to claim 1, wherein the composition comprises (ii) a quantity of crystals of oltipraz having an intensity averaged, mean hydrodynamic diameter (Z-average) (“MHD”) of from 30 to 2000 nm, as measured by dynamic light scattering at 25° C. on a suspension of the crystals in water at a concentration of 0.01 to 0.1 mg of crystals per mL of water.

6. A composition according to claim 5, wherein the MHD is from about 30 to about 1200 nm.

7. A composition according to claim 5, wherein a polydispersity index (PdI) of the crystals of oltipraz, defined as PdI=(σ/d)², wherein σ is the standard deviation and d is the mean hydrodynamic diameter (Z-average) of the crystals, is from about 0.10 to about 0.60.

8-9. (canceled)

10. A pharmaceutical composition comprising (a) a composition as defined in claim 1 and (b) one or more pharmaceutically acceptable carriers, diluents, or excipients.

11. (canceled)

12. A pharmaceutical composition according to claim 10, wherein the pharmaceutical composition is for oral, buccal, topical, rectal, or colonic administration or for administration by inhalation.

13. A method of treating a human or animal body comprising administering a composition according to claim 1.

14. A method of treating mucositis in a subject in need thereof, wherein the subject is undergoing radiation therapy or chemotherapy comprising administering a composition according to claim 2.

15-17. (canceled)

18. A product comprising (i) an active pharmaceutical ingredient that reduces the rate of cellular oxygen consumption and (ii) oltipraz as defined in claim 1 for simultaneous, separate or sequential use in the treatment of a reperfusion injury in a subject in need thereof, wherein the subject is experiencing a condition or undergoing a procedure selected from the group consisting of vascular repair, myocardial infarction, a procedure involving a clot removal, stroke, and organ transplantation.

19. (canceled)

20. A composition for storage, transport and/or reperfusion of an organ prior to and/or during transplantation, wherein the composition comprises at least one OCR-API and a Nrf2 activator selected from the group consisting of sulphoraphane, phenethyl isothiocyanate, oltipraz, curcumin, resveratrol, fumaric acid and its esters, synthetic oleanane triterpenoids, and combinations thereof.

21-22. (canceled)

23. A process for treating a human or non-human animal patient experiencing a condition or undergoing a procedure in which the patient may experience ischemia and/or reperfusion injury; or for preventing, treating, lessening the symptoms, and/or decreasing the injury associated with reperfusion injury during or following transplantation, the method comprising administering to the patient a pharmaceutical composition comprising at least one Nrf2 activator; wherein, when preventing, treating, lessening the symptoms, and/or decreasing the injury associated with reperfusion, the at least one Nrf2 activator may be administered prior to and/or during transplantation.

24. A process according to claim 23, wherein the administration prevents, treats, lessens the symptoms, and/or decreases the injury associated with reperfusion injury.

25. A process according to claim 23, wherein the condition or procedure is selected from the group consisting of vascular repair, myocardial infarction, a procedure involving a clot removal, stroke, and organ transplantation.

26-27. (canceled)

28. A process according to claim 23, wherein the composition comprises at least one Nrf2 activator selected from the group consisting of sulphoraphane, phenethyl isothiocyanate, oltipraz, curcumin, resveratrol, fumaric acid and its esters, and synthetic oleanane triterpenoids.

29. A process accordingly to claim 28, wherein the composition comprises oltipraz.

30. A process according to claim 29, wherein the composition comprises a quantity of crystals of oltipraz having an intensity averaged, mean hydrodynamic diameter (Z-average) (“MHD”) of from 30 to 2000 nm, as measured by dynamic light scattering at 25° C. on a suspension of the crystals in water at a concentration of 0.01 to 0.1 mg of crystals per mL of water, and wherein a polydispersity index (PdI) of the crystals of oltipraz, defined as PdI=(σ/d)², wherein σ is the standard deviation and d is the mean hydrodynamic diameter (Z-average) of the crystals, is from about 0.10 to about 0.60.

31-33. (canceled)
